(19) United States
(12) Patent Application Publication
(10) Pub. No.: US 2026/0294330 A1
(43) Pub. Date: Oct. 1, 2026

(54) Predictive Neurological Monitoring and Clinical Decision Support System

(71) Applicant: PAHK NEUROLOGY INC., La Mirada, CA (US)

(72) Inventors: Eugene Pahk, La Mirada, CA (US); Kristians Veters, Jurmala (LV); Artjoms Simutins, Jurmala (LV)

(21) Appl. No.: 19/572,624

(22) Filed: Mar. 19, 2026

Related U.S. Application Data
(60) Provisional application No. 63/781,370, filed on Apr. 1, 2025.

(51) Int. Cl. A61B 5/00; A61B 5/11; A61B 5/16; G16H 50/20

(52) U.S. Cl. CPC A61B 5/4088; A61B 5/0022; A61B 5/1101; A61B 5/162; A61B 5/6898; A61B 5/7267; A61B 5/7275; A61B 5/7435; A61B 5/746; A61B 5/7475; G16H 50/20; A61B 2562/0219

(57) ABSTRACT

A system and method for predictive neurological assessment and clinical decision support utilizing mobile device sensor fusion. A mobile computing device presents digitized cognitive testing modules and simultaneously captures both foreground interaction data and background biometric sensor data, such as micro-oscillations, haptic pressure, and millisecond-level interaction latencies. The captured data is transmitted to a remote server where a trained machine learning model processes the background biometric data to detect sub-perceptual biometric anomalies. By comparing these anomalies against historical baseline patterns and supervised clinical diagnosis flags, the system generates a predictive trajectory vector to forecast the onset of a neurological condition prior to a macroscopic decline in foreground test scores. The system outputs early-warning alerts, temporal prognoses, and treatment efficacy tracking to a secure clinician portal to facilitate predictive medical interventions.

### CROSS REFERENCE TO RELATED APPLICATIONS

This application claims the benefits of Provisional Patent Application Ser. No. 63/781,370, filed on 2025 Apr. 1, all of which is expressly incorporated herein by reference.

### STATEMENT RE: FEDERALLY SPONSORED RESEARCH/DEVELOPMENT

Not Applicable.

### BACKGROUND

The various aspects and embodiments described herein relate to a system and method for predictive neurological assessment and clinical decision support utilizing mobile device sensor fusion. Neurological and cognitive assessments are vital for diagnosing and managing neurodegenerative diseases, motor function disorders, and other cognitive impairments. Traditionally, these assessments are administered periodically in clinical settings and rely on macroscopic, point-in-time evaluations of a patient's performance. However, conventional testing methodologies often fail to detect the earliest stages of neurological decline, as they typically require a substantial and observable degradation in a patient's test scores before a formal diagnosis can be made or a treatment plan initiated. Furthermore, the infrequent nature of these clinical evaluations limits the ability of medical professionals to continuously monitor a patient's daily cognitive and motor function, making it difficult to proactively track disease progression or rapidly evaluate the efficacy of prescribed treatments.

Accordingly, there remains a significant need in the art for an improved system capable of continuously and objectively monitoring subtle, sub-clinical changes in a patient's neurological health to facilitate earlier detection, predictive forecasting, and more responsive clinical interventions.

### BRIEF SUMMARY

In various exemplary embodiments, the present disclosure provides a system and method for predictive neurological assessment and clinical decision support utilizing mobile device sensor fusion. The present system advantageously overcomes the limitations of traditional, reactive cognitive testing by concurrently capturing two distinct layers of data while a user interacts with digitized cognitive modules or gamified applications on a mobile device. A first layer captures foreground interaction data to evaluate macroscopic task completion, while a second layer simultaneously captures high-fidelity background biometric sensor data—such as micro-oscillations, haptic pressure, continuous spatial coordinates, and millisecond-level inter-tap latencies—utilizing the mobile device's integrated hardware sensors. This dual-layer data is transmitted to a remote cloud-based server where a trained machine learning architecture processes the background biometric data to detect sub-perceptual anomalies. By performing retrospective analyses based on supervised clinical diagnosis flags and treatment initiation flags across a patient population, the machine learning model continuously learns and refines early-warning biometric signatures. Consequently, the system generates predictive trajectory vectors that dynamically forecast the future onset of neurological conditions, provide temporal prognoses, and evaluate the efficacy of prescribed treatments well before any macroscopic decline or improvement is evident in the foreground test scores. These predictive insights and corresponding treatment plan recommendations are subsequently outputted to a secure clinician portal, thereby transforming neurological monitoring into a proactive, continuous, and highly granular clinical decision support ecosystem.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a schematic diagram illustrating the overall neurological monitoring system architecture including mobile devices, a network, a cloud server, and a clinician portal.

FIG. 2A is a graphical user interface screen showing a home dashboard of the mobile application displaying daily brain training metrics and a button for initiating a training session.

FIG. 2B is a graphical user interface screen showing a module progress interface illustrating previously completed cognitive exercises.

FIG. 2C is a graphical user interface screen displaying a searchable library of clinical cognitive testing modules available within the application.

FIG. 2D is a graphical user interface screen displaying a selectable list of gamified cognitive exercises available within the application.

FIG. 2E is a graphical user interface screen showing a leaderboard and a list of historical cognitive test results for the user.

FIG. 2F is a graphical user interface screen displaying wellness metrics including daily step counts and a seven-day activity trend graph.

FIG. 2G is a graphical user interface screen displaying wellness metrics including floors climbed and a corresponding trend graph.

FIG. 2H is a graphical user interface screen showing a cognitive performance dashboard including an aggregate score and longitudinal trends across multiple cognitive domains.

FIG. 3A is a clinician portal interface displaying a patient management dashboard including a list of monitored patients and associated metadata.

FIG. 3B is a graphical visualization illustrating longitudinal neurological performance metrics plotted over time for a patient.

FIG. 3C is a graphical visualization illustrating AI-generated predictive trajectory vectors forecasting future neurological trends.

FIG. 3D is a flowchart illustrating a closed-loop clinical workflow including test administration, monitoring, diagnosis, and treatment evaluation.

FIG. 4A is a graphical user interface screen illustrating an alternating sequence cognitive test in which a user selects alphanumeric targets in alternating order.

FIG. 4B is a graphical user interface screen displaying a results page showing the user's score following completion of the alternating sequence test.

FIG. 5A is a graphical user interface screen presenting instructions for a drawing challenge cognitive test.

FIG. 5B is a graphical user interface screen illustrating a user-generated drawing created during the drawing challenge test.

FIG. 5C is a graphical user interface screen displaying evaluation feedback and a score for the completed drawing challenge.

FIG. 5D is a graphical user interface screen presenting a second drawing prompt involving a musical instrument.

FIG. 5E is a graphical user interface screen displaying feedback and scoring for the musical instrument drawing.

FIG. 5F is a graphical user interface screen displaying a completion summary for the drawing challenge session including an exercise score and total working time.

FIG. 6A is a graphical user interface screen presenting an introductory interface for a similarities cognitive test in which a user is instructed to identify conceptual relationships between two presented words.

FIG. 6B is a graphical user interface screen illustrating a similarities test prompt displaying a pair of words and a response input field for entering the conceptual relationship.

FIG. 6C is a graphical user interface screen displaying feedback and a score following completion of a similarities test question.

FIG. 6D is a graphical user interface screen illustrating a subsequent similarities exercise presenting an additional pair of conceptually related words.

FIG. 6E is a graphical user interface screen displaying feedback and scoring information for the subsequent similarities exercise.

FIG. 7A is a graphical user interface screen presenting an introductory interface for a fluency and language testing module in which a user is instructed to generate words belonging to a specified category.

FIG. 7B is a graphical user interface screen illustrating user responses being entered into a text field during a timed verbal fluency exercise.

FIG. 7C is a graphical user interface screen displaying a results page showing a performance score and feedback following completion of a fluency exercise.

FIG. 7D is a graphical user interface screen presenting a second fluency exercise requiring the user to generate words beginning with a specified letter.

FIG. 7E is a graphical user interface screen displaying feedback and scoring information for the second fluency exercise.

FIG. 8A is a graphical user interface screen presenting instructions for a number sequence sorting cognitive test in which a user listens to a spoken sequence of numbers and later enters the numbers in ascending order.

FIG. 8B is a graphical user interface screen illustrating playback of a spoken number sequence through the mobile device speaker.

FIG. 8C is a graphical user interface screen displaying an input interface including a numeric keypad for entering the reconstructed number sequence.

FIG. 8D is a graphical user interface screen illustrating a partially reconstructed sequence entered by the user.

FIG. 8E is a graphical user interface screen displaying a submission interface allowing the user to review and submit the reconstructed number sequence.

FIG. 8F is a graphical user interface screen displaying a results page including a performance score and explanatory feedback for the sequence sorting exercise.

FIG. 8G is a graphical user interface screen displaying a completion summary for the number sequence sorting session including the exercise score and total working time.

FIG. 9A is a graphical user interface screen presenting an object naming cognitive test prompt displaying an image of an object and a response field for entering the object name.

FIG. 9B is a graphical user interface screen illustrating user entry of an object name using an on-screen keyboard.

FIG. 9C is a graphical user interface screen displaying a subsequent object naming exercise presenting a new object image.

FIG. 9D is a graphical user interface screen illustrating an additional object naming prompt requiring the user to identify another displayed object.

FIG. 9E is a graphical user interface screen displaying feedback and a performance score following completion of the object naming exercise.

FIG. 9F is a graphical user interface screen displaying a completion summary for the object naming session.

FIG. 10A is a graphical user interface screen presenting instructions for a path-following cognitive test in which a user traces a predefined path on the touchscreen.

FIG. 10B is a graphical user interface screen illustrating a user tracing the path while a moving indicator tracks finger position.

FIG. 10C is a graphical user interface screen displaying progress along the path and a percentage completion indicator during the tracing activity.

FIG. 10D is a graphical user interface screen displaying feedback and a score following completion of the path-following task.

FIG. 10E is a graphical user interface screen displaying a completion summary for the path-following exercise session.

FIG. 11A is a graphical user interface screen presenting instructions for a tremor video test requiring the user to extend their arm while holding the mobile device.

FIG. 11B is a graphical user interface screen illustrating a recording interface capturing motion and video data during the tremor measurement interval.

FIG. 11C is a graphical user interface screen displaying a completion message following the tremor recording session.

FIG. 11D is a graphical user interface screen presenting instructions for a postural stability test in which the user stands still while holding the mobile device.

FIG. 11E is a graphical user interface screen illustrating completion of the postural stability test.

FIG. 12A is a graphical user interface screen presenting a quick math cognitive test prompt displaying a mathematical expression and numeric input field.

FIG. 12B is a graphical user interface screen displaying a results page including a score and feedback following submission of a calculated answer.

FIG. 12C is a graphical user interface screen illustrating a subsequent arithmetic problem presented within the quick math exercise sequence.

FIG. 13A is a graphical user interface screen presenting a reaction time and color recognition test instructing the user to select a target object of a specified color.

FIG. 13B is a graphical user interface screen displaying feedback and a score following successful selection of the correct colored target.

FIG. 13C is a graphical user interface screen illustrating a subsequent reaction-time exercise presenting a new color instruction and set of visual targets.

FIG. 14A is a graphical user interface screen presenting instructions for a sequence memory cognitive test in which numbered indicators appear within a grid for memorization.

FIG. 14B is a graphical user interface screen displaying feedback and a score following completion of a sequence memory exercise.

FIG. 14C is a graphical user interface screen illustrating a subsequent memorization phase showing numbered indicators within different grid positions.

FIG. 14D is a graphical user interface screen illustrating user selection of grid cells to reproduce the memorized sequence.

FIG. 14E is a graphical user interface screen displaying feedback and scoring information for a later sequence memory exercise.

FIG. 14F is a graphical user interface screen displaying a completion summary for the sequence memory exercise session.

FIG. 15A is a graphical user interface screen presenting instructions for a tremor assessment module requiring the user to hold the mobile device and extend the arm during recording.

FIG. 15B is a graphical user interface screen displaying a status message indicating completion of the tremor recording interval.

FIG. 15C is a graphical user interface screen displaying a completion summary for the tremor assessment session.

FIG. 16A is a graphical user interface screen presenting instructions for a visual reproduction cognitive test in which the user memorizes a geometric design.

FIG. 16B is a graphical user interface screen illustrating a drawing workspace where the user reproduces the memorized design.

FIG. 16C is a graphical user interface screen displaying feedback and a score evaluating the accuracy of the reproduced design.

FIG. 16D is a graphical user interface screen presenting a second geometric design for memorization.

FIG. 16E is a graphical user interface screen displaying a drawing workspace for reproducing the second memorized design.

FIG. 16F is a graphical user interface screen illustrating a user-generated drawing created during the visual reproduction exercise.

FIG. 16G is a graphical user interface screen displaying a completion summary for the visual reproduction session.

FIG. 17A is a graphical user interface screen presenting a voice recording and speech assessment module in which the user records themselves speaking a displayed phrase.

FIG. 17B is a graphical user interface screen displaying feedback and a score following analysis of the recorded speech.

FIG. 18A is a graphical user interface screen presenting a word scramble cognitive test in which a user rearranges scrambled letters to form a valid word.

FIG. 18B is a graphical user interface screen illustrating entry of a reconstructed word in response to a scrambled letter sequence.

FIG. 18C is a graphical user interface screen displaying feedback and a score following completion of the word scramble exercise.

### DETAILED DESCRIPTION

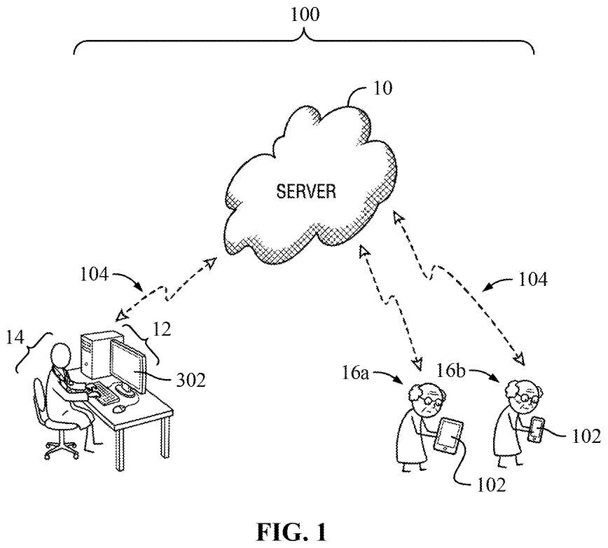

Referring now to FIG. 1, a schematic overview of the neurological assessment and predictive treatment system 100 is illustrated. The system 100 comprises one or more mobile computing devices 102 utilized by a plurality of patients, illustrated as users 16a and 16b. These mobile devices 102, which may encompass smartphones, tablet computers, or other portable computing apparatuses, comprise integrated hardware sensors such as capacitive touch digitizers, multi-axis accelerometers, gyroscopes, and high-resolution operating system event loggers configured to capture both foreground task inputs and background task inputs and biometric metadata. As the users 16a and 16b interact with the digitized cognitive testing modules on their mobile devices 102, the mobile devices 102 securely transmit via a network 104 the captured dual-layer sensor data over a wireless communication network to a remote cloud-based server 10. The server 10 houses the advanced analytics and machine learning (ML) architecture responsible for processing the raw timestamps, spatial coordinates, and motion data to calculate foreground cognitive scores and detect sub-perceptual biometric anomalies. After the AI-driven analysis is performed on the data, the processed neurological health metrics, predictive forecasting vectors, and identified biomarker patterns are transmitted from the server 10 to a secure clinician portal 12. A medical professional or clinician 14 interfaces with the clinician portal 12 via a desktop computer or specialized terminal to review the processed neurological metrics, underlying data values, and corresponding graphical visualizations illustrating trends over time.. Through the clinician portal 12, the clinician 14 can remotely configure specific testing modules for the users 16a and 16b, input supervised clinical diagnosis flags (discussed herein) to train the AI system, actively track the efficacy of previously prescribed therapies and flag improvements to train the AI system, and provide potential specific treatment protocols based on the actionable processed neurological metrics, underlying data values, and predictive analyses generated by the server 10.

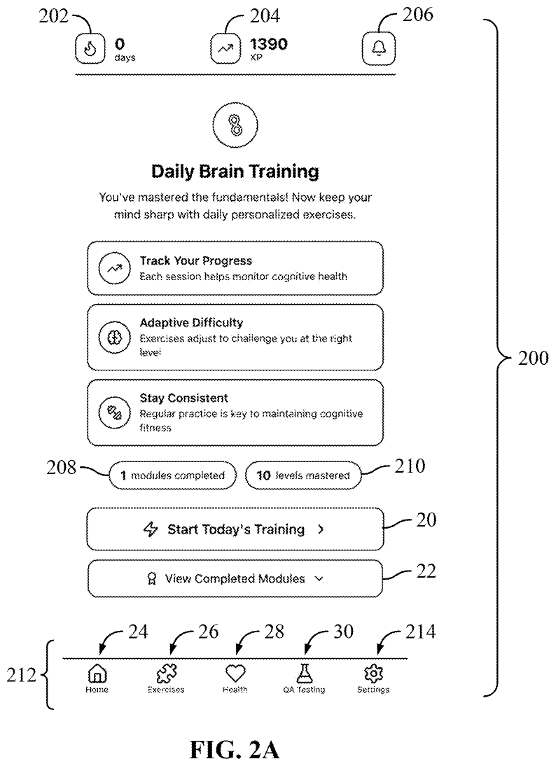

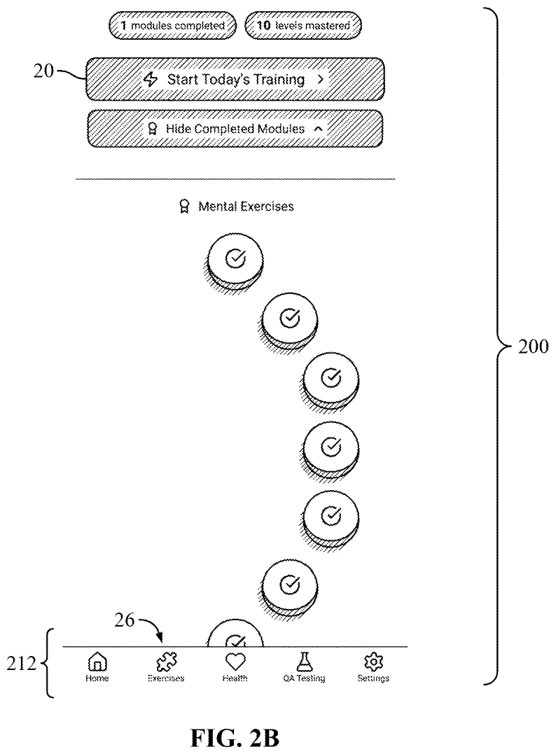

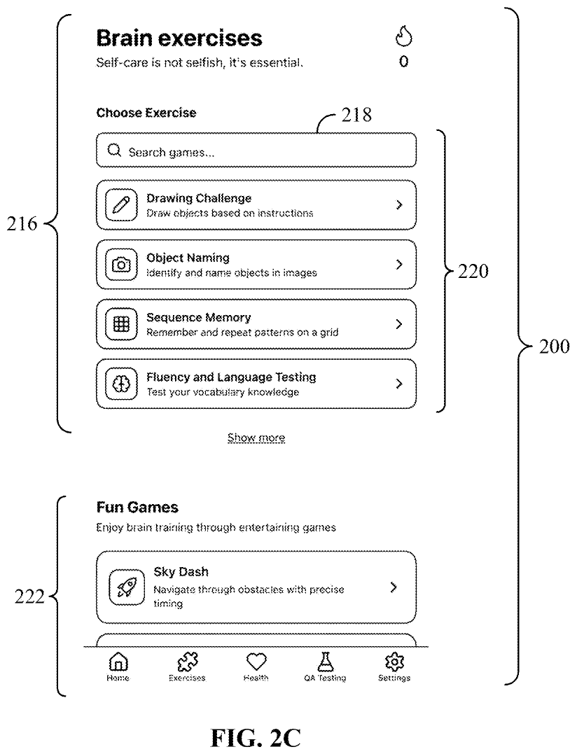

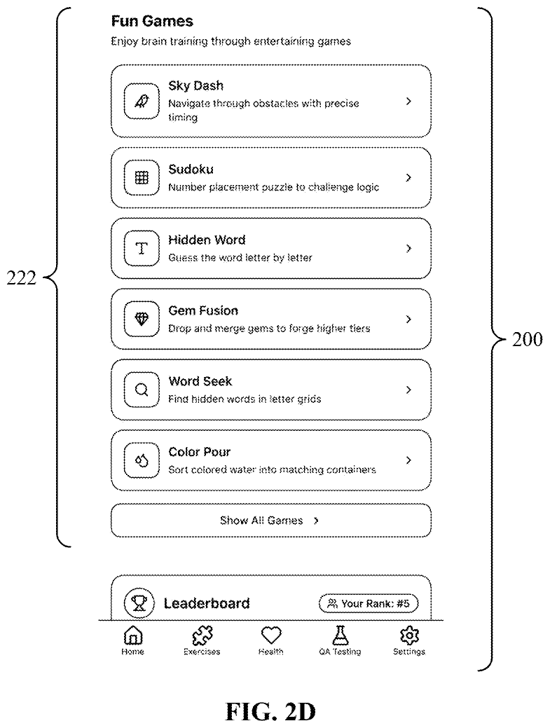

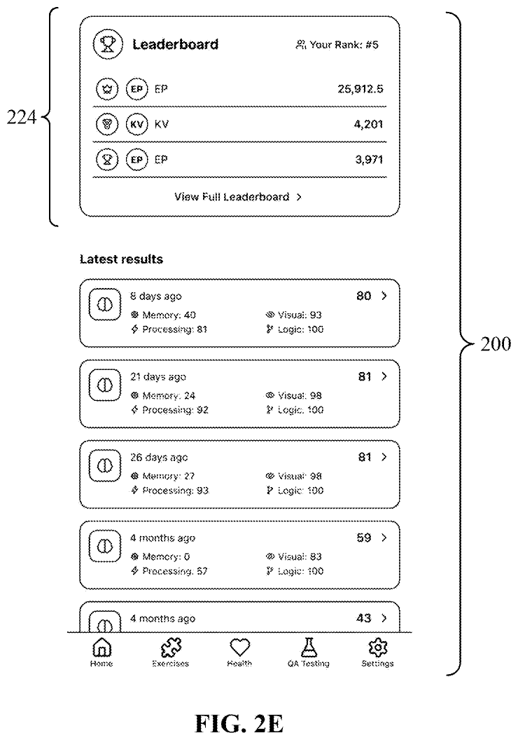

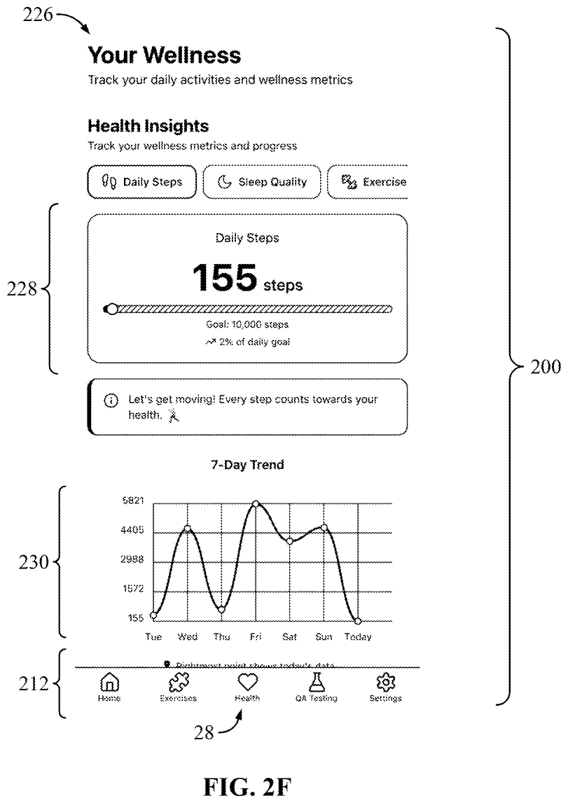

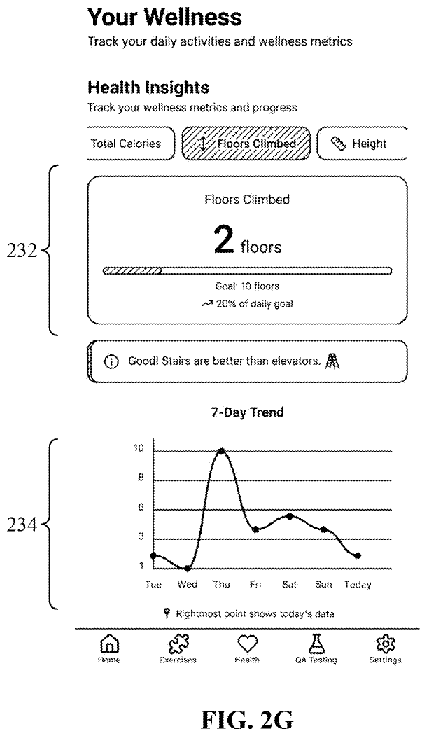

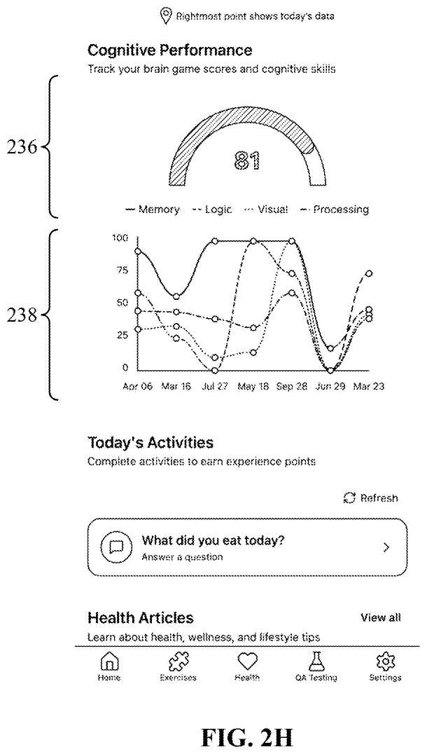

Referring now to FIGS. 2A through 2H, various graphical user interface (GUI) screens 200 of the mobile application executing on the patient's mobile device 102 are illustrated. FIG. 2A depicts a primary home screen presented to the user 16a, 16b upon launching the application. At the top of the interface, the system 100 displays real-time user engagement metrics, including an activity streak indicator 202 (e.g., a flame icon indicating consecutive days of use), an accumulated experience point (XP) tracker 204 to incentivize continued use, and a notification icon 206 for system or clinician alerts. The central body of the GUI screen 200 provides motivational prompts and dynamic text explaining the benefits of the application, alongside aggregate statistics such as the number of modules completed 208 and levels mastered 210. Positioned prominently on the GUI screen 200 is a primary execution button 20, labeled “Start Today's Training,” which the user 16a, 16b selects to initiate a daily, personalized list of cognitive assessments. Below this is a secondary toggle button 22 configured to reveal or hide previously completed modules 208. The bottom of the GUI screen 200 has a persistent navigation menu 212 comprising a plurality of selectable tabs, including a Home tab 24, an Exercises tab 26, a Health tab 28, a QA Testing tab 30, and a Settings tab 214.

As illustrated in FIG. 2B, the user 16a, 16b interacts with the navigation menu 212—specifically selecting the Exercises tab 26—to reach a module tracking interface. This GUI screen 200 displays a visual pathway of previously completed mental exercises, represented by a series of checkmarks, allowing the user 16a, 16b to visually conceptualize their longitudinal progress (i.e., progress over time (days, weeks, months, years)). To begin a new session, the patient 16a, 16b clicks on the “Start Today's Training” execution button 20 to initiate the specific sequence of tests scheduled for that patient 16a, 16b by the system 100 or remotely prescribed by the clinician 14.

Upon interacting with the application to view the full library of available modules, the patient 16a, 16b is directed to a comprehensive selection interface, illustrated collectively across FIGS. 2C, 2D, and 2E. These figures represent a single, continuous GUI screen 200 that the user 16a, 16b can scroll up and down. At the top of this scrollable interface (FIG. 2C), a “Brain exercises” section 216 provides a search bar 218 and a list of specific clinical cognitive modules 220, such as a “Drawing Challenge,” “Object Naming,” “Sequence Memory,” and “Fluency and Language Testing.” Other modules are also contemplated. Scrolling further down the GUI screen 200 (FIG. 2D) reveals a “Fun Games” subsection presenting gamified cognitive tests 222, such as “Sky Dash,” “Sudoku,” and “Word Seek.” Other tests are also contemplated. The inclusion of these gamified cognitive tests 222 ensures high patient compliance and retention because the application is fun to use, enabling the system 100 to continuously gather background sensor data while the user 16a, 16b engages in entertaining tasks, and not just clinical cognitive modules. Continuing to scroll to the bottom of the interface (FIG. 2E) reveals a “Leaderboard” section 224. This injects a degree of friendly competition into the application, motivating users 16a, 16b to return daily and thereby helping to aggregate the continuous longitudinal data required for the predictive AI models of the server 10. Below the Leaderboard section 224, a “Latest results” log displays the patient's historical test scores broken down into specific neurological domains, including Memory, Visual, Processing, and Logic.

Referring to FIGS. 2F and 2G, the user interface includes a “Your Wellness” health tracking section 226, accessed via the navigation menu 212 by clicking on the health icon 28. These GUI screens 200 display integrated physical health metrics that serve as contextual data for the AI system on the server 10. For example, FIG. 2F displays a daily step count 228 alongside a 7-day historical trend graph 230, while FIG. 2G displays floors climbed 232 and its respective 7-day trend graph 234. The system 100 may also track sleep quality and total exercise, as shown in FIG. 2G. By collecting this wellness data, the remote cloud server 10 and AI architecture can include this data when it does its retrospective analysis (discussed herein) and filter out confounding variables; for instance, determining that a temporary dip in a user's cognitive processing score is correlated with a severe lack of sleep rather than an actual neurological decline.

FIG. 2H illustrates a Cognitive Performance dashboard providing a highly detailed longitudinal visualization for the patient 16a, 16b. A primary aggregate score dial 236 (e.g., “81”) provides a high-level summary of the user's current cognitive state. Below the aggregate score dial 236, the GUI renders a complex time-series graph 238 that independently plots the trajectory of distinct cognitive domains—Memory, Logic, Visual, and Processing—over a span of months. Crucially, these four categories are patient-facing, user-friendly abstractions designed specifically for gamification and engagement. A clinician 14 does not formulate a medical diagnosis based on an abstracted “Logic Score.” Instead, these patient-facing information and visualizations serve to sustain the daily fun and user interactions desired for the user to continue to open the application on the smartphone so that the underlying hardware sensors of the mobile device 102 can continuously capture the necessary granular data and provide a clinician with decision support data and information which is accessible the clinician via the clinician portal 12.

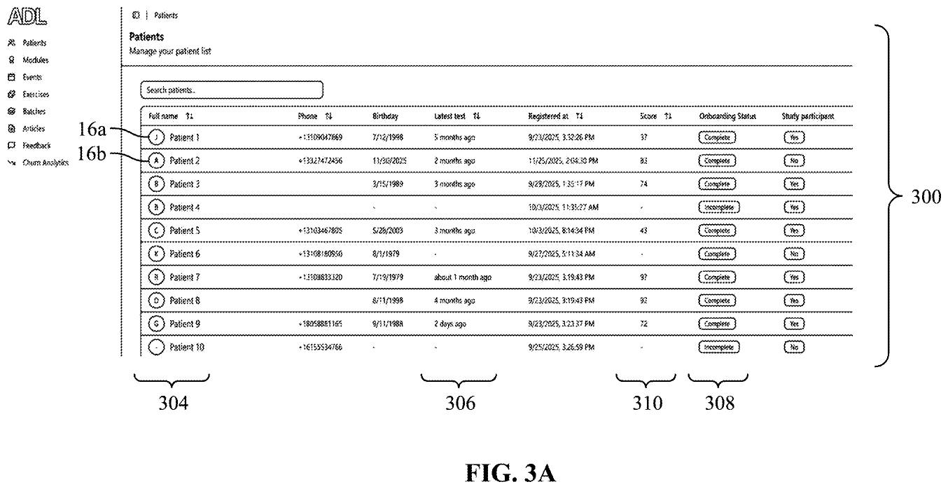

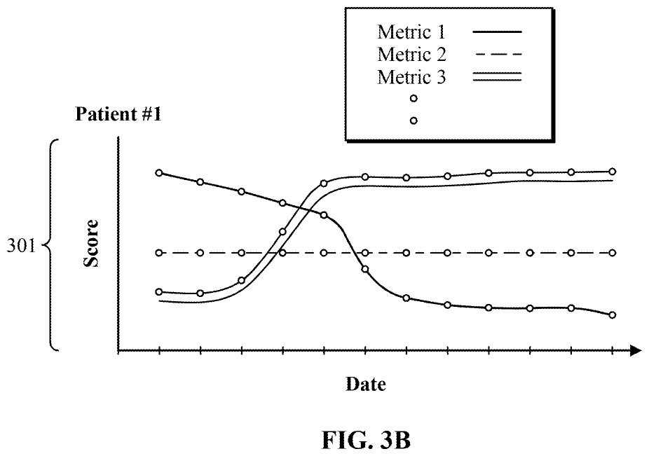

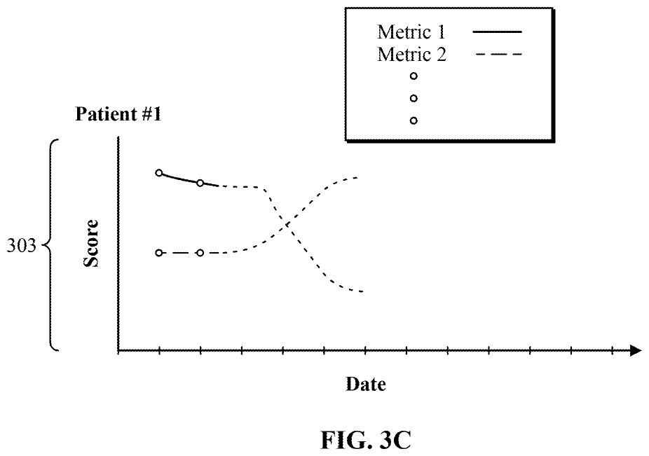

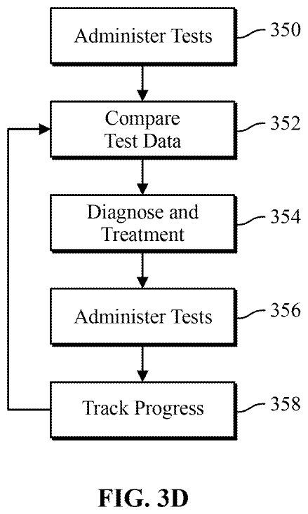

Referring now to FIGS. 3A through 3D, various views of a secure clinician portal GUI 300 and a corresponding clinical method flow are illustrated. The clinician portal GUI 300 is generated by the remote cloud-based server 10 and rendered on a display device 302 (see FIG. 1) of a medical professional, providing an actionable interface to review the highly complex, algorithmic back-end data that is purposefully hidden from the patient-facing application.

FIG. 3A illustrates a primary patient management dashboard of the clinician portal GUI 300. The clinician portal GUI 300 renders a roster 304 of monitored patients 16a, 16b, displaying critical metadata including a latest test date 306, an onboarding status 308, and an aggregate foreground task score 310. This dashboard acts as the primary triage interface, allowing the clinician 14 to quickly identify patients 16a, 16b requiring deeper review. Upon selecting a specific patient 16a from the roster 304, the clinician portal 12 queries the remote server 10 and transitions to the patient-specific graphical analytics interfaces illustrated in FIGS. 3B and 3C.

FIG. 3B illustrates a longitudinal data visualization mapping 301 of the patient's historical metrics over time. The X-axis represents chronological time, while the Y-axis represents calculated metric scores. Unlike the gamified patient application, the clinician portal 12 evaluates the underlying raw granular data (Layer 1) and background biometric data (Layer 2). This includes evaluating the exact millisecond reaction times, absolute target recall counts, sequence accuracy, and specific physical tremor amplitudes captured by the mobile device's 102 accelerometer and touch screen. Furthermore, a single poor test score does not trigger a treatment plan. Instead, the server 10 calculates complex derived longitudinal metrics based on the data trend over time. The lines plotted in FIG. 3B represent these derived metrics, which show a rate of decline measuring the velocity or acceleration of the patient's deterioration; a change-from-baseline magnitude calculating the delta between the patient's current state and their personalized historical normal; multi-domain impairment patterns detecting concurrent failures across distinct neurological areas, such as a simultaneous drop in memory and an increase in tremor amplitude; and safety-related motor changes, such as severe postural sway detected by the accelerometer indicating an acute fall risk. By way of example and not limitation, the distinct neurological areas may be include episodic memory, working memory, executive function, language production and lexical retrieval, semantic reasoning and abstraction, attention and sustained concentration, processing speed, visuospatial processing, visuoconstructional ability, fine motor control, visuomotor coordination, motor stability and tremor, balance and postural stability, speech motor control, and temporal orientation.

FIG. 3C illustrates a predictive forecasting visualization 303 generated by the system's 100 Artificial Intelligence (AI) and Machine Learning (ML) architecture. In this graphical representation, the clinician portal GUI 300 renders dashed lines extending forward along the chronological X-axis representing potential predictive trajectory vectors of the various metrics being tracked. The various metrics may be the same distinct neurological areas discussed above in relation to FIG. 3B. Rather than merely plotting past data, the remote server 10 processes the patient's historical test data—which may comprise the high-fidelity background sensor data alone, the foreground task data alone, or a synergistic combination of both the foreground and background sensor data—through a supervised neural network to forecast the patient's future neurological trajectory. The dashed lines clearly indicate to the clinician 14 whether a specific derived longitudinal metric is predicted to improve or further degrade in the coming months. Consequently, the prediction generated by the AI system is not only whether the patient 16a, 16b will develop a particular disease, but also provides a specific temporal prognosis indicating exactly how much longer the person has before severe degradation or disease onset occurs, depending on the past historical AI analysis lookback across the system 100. By identifying these predictive patterns, the system 100 empowers the clinician 14 to preemptively identify an impending condition. Furthermore, the predictive dashed lines may be utilized to track treatment efficacy. Once a treatment protocol is prescribed, the system's 100 AI continuously analyzes the incoming foreground and background sensor data, or a combination thereof, as the user continues to engage with the software application on the mobile device to predict whether the prescribed treatment is working, providing the clinician 14 with early confirmation of treatment efficacy based on physiological sensor stabilization long before traditional tests register an improvement.

FIG. 3D illustrates a schematic flowchart of the closed-loop clinical method executed by the system 100. The method operates as a sophisticated clinical decision support workflow. The protocol begins with a baseline establishment phase over multiple sessions, wherein the system 100 administers tests at step 350 via the mobile device 102 to collect raw data and establish a personalized normal for both cognitive and motor tasks without triggering diagnostic alerts. Diagnostic alerts are turned off for the first 3 months of use by the user. The system 100 then enters a continuous session generation phase, wherein the smartphone 102 continuously transmits raw data and the server 10 continuously calculates the derived longitudinal features whenever the smartphone and the application are being used by the user. During the subsequent algorithmic monitoring phase, the server 10 continuously compares at step 352 the newly generated session data to the established baseline, specifically scanning for sustained decline, rate of decline, increased day-to-day variability, cross-domain impairments, and safety risks. If one of these algorithmic triggers is met, the system 100 outputs an alert to the clinician portal 12. Crucially, the system 100 functions strictly as a longitudinal monitoring and decision support tool rather than an autonomous diagnostic device. Upon receiving the alert and reviewing the derived metrics on the clinician portal 12, the physician executes a treatment synthesis phase, interpreting the AI-generated findings in context with the patient's medical history, current medications, laboratory results, and neuroimaging before synthesizing a possible diagnosis and treatment at step 354. The system 100 then loops back to re-administering tests at step 356 to track the progress of the prescribed treatment at step 358. Alerts are turned off at this point to allow the treatment plan to show up in the test results.

### Digitized Cognitive Testing Modules

The following describes a series of digitized cognitive testing modules that may be implemented within a software application executing on the mobile device 102. The software application operating on the mobile device 102 functions as part of the system 100 to capture user interaction data and associated sensor data from the users 16a, 16b, which is subsequently processed by the server 10 to provide neurological monitoring and clinical decision support to the clinician 14 as described herein.

In various embodiments, each cognitive testing module executed on the mobile device 102 generates two categories of data streams. A first category comprises foreground interaction data that is necessary to administer and score the test itself, such as touch selections, drawing strokes, or object selections. A second category comprises background biometric sensor data captured simultaneously by sensors of the mobile device that are not required to administer the test but that provide additional physiological and behavioral information about the user. These two data layers may be captured concurrently and transmitted together to the server 10 for analysis.

### Alternating Sequence Test

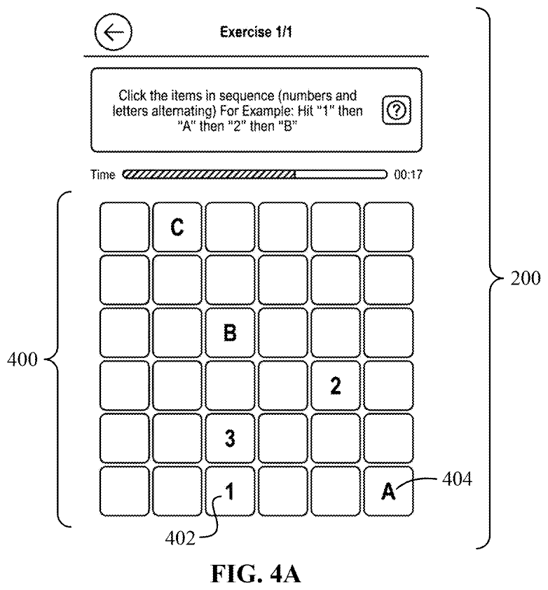

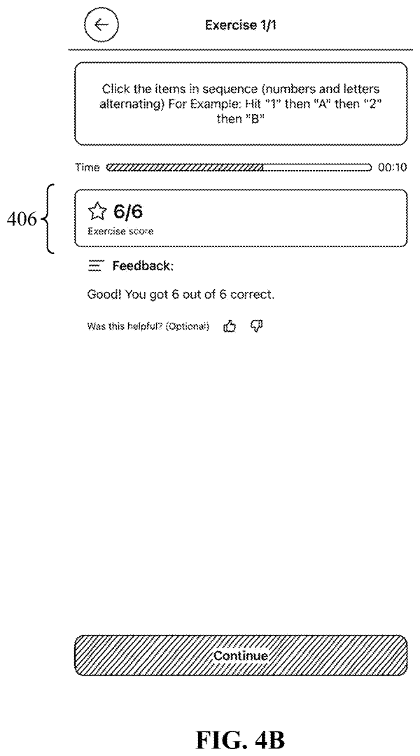

The system 100 executes a digitized cognitive testing module 220 on a mobile device 102, such as a smartphone or tablet. In one embodiment, the mobile device 102 presents an “Alternating Sequence Selection” test as shown in FIGS. 4A and 4B. During this assessment, the user 16a, 16b is presented with a spatial grid 400 comprising a plurality of randomized alphanumeric characters, such as numbers 402 (e.g., 1 through 3), and letters 404 (e.g., A through C). The user 16a, 16b is instructed via an instructional prompt 406 to interact with the graphical user interface by selecting the characters in an alternating, sequential order, such as “1”, then “A”, then “2”, then “B”. Traditionally, this assessment operates as a digitized embodiment of the clinical Trail Making Test Part B. Its primary clinical objective is to assess executive function, particularly cognitive flexibility and set shifting, by requiring the patient 16a, 16b to alternate between numerical and alphabetical sequences. The test actively evaluates attention, processing speed, and goal-directed behavior, wherein the user's brain must successfully hold and switch between two distinct logical rulesets.

To administer the baseline functional elements of this traditional test, the smartphone 102 relies on a minimum subset of basic hardware components. By way of example and not limitation, the device 102 requires a digital display screen to render the alphanumeric spatial grid 400, a standard capacitive touch sensor overlay to register that a user's finger has made contact with a specific predefined tile, and a basic system clock to initiate a countdown timer and record the total overall duration from the start of the exercise to its completion.

Utilizing these basic minimum sensors, the system 100 captures foreground interaction data to generate specific derived metrics. This is referred to as Layer 1 data. Upon the user 16a, 16b completing the test, or upon expiration of the timer, the smartphone 102 transmits via network 104 this raw foreground interaction data over a network to a remote cloud-based server 10. The server 10 processes this data to calculate a foreground task completion score (Layer 1) based on derived macroscopic metrics, including the total completion time, the number of incorrect selections, the longest correctly maintained alternating sequence, and a binary evaluation of sequence completion versus timeout. The server 10 then transmits via network 104 this calculated foreground score back to the smartphone 102, where the mobile device 102 updates its GUI screen 200 to display the score to the user 16a, 16b. Additionally, after the patient 16a, 16b has taken and completed the test, the mobile device 102 updates the module tracking interface, as illustrated in FIG. 2B, to reflect a newly earned checkmark along the visual pathway, providing the user 16a, 16b with immediate visual feedback of their progress and encouraging further daily interaction.

In various embodiments, the mobile device 102 functions primarily as a data acquisition and user interface platform, while computational evaluation of the test results is performed by the remote server 10. Accordingly, once the user completes a testing module, the mobile device transmits the captured foreground interaction data and background biometric sensor data to the server 10. The server executes one or more evaluation algorithms or machine learning models to interpret the data, generate performance scores, and extract neurological features or biomarkers. The resulting scores, classifications, or feedback are then transmitted back to the mobile device 102 for presentation to the user and are simultaneously stored within the clinician portal 12 for professional review.

Traditionally, clinicians 14 utilize these macroscopic metrics to diagnose broad cognitive impairments, diffuse cognitive slowing, or the progression of neurodegenerative diseases such as Alzheimer's disease, generalized dementia, or the cognitive sequelae of a Traumatic Brain Injury (TBI). In traditional longitudinal tracking, the patient 16a, 16b performs this test repeatedly over a period of months or years. The traditional marker indicating that a patient 16a, 16b is in neurological decline is a macroscopic “inflection point” or a downward trend in the foreground task score over a long period of time. For example, a clinician 14 reviewing the patient's file may note that the patient 16a, 16b historically completed the alternating sequence with high selection efficiency, such as a high volume of correct selections per second, and zero errors. However, if over the last three months the patient's total completion time has steadily degraded and the number of incorrect selections has increased, this macroscopic drop in overall performance is the traditional marker used to confirm that the patient's cognitive state is actively deteriorating.

However, modern smartphones 102 comprise a rich, multi-dimensional array of hardware sensors beyond the basic touch screen and timer. In an aspect, the present system 100 simultaneously captures background sensor data (Layer 2) while the user 16a, 16b is consciously focused on completing the foreground test. Background sensor data refers to sensor data captured by the smartphone 102 that is not required for execution of the specific cognitive testing module being administered at that time. As such, a particular sensor stream may function as foreground sensor data for one testing module and background sensor data for another testing module depending on the functional requirements of the module. When the user is interacting with non-clinical activities within the application, such as gamified exercises, sensor data that is not required for execution of those activities may be classified as background sensor data. Because the user's cognitive load is highly taxed by the foreground game, underlying motor and neurological deficits are unmasked and captured by the background sensors.

In certain embodiments, the measurable signals captured by the sensors of the mobile device 102 may be processed to derive digital neurological biomarkers. A digital neurological biomarker refers to a quantifiable behavioral or physiological measurement derived from sensor data that reflects neurological function or dysfunction. Examples of such biomarkers include tremor amplitude and dominant tremor frequency derived from motion sensor signals, reaction time variability derived from timestamped touchscreen interactions, sequencing error patterns derived from rule-based cognitive exercises, drawing coherence metrics derived from analysis of user-generated drawings, and psychomotor speed derived from inter-selection intervals during touchscreen interactions. These digital biomarkers may be evaluated individually or in combination by the server 10 to identify patterns associated with neurological health, neurological injury, or neurological disease progression.

In various implementations, the system 100 may conceptually associate each sensor with a measurable physical signal, a derived neurological feature or digital biomarker, and one or more neurological conditions that may be evaluated based on that biomarker. By way of example, an accelerometer may capture acceleration signals that are processed to derive tremor amplitude or tremor frequency characteristics, which may be associated with neurological conditions such as Parkinson's disease or essential tremor. Similarly, touchscreen interaction timestamps may generate reaction time signals that are processed to derive psychomotor speed or cognitive hesitation biomarkers associated with neurological decline or traumatic brain injury.

For example, while the user 16a, 16b holds the mobile device 102 to play the game, the system 100 continually polls background motion data from a multi-axis accelerometer and gyroscope to extract micro-oscillations and device sway including tremor amplitude and dominant tremor frequency characteristics, which are utilized to diagnose upper limb intention tremors. Signal processing of the motion sensor data may further extract tremor characteristics including tremor amplitude, dominant tremor frequency, tremor variability, and tremor stability across multiple testing sessions, which may assist in distinguishing between different tremor phenotypes including Parkinsonian tremor patterns, essential tremor patterns, and other motor impairment signatures. Concurrently, a high-resolution OS event logger provided by the native operating system securely timestamps every individual screen interaction. This enables the extraction of highly granular derived metrics, such as the exact time to the first correct selection and the mean inter-selection interval. By tracking the precise milliseconds between tapping one target and reaching for the next, the system 100 isolates psychomotor processing speed and detects sub-perceptual cognitive hesitation. Furthermore, the event logger tracks the exact timing of errors occurring at sequence switch points, such as transitioning from a number 402 to a letter 404 or a letter 404 to a number 402, thereby pinpointing specific set-shifting breakdowns. Additionally, a high-fidelity capacitive digitizer of the touch screen captures haptic pressure and maps the X-Y coordinates of the taps. This extracts a hemispatial interaction metric, allowing the system 100 to diagnose hemispatial neglect—a critical indicator of an acute ischemic stroke—if the sensor data reveals the user 16a, 16b takes significantly longer to strike targets on one side of the screen versus the other. In this regard, all data is background sensor data and categorized as a Layer 2 data.

Critically, this background data acquisition is not limited solely to the active testing phases of the clinical cognitive modules 220. In that case, the sensors needed for the test would be foreground sensor data. All the other sensor data would be background sensor data. The background data is actively gathered continuously whenever the application is open and the user 16a, 16b is interacting with the mobile device 102, even when the user 16a, 16b is simply playing one of the gamified cognitive tests 222. Furthermore, in certain embodiments, the mobile device 102 utilizes its integrated sensors to acquire this background biometric data continuously when the phone is powered on, even when the application itself is not actively open in the foreground.

Accordingly, the system 100 may capture sensor data under multiple operational states, including (i) during execution of clinical cognitive testing modules, (ii) during execution of gamified exercises or other application interactions, and (iii) during passive background monitoring when the mobile device is powered on. Data collected under these different operational contexts may be labeled or tagged to indicate the contextual activity during which the sensor data was acquired.

As the patient 16a, 16b interacts with the smartphone 102, this high-fidelity background sensor data is transmitted via network 104 to the remote cloud-based server 10 alongside the foreground data. The server 10 houses an Artificial Intelligence (AI) and Machine Learning (ML) architecture. Because relying on a macroscopic drop in the traditional foreground score is inherently reactive, the AI system is configured to identify a condition predictively, well before the traditional inflection point shows up in the foreground data. The processed results from the server 10 are transmitted to and rendered on a secure clinician portal 12. This clinician portal 12 provides the doctor with multi-dimensional data visualizations and, facilitates treatment plan recommendations based on the AI's analysis of the data that may be implemented on the patient.

To facilitate this capability, the AI system relies on supervised clinical flags. As patients 16a, 16b utilize the system 100 over time, some receive formal clinical diagnoses of neurological diseases from a physician. The diagnosis may result from the administration of the traditional alternating sequence test or other tests performed on the patient 16a, 16b either on paper or through the software app disclosed herein on the user's smartphone. This diagnosis is entered into the clinician portal 12 as a flag thereby setting a date of the diagnosis. The AI system utilizes these flags to train its neural networks to distinguish between the biometric signatures and performance patterns—derived from the background sensor data alone, the foreground task data alone, or a synergistic combination of the two—of normal, healthy brains and neurologically compromised brains. Additionally, patients 16a, 16b can be formally flagged within the clinician portal 12 as having no neurological disease after a baseline evaluation period of a predefined duration demonstrating stable health. Upon the clinician 14 setting this “normal” or “healthy” flag, the AI system looks back at the corresponding historical sensor data to model and strictly understand the biometric signatures of normal brains. By modeling these healthy baselines, the AI system becomes progressively better able to indicate whether a newly evaluated person is normal and will likely not develop a neurological disease in the future, contrasting them sharply against those who may develop a neurological disease.

The baseline evaluation period of a predefined duration refers to an initial monitoring phase during which the system 100 collects foreground interaction data and background biometric sensor data from the mobile device 102 while the user 16a, 16b interacts with the software application. During this period, the system establishes a personalized neurological baseline profile for the user based on longitudinal sensor data captured across multiple testing sessions and activities. The duration of the baseline evaluation period may vary depending on the clinical context and the amount of data required to characterize the user's typical neurological performance patterns. In certain circumstances, the baseline evaluation period may range from a relatively short observation window, such as a few hours or days of monitoring sufficient to rule out acute neurological abnormalities, to extended monitoring periods lasting months or years to establish long-term neurological stability or detect progressive neurological changes. Once the clinician 14 determines that the collected data demonstrates stable neurological behavior without clinically significant abnormalities, the clinician 14 may enter a corresponding normal or healthy flag within the clinician portal 12, thereby enabling the AI system to utilize the collected baseline data as a reference model for future comparisons.

This retrospective AI analysis is performed regularly and iteratively as more and more diagnoses and corresponding sensor data from a plurality of patients 16a, 16b are aggregated by the remote server 10. Upon receiving a new disease diagnosis flag, the AI system looks backward through that patient's historical sensor data, which may include the background sensor data alone, the foreground task data alone, or a combination of both, from the days, weeks or months prior to the clinical diagnosis, and preferably, prior to the macroscopic drop in their traditional foreground test score. The AI identifies hidden, multi-dimensional patterns—for example, a specific 4 Hz micro-tremor combined with a 12-millisecond increase in left-side mean inter-selection intervals—that occurred while the patient 16a, 16b was still scoring a perfect score on the foreground test. By doing so across a vast patient population, the system 100 may continuously learn and refine the early-warning signatures of the disease. As such, over time, the ability of the AI system to identify a potential diagnosis before traditional clinical methods may get progressively better. When the highly-trained AI detects this exact hidden pattern in the background and/or foreground sensor data of a new patient 16a, 16b, the clinician portal 12 may alerts the clinician 14 and suggests a treatment plan recommendation, such as prescribing a neuroprotective medication, preferably before the patient's conscious test scores (test scores from traditional cognitive tests) begin to indicate a decline.

Beyond early diagnosis, the present system 100 uniquely utilizes this dual-layer data architecture to facilitate evaluation of treatment efficacy. When a clinician 14 utilizes the clinician portal 12 to prescribe the recommended treatment protocol, such as a specific pharmacological agent or targeted cognitive therapy, the clinician 14 flags the initiation of this treatment plan within the portal 12.

Following the initiation of the treatment plan, the patient 16a, 16b continues to perform the digitized tests on their smartphone 102 repeatedly on a daily, weekly or monthly basis. Both the foreground task data and the background sensor data are continuously transmitted via network 104 to the remote server 10. Eventually, if the treatment is successful, the patient's clinical state improves, and the clinician 14 inputs a “getting better” or “treatment successful” diagnosis flag into the clinician portal 12.

The receipt of this positive outcome flag triggers a second retrospective AI analysis loop. The server's 10 AI system looks back at the continuous stream of data from the exact moment the “treatment prescribed” flag was set up moving forward in time until the “getting better” flag was entered. The AI analyzes the background sensor data, the foreground task data, or a combination of both layers, to identify the physiological and cognitive patterns indicating that the patient 16a, 16b was responding positively to the treatment. For example, the AI may determine that patients 16a, 16b who ultimately recover show a stabilization of haptic touch pressure and a smoothing of accelerometer micro-tremors within the first 72 hours of treatment, long before their macroscopic foreground game score or selection efficiency improves.

Similar to the diagnostic AI loop, this treatment efficacy analysis is performed regularly as outcome data from a plurality of patients 16a, 16b undergoing various treatment protocols is continuously gathered. As such, over time, the system's 100 ability to isolate the specific early-indicator patterns within the background sensor data, the foreground data, or a combination of the two, facilitates continuous improvement. Consequently, the application not only assists in identifying when a neurological problem will occur sooner, but its ability to identify whether a newly prescribed treatment plan may work earlier than traditional methods will get better over time. This predictive efficacy tracking allows the clinician 14 to dynamically adjust dosages or switch medications via the clinician portal 12 weeks or months earlier than traditional observational methods allow. If those physiological patterns do not show up early, then the clinician 14 can switch treatments sooner rather than wait for the traditional time allowed for the treatment to be deemed ineffective.

### Drawing Challenge

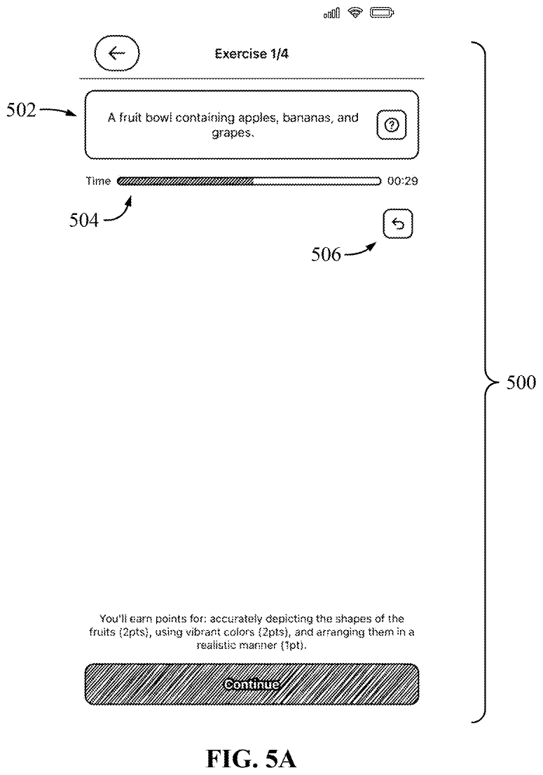

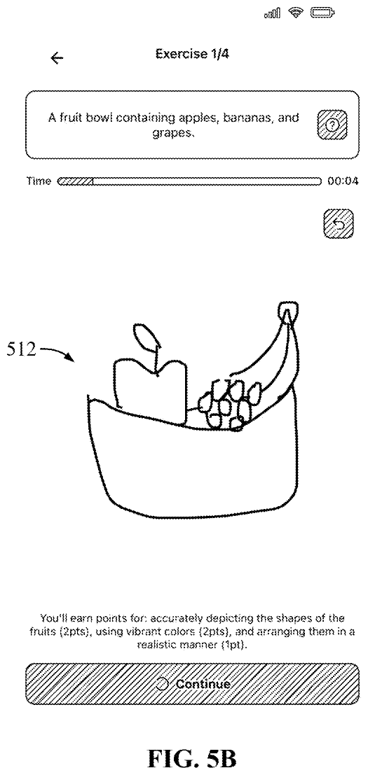

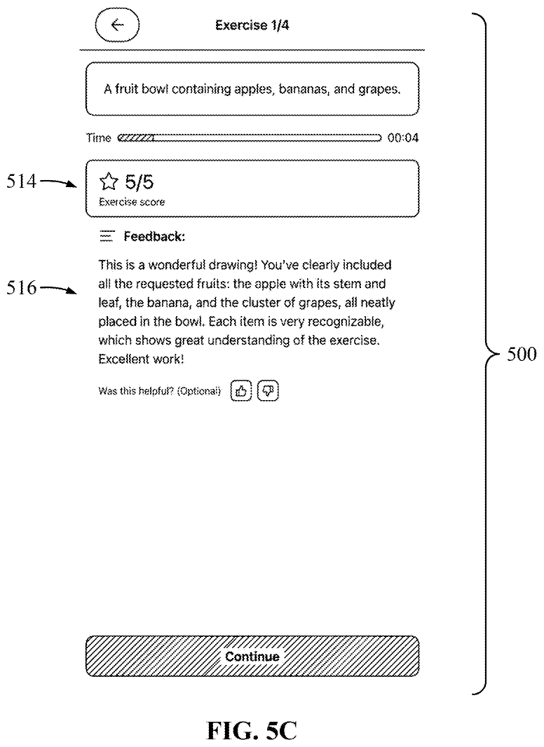

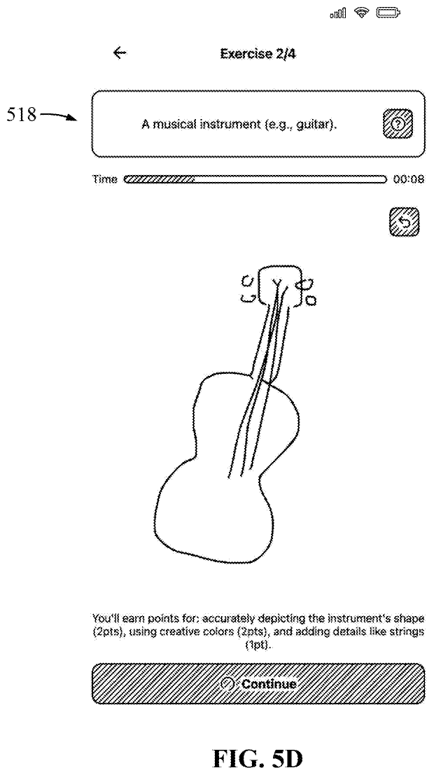

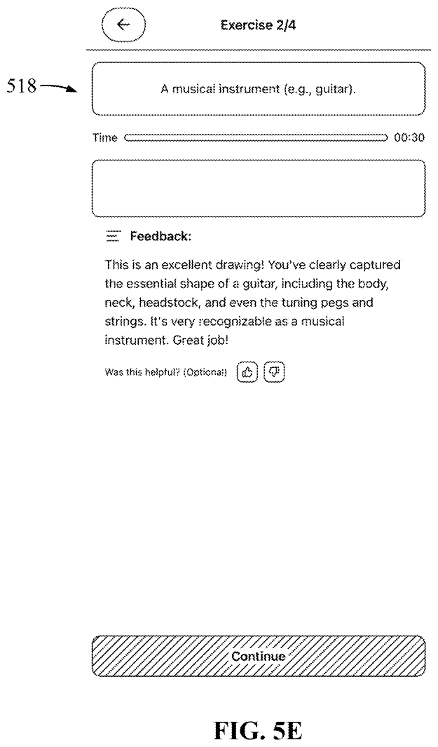

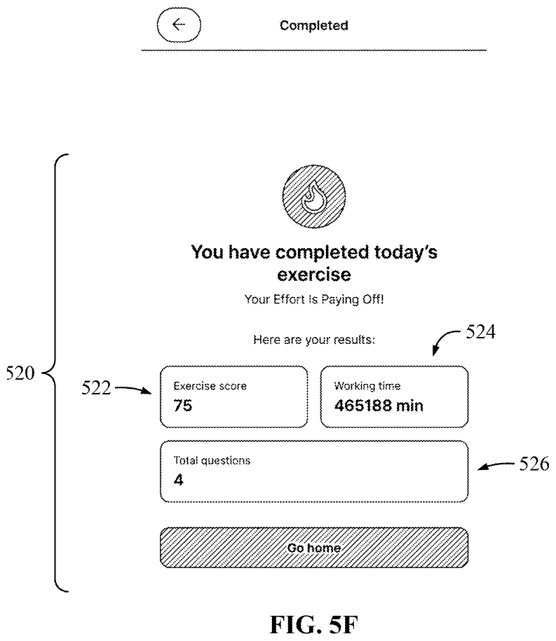

Referring now to FIGS. 5A-5F, an expressive visuospatial and fine motor assessment is shown. In particular, the mobile device 102 presents a “Drawing Challenge” test in which the user 16a, 16b is instructed to render a specific object or scene directly on the touchscreen interface. As shown in FIG. 5A, the graphical user interface 500 displays an instructional prompt 502, such as “A fruit bowl containing apples, bananas, and grapes,” along with a visual timer bar 504 and navigation controls 506. Upon presentation of the prompt 502, the system 100 initializes a time-stamped testing session. The mobile device 102 renders the instructions via its digital display and activates its capacitive multi-touch digitizer to capture user input. As the user begins drawing, the system captures foreground task data, including total working time, stroke count, stroke order, completion status, spatial arrangement of objects, and object inclusion metrics.

As illustrated in FIG. 5B, the user draws directly onto the screen, creating shapes 512 corresponding to the requested items. During this interaction, the mobile device 102 simultaneously captures high-resolution background sensor data while the user is cognitively engaged in the foreground task. Specifically, the device polls data from its multi-axis accelerometer and gyroscope to measure micro-oscillations, tremor frequency, and device sway while being held. The high-fidelity capacitive digitizer records X-Y coordinate trajectories, stroke velocity, stroke acceleration, touch pressure variation, and line smoothness. In addition, the operating system's event logger timestamps each stroke and inter-stroke pause with millisecond precision, enabling extraction of psychomotor latency, cognitive hesitation intervals, and motor planning irregularities. Thus, while the user consciously focuses on accurately drawing the fruit bowl, the system 100 unobtrusively collects a multidimensional biometric signature reflecting fine motor control, executive planning, visuospatial organization, and subclinical neurological irregularities.

Upon completion of the drawing, as illustrated in FIG. 5C, the mobile device 102 aggregates the raw foreground drawing data and the simultaneously collected background sensor data and securely transmits the data via network 104 to the remote cloud-based server 10. The transmission may occur in real time or in encrypted batched packets, depending on system configuration. The server 10 processes the received data using an Artificial Intelligence (AI) and Machine Learning (ML) architecture. The server executes image recognition models to determine whether the required elements—such as apples, bananas, and grapes—are present and recognizable. It evaluates structural proportions, spatial relationships, and object completeness to derive a visuospatial performance score. Simultaneously, the server analyzes motor-related features such as tremor frequency bands, pressure stability, stroke smoothness, and inter-stroke timing irregularities to derive fine motor control indices. Executive function metrics are further derived from stroke sequencing patterns and planning latency. Based on this multidimensional analysis, the server 10 calculates a foreground expressive cognition score 514 and additional derived neurological metrics. These processed results are then transmitted back via network 104 to the mobile device 102, which updates the graphical user interface 500 to display the score 514 and qualitative feedback 516 to the user 16a, 16b. They are also made accessible to the clinician via the clinician's portal 12.

In certain embodiments, the drawing module includes multiple sequential exercises within a single session. As shown in FIGS. 5D and 5E, the system 100 may present a subsequent prompt 518, such as “A musical instrument (e.g., guitar).” This second exercise further evaluates semantic memory retrieval, proportional reasoning, structural detail inclusion (e.g., strings, tuning pegs, neck alignment), and the user's ability to shift cognitive categories. As before, all foreground drawing metrics and background motion and touch data are captured and transmitted to the server 10 for AI-driven analysis. Even if the user receives a high macroscopic score 514, the AI system is configured to detect subtle deviations, such as increased tremor amplitude, delayed stroke initiation, hemispatial drawing bias, or reduced pressure consistency, which may represent early-stage neurological change not yet reflected in traditional scoring.

Upon completion of the exercise set, as illustrated in FIG. 5F, the mobile device 102 displays a consolidated summary screen 520 indicating an aggregate exercise score 522, total working time 524, and number of completed prompts 526. Concurrently, the server 10 stores the processed metrics within the user's profile. The clinician portal 12 is updated in near real time, allowing the clinician 14 to access multi-dimensional data visualizations. Within the portal 12, the clinician 14 may review time-series graphs of tremor frequency, stroke smoothness, inter-stroke latency distributions, hemispatial interaction asymmetry, and comparative baselines relative to age-matched normative populations.

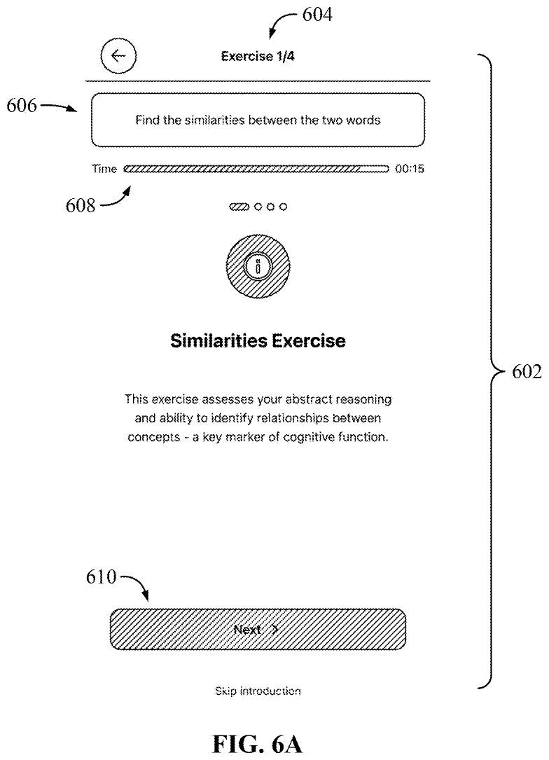

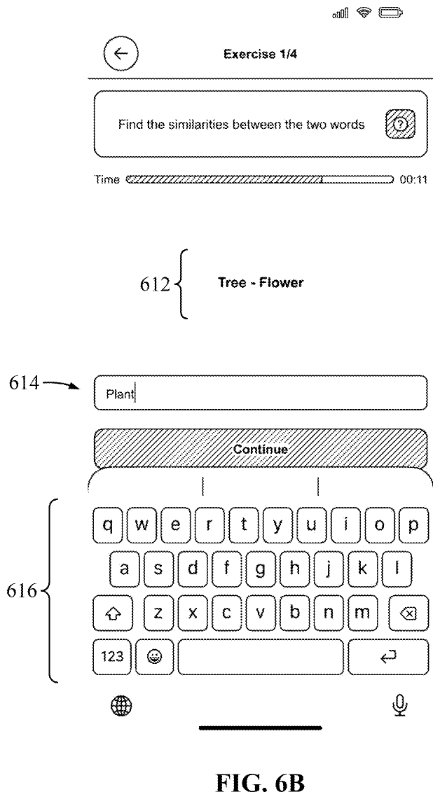

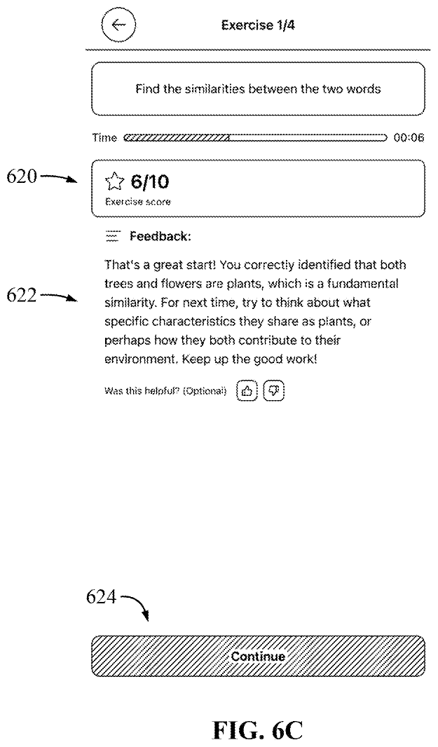

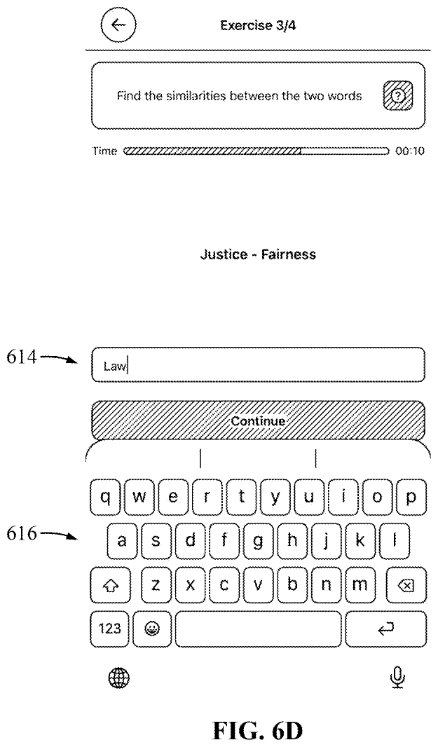

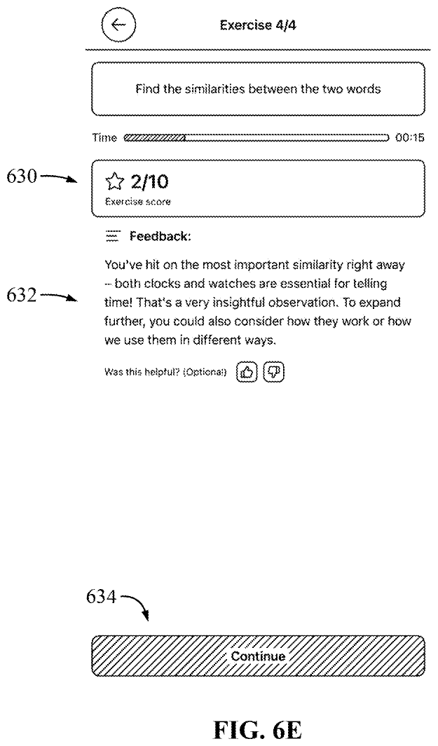

Referring now to FIGS. 6A-6E, the system 100 executes a Similarities Cognitive Test module 600 on the mobile device 102. The Similarities Cognitive Test module 600 evaluates abstract reasoning, conceptual categorization, semantic association, and executive cognitive function by requiring the user 16a, 16b to determine a conceptual relationship between two presented words. This exercise represents a digitized implementation of traditional similarities assessments commonly used in neurological and neuropsychological examinations to evaluate higher-level reasoning and the ability to identify relationships between concepts. The sequence of graphical interfaces illustrated in FIGS. 6A-6E depicts the various stages of the similarities testing module as presented to the user on the mobile device.

As illustrated in FIG. 6A, the graphical user interface 602 presented on the display of the mobile device 102 provides an introductory screen for the Similarities Cognitive Test module 600. The interface displays an exercise indicator 604 identifying the current exercise within the session, such as “Exercise 1/4.” An instructional prompt 606 informs the user that the objective of the exercise is to “Find the similarities between the two words.” A timer indicator 608 may be displayed to represent the time allotted for each exercise prompt. The interface may also include explanatory text describing that the exercise assesses abstract reasoning and the ability to identify relationships between concepts. A navigation button 610 may allow the user to proceed to the first question in the exercise sequence.

As illustrated in FIG. 6B, the mobile device 102 presents the first question in the exercise sequence. The graphical user interface 602 displays a pair of words 612 that the user must evaluate in order to determine their conceptual similarity. For example, the interface may display the word pair “Tree-Flower.” A response field 614 is provided to allow the user to enter the conceptual relationship between the two words. The user may enter a response using an on-screen keyboard 616 rendered on the touchscreen of the mobile device 102. During this interaction, the foreground sensors required to administer the test capture the primary task interaction data. These foreground sensors include the touchscreen capacitive digitizer of the mobile device 102, which detects keystroke inputs and records the spatial coordinates associated with each touch event on the virtual keyboard 616. The system clock functions as a timer that measures the duration between presentation of the word pair 612 and submission of the user's response, thereby enabling the system to determine the total response time required for the user to evaluate the conceptual relationship and enter the response.

As illustrated in FIG. 6C, after the user submits a response, the graphical interface transitions to a results interface displaying a performance score 620 for the exercise. The interface may display a feedback section 622 providing explanatory commentary regarding the conceptual similarity between the words. For example, the feedback may indicate that both trees and flowers are plants and therefore share a categorical relationship. A continuation button 624 may allow the user to proceed to the next exercise within the module.

As illustrated in FIG. 6D, the system may present additional word pairs in subsequent exercises to evaluate the user's ability to identify increasingly abstract conceptual relationships. For example, the interface may display a word pair such as “Justice-Fairness.” The user again enters a response into the response field 614 using the on-screen keyboard 616 while the mobile device records the associated interaction data and response timing characteristics.

As illustrated in FIG. 6E, the system may present additional results and feedback following the user's response to subsequent exercises. The interface may display a performance score 630 for the current exercise along with explanatory feedback text 632 acknowledging correct aspects of the response and suggesting additional conceptual relationships that could be identified. The interface may also provide a navigation button 634 allowing the user to proceed to additional exercises within the sequence.

While the user is actively interacting with the device to enter responses during the similarities exercise, the mobile device 102 simultaneously captures background biometric sensor data from sensors that are not required to administer the conceptual reasoning task but provide additional physiological and behavioral measurements. For example, motion sensors of the device, including a multi-axis accelerometer and gyroscope, may capture micro-movements of the device while the user holds or interacts with the phone. These signals may be analyzed to detect tremor amplitude, tremor frequency characteristics, and device sway patterns during interaction. Additionally, the touchscreen digitizer may capture touch pressure variations and subtle finger position adjustments during typing interactions. Because these signals are not required to administer the semantic reasoning task itself, they are categorized as background sensor data captured concurrently with the foreground interaction data.

After the user completes the exercise sequence, the mobile device 102 aggregates the captured foreground interaction data and the concurrently captured background biometric sensor data and transmits this data through network 104 to the remote server 10. The server 10 executes analytical algorithms, including artificial intelligence and machine learning models, to evaluate the received data. The server may derive cognitive performance metrics including conceptual reasoning accuracy, semantic association patterns, response latency distributions, typing hesitation intervals, and overall task completion time. Concurrently, background sensor data may be analyzed to derive neurological biomarkers including tremor characteristics derived from accelerometer signals and fine motor interaction patterns derived from touchscreen pressure and typing rhythm.

After the server 10 completes its analysis, the calculated similarities performance score and associated feedback are transmitted back via network 104 to the mobile device 102. The mobile device updates the graphical user interface 602 to display the score to the user. The analyzed results are also stored within the user's profile on the server and made accessible through the clinician portal 12. Within the clinician portal 12, the clinician 14 may review longitudinal performance metrics including conceptual reasoning accuracy, response timing distributions, lexical retrieval behavior, and associated motor interaction characteristics across repeated testing sessions. This information enables the clinician 14 to monitor cognitive health trends and detect early neurological impairments affecting executive reasoning, semantic association, or conceptual categorization abilities.

The sensors utilized in the Similarities Cognitive Test module include the touchscreen capacitive digitizer used to detect keystroke input and touchscreen interactions, the display screen used to render instructions and response interfaces, and the operating system event logger and system clock used to record high-resolution timestamps associated with user input events. Background sensors that may simultaneously capture behavioral and physiological signals include the multi-axis accelerometer for detecting device motion and tremor characteristics, the gyroscope for measuring device orientation and micro-movements during interaction, touchscreen pressure sensing capabilities that capture force and duration characteristics during typing interactions, and operating system interaction logs that record high-resolution timing patterns of user responses.

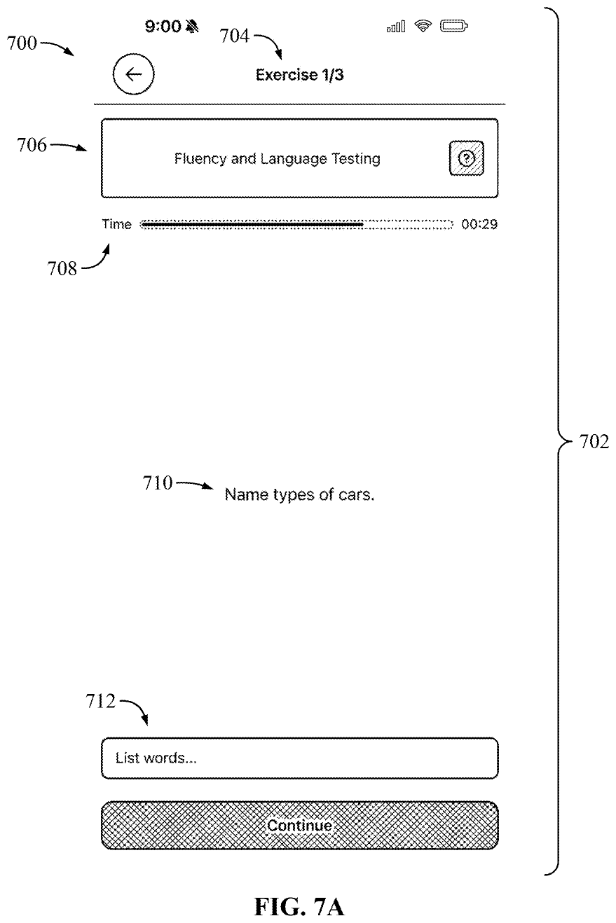

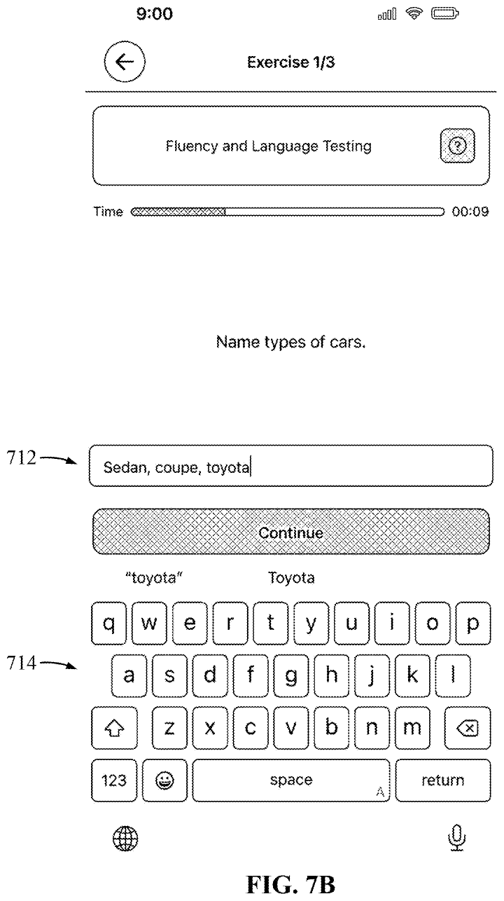

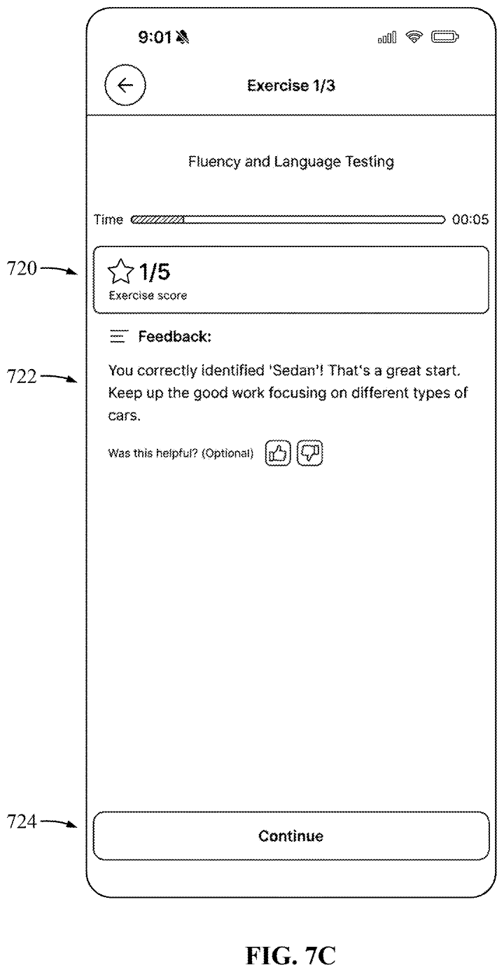

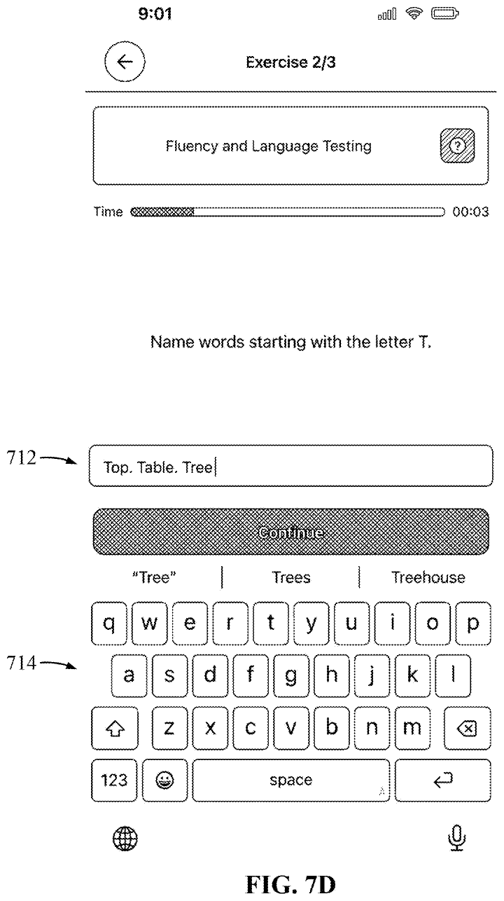

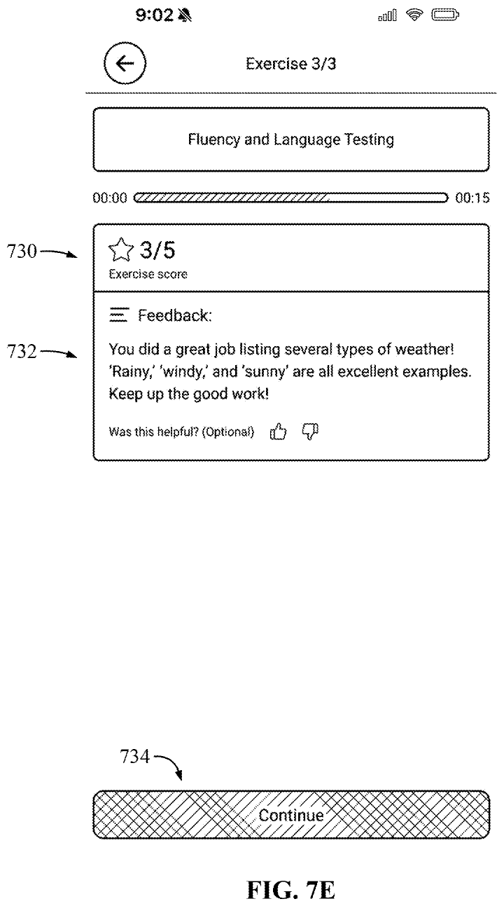

Referring now to FIGS. 7A-7E, the system 100 executes a Fluency and Language Testing module 700 on the mobile device 102. The Fluency and Language Testing module 700 evaluates verbal fluency, semantic memory retrieval, lexical access, and executive language processing by requiring the user 16a, 16b to generate multiple words that satisfy a specified semantic or phonemic constraint within a limited time period. This exercise represents a digitized implementation of traditional verbal fluency assessments commonly used in neurological and neuropsychological examinations to evaluate language production, cognitive flexibility, and semantic retrieval efficiency. The sequence of graphical interfaces illustrated in FIGS. 7A-7E depicts the stages of the fluency testing module as presented to the user on the mobile device.

As illustrated in FIG. 7A, the graphical user interface 702 presented on the display of the mobile device 102 provides an introductory screen for the Fluency and Language Testing module 700. The interface displays an exercise indicator 704 identifying the current exercise within the session, such as “Exercise 1/3.” An instructional prompt 706 identifies the exercise as a fluency and language task. A timer indicator 708 is displayed to indicate the amount of time available for the user to generate responses. The interface further presents a semantic prompt 710 instructing the user to generate words belonging to a particular category. In the illustrated example, the prompt instructs the user to “Name types of cars.” A response field 712 is provided for the user to enter generated words.

As illustrated in FIG. 7B, the user begins entering responses into the response field 712 using an on-screen keyboard 714 displayed on the touchscreen of the mobile device 102. For example, the user may enter responses such as “sedan,” “coupe,” or other vehicle types. Each keystroke entered by the user generates touchscreen interaction events captured by the mobile device. The foreground sensors required to administer the test include the touchscreen capacitive digitizer of the mobile device 102, which detects keystroke inputs and records the spatial coordinates associated with each touch event on the virtual keyboard 714. The system clock functions as a timer that measures the duration between presentation of the word pair 612 and submission of the user's response, thereby enabling the system to determine the total response time required for the user to evaluate the conceptual relationship and enter the response.

As illustrated in FIG. 7C, once the user completes the response period or the time limit expires, the graphical interface transitions to a results interface displaying a performance score 720 for the exercise. The interface may display feedback text 722 acknowledging correctly identified words and encouraging the user to generate additional responses in future exercises. The interface may also display a sequence of visual progress indicators representing cognitive development stages or learning steps. A continuation control 724 may allow the user to proceed to subsequent exercises within the module.

As illustrated in FIG. 7D, the system may present an additional fluency exercise in which the prompt requires the user to generate words satisfying a phonemic constraint. For example, the interface may instruct the user to “Name words starting with the letter T.” The user again enters responses into the response field 712 using the on-screen keyboard 714 while the mobile device records the associated interaction data and timing characteristics.

As illustrated in FIG. 7E, the system displays feedback after completion of the subsequent exercise. The interface may present a performance score 730 for the exercise and display explanatory feedback text 732 acknowledging correct responses, such as correctly identifying examples of weather conditions or other category-based responses. A continuation button 734 may allow the user to proceed to the next exercise within the sequence.

While the user is actively generating words during the fluency exercises, the mobile device 102 simultaneously captures background biometric sensor data from sensors that are not required to administer the language task but provide additional behavioral and physiological measurements. For example, motion sensors of the device, including a multi-axis accelerometer and gyroscope, may capture micro-movements of the device while the user holds or interacts with the phone. These signals may be analyzed to detect tremor amplitude, tremor frequency characteristics, and device sway patterns during interaction. Additionally, the touchscreen digitizer may capture touch pressure variations and subtle finger position adjustments during typing interactions. Because these signals are not required to administer the language exercise itself, they are categorized as background sensor data captured concurrently with the foreground interaction data.

After the user completes the exercise sequence, the mobile device 102 aggregates the captured foreground interaction data and the concurrently captured background biometric sensor data and transmits this data through network 104 to the remote server 10. The server 10 executes analytical algorithms, including artificial intelligence and machine learning models, to evaluate the received data. The server may derive verbal fluency performance metrics including the number of valid words generated, semantic clustering patterns, word generation rate, response latency between words, and hesitation intervals during the timed task. Concurrently, background sensor data may be analyzed to derive neurological biomarkers including tremor characteristics derived from accelerometer signals and fine motor interaction patterns derived from touchscreen pressure and typing rhythm.

After the server 10 completes its analysis, the calculated fluency performance score and associated feedback are transmitted back via network 104 to the mobile device 102. The mobile device updates the graphical user interface 702 to display the score to the user. The analyzed results are also stored within the user's profile on the server and made accessible through the clinician portal 12. Within the clinician portal 12, the clinician 14 may review longitudinal performance metrics including verbal fluency scores, word generation rates, response timing distributions, and associated motor interaction characteristics across repeated testing sessions. This information enables the clinician 14 to monitor cognitive health trends and detect early neurological impairments affecting language production, executive function, or semantic memory retrieval ability.

The sensors utilized in the Fluency and Language Testing module include the touchscreen capacitive digitizer used to detect keystroke input and touchscreen interactions, the display screen used to render prompts and response interfaces, and the operating system event logger and system clock used to record high-resolution timestamps associated with user input events. Background sensors that may simultaneously capture behavioral and physiological signals include the multi-axis accelerometer for detecting device motion and tremor characteristics, the gyroscope for measuring device orientation and micro-movements during interaction, touchscreen pressure sensing capabilities that capture force and duration characteristics during typing interactions, and operating system interaction logs that record high-resolution timing patterns of user responses.

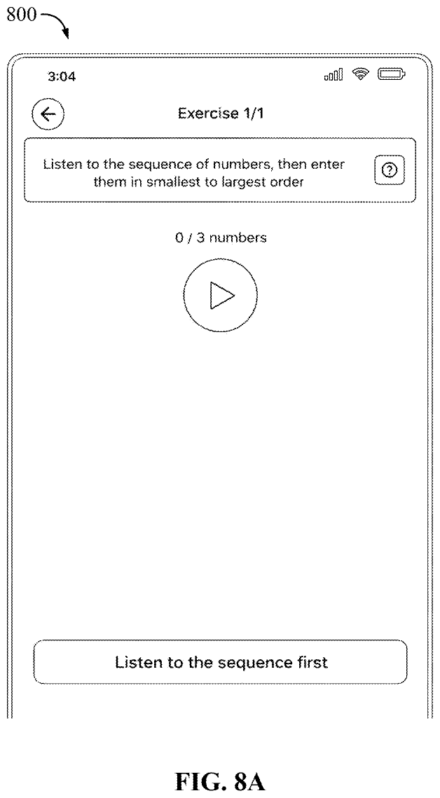

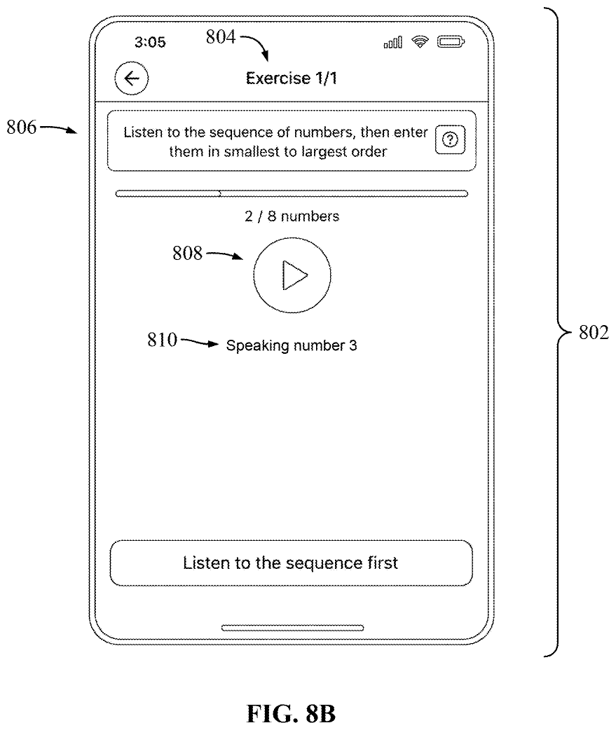

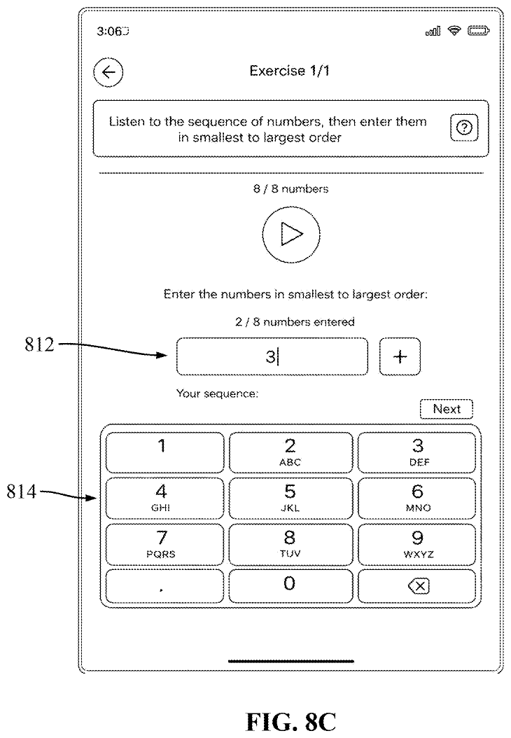

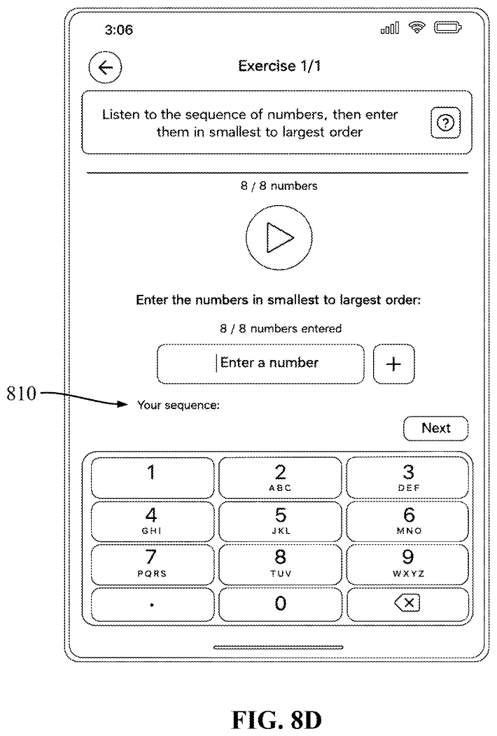

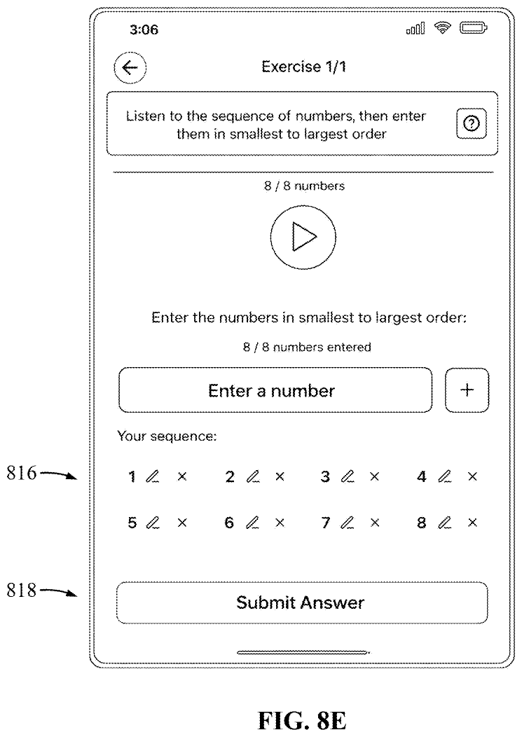

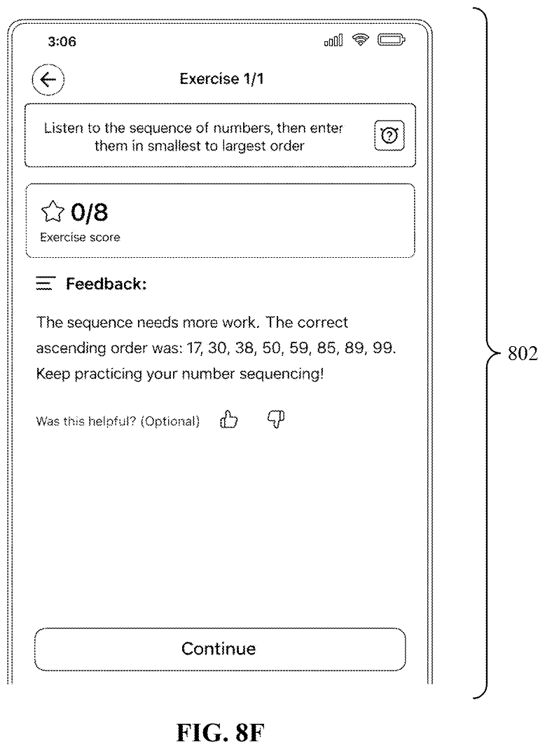

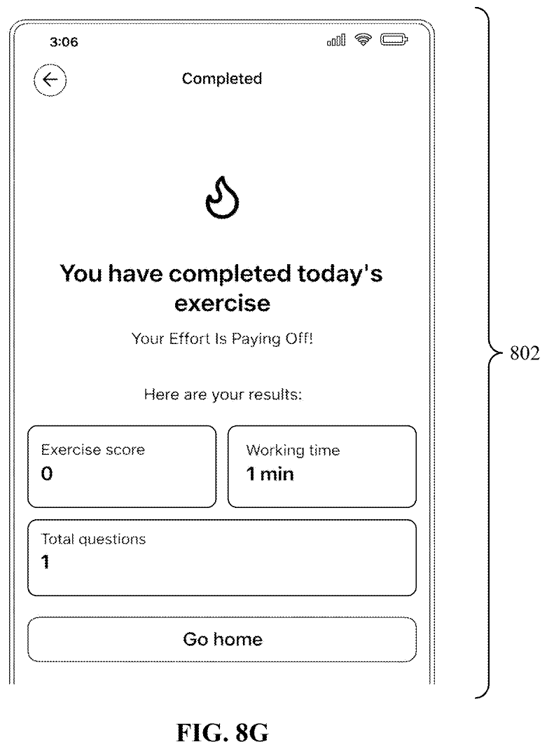

Referring now to FIGS. 8A-8G, the system 100 executes a Number Sequence Sorting Cognitive Test module 800 on the mobile device 102. The Number Sequence Sorting Cognitive Test module 800 evaluates auditory working memory, numerical reasoning, sequencing ability, and executive processing by requiring the user 16a, 16b to listen to a sequence of spoken numbers and then enter the numbers in ascending order using the touchscreen interface of the mobile device. This exercise represents a digitized implementation of traditional auditory sequencing and working-memory tasks used in neurological and neuropsychological examinations to evaluate executive function, attention, and short-term memory processing. The graphical interfaces illustrated in FIGS. 8A-8G depict the stages of the number sequence sorting module as presented to the user on the mobile device.

As illustrated in FIG. 8A, the graphical user interface 802 presented on the display of the mobile device 102 provides an introductory screen for the Number Sequence Sorting module 800. The interface displays an exercise indicator 804 identifying the current exercise, such as “Exercise 1/1.” An instructional prompt 806 informs the user to listen to a sequence of numbers and then enter the numbers in smallest-to-largest order. A playback control 808 is displayed that allows the user to initiate playback of the number sequence. A progress indicator 810 may also display the number of numbers presented during the sequence.

As illustrated in FIG. 8B, after the playback control is activated, the mobile device 102 sequentially outputs spoken numbers through the device speaker while visually indicating the progression of the sequence. For example, the interface may display a message such as “Speaking number 3” while the audio playback occurs. During this phase, the foreground components required to administer the test include the device speaker used to present the auditory number sequence and the system clock functioning as a timer to control the timing of the spoken numbers and measure the duration of the playback sequence.

After the spoken number sequence has been presented, the graphical interface transitions to an input interface as illustrated in FIG. 8C. The interface instructs the user to enter the numbers in smallest-to-largest order. A numeric entry field 812 is provided to allow the user to input each number of the sequence. A numeric keypad 814 rendered on the touchscreen allows the user to enter numbers directly into the input field. As each number is entered, the interface displays the partially reconstructed sequence.

As illustrated in FIG. 8D, the user continues entering the numbers from the sequence until all numbers have been entered. The interface may display a counter indicating how many numbers have been entered relative to the total number of numbers presented in the sequence. The reconstructed sequence is displayed in a sequence list 816 that visually represents the numbers entered by the user.

As illustrated in FIG. 8E, after the user has entered all numbers of the sequence, the interface provides a submission control 818 allowing the user to submit the reconstructed sequence for evaluation. The entered sequence is displayed in its entirety so that the user can review the input prior to submission.

During the interaction phase in which the user enters the sequence, the foreground sensors required to administer the test include the touchscreen capacitive digitizer of the mobile device 102, which detects numeric keypad selections and records the spatial coordinates associated with each touch event, and the system clock functioning as a timer that measures the duration between presentation of the number sequence and submission of the user's reconstructed sequence. The operating system event logger and system clock record high-resolution timestamps associated with each number entry. These inputs allow the system to derive response latency, inter-entry timing intervals, and overall task completion time.

As illustrated in FIG. 8F, once the sequence has been submitted, the graphical interface displays 802 a results screen indicating the user's exercise score. The interface may also provide explanatory feedback describing the correct ascending order of the numbers in the presented sequence. This feedback enables the user to understand the correct ordering and encourages continued engagement with the exercise.

As illustrated in FIG. 8G, the graphical user interface 802 displays a completion interface indicating that the user has completed the exercise session. The interface may display summary information including the exercise score, total working time, and the number of questions completed.

While the user is interacting with the device to enter the number sequence, the mobile device 102 simultaneously captures background biometric sensor data from sensors that are not required to administer the sequencing task but provide additional physiological and behavioral measurements. For example, motion sensors of the mobile device, including a multi-axis accelerometer and gyroscope, may capture micro-movements of the device while the user holds or interacts with the phone. These signals may be analyzed to detect tremor amplitude, tremor frequency characteristics, and device sway patterns during the interaction. Additionally, the touchscreen digitizer may capture touch pressure variations and subtle finger position adjustments during numeric keypad interactions. Because these signals are not required to administer the sequencing exercise itself, they are categorized as background sensor data captured concurrently with the foreground interaction data.

After the user completes the exercise, the mobile device 102 aggregates the captured foreground interaction data and the concurrently captured background biometric sensor data and transmits this data through network 104 to the remote server 10. The server 10 executes analytical algorithms, including artificial intelligence and machine learning models, to evaluate the received data. The server may derive cognitive performance metrics such as sequencing accuracy, ordering error patterns, response latency between number entries, and total completion time. Concurrently, background sensor data may be analyzed to derive neurological biomarkers including tremor characteristics derived from accelerometer signals and fine motor interaction patterns derived from touchscreen pressure and keypad interaction timing characteristics.

After the server 10 completes its analysis, the calculated sequencing performance score and associated feedback are transmitted back via network 104 to the mobile device 102. The mobile device updates the graphical user interface 802 to display the score to the user. The analyzed results are also stored within the user's profile on the server and made accessible through the clinician portal 12. Within the clinician portal 12, the clinician 14 may review longitudinal performance metrics including sequencing accuracy, auditory working memory performance, response timing distributions, and associated motor interaction characteristics across repeated testing sessions. This information enables the clinician 14 to monitor cognitive health trends and detect early neurological impairments affecting executive sequencing ability, auditory working memory retention, or cognitive processing speed.

The foreground components utilized in the Number Sequence Sorting Cognitive Test module include the device speaker used to present the auditory number sequence, the touchscreen capacitive digitizer used to detect numeric keypad input and touchscreen interactions, the display screen used to render instructions and number entry interfaces, and the system clock functioning as a timer to measure response duration during the exercise. Background sensors that may simultaneously capture behavioral and physiological signals include the multi-axis accelerometer for detecting device motion and tremor characteristics, the gyroscope for measuring device orientation and micro-movements during interaction, touchscreen pressure sensing capabilities that capture force and duration characteristics during keypad entry, and operating system interaction logs that record high-resolution timing patterns of user input.

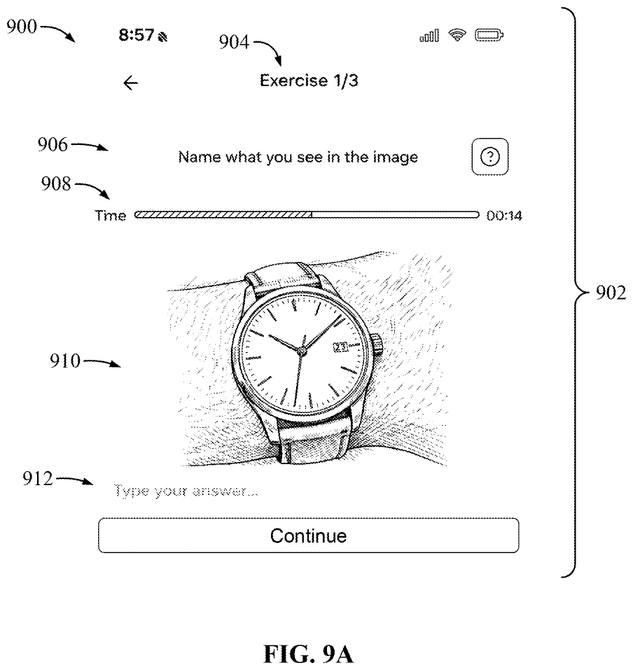

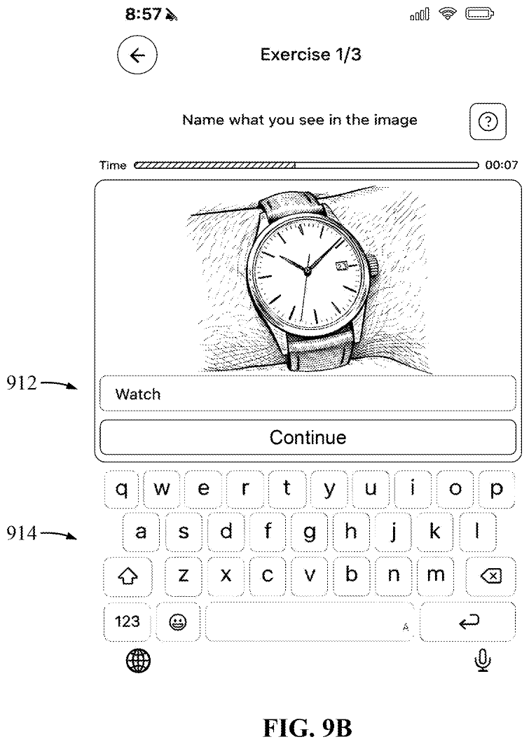

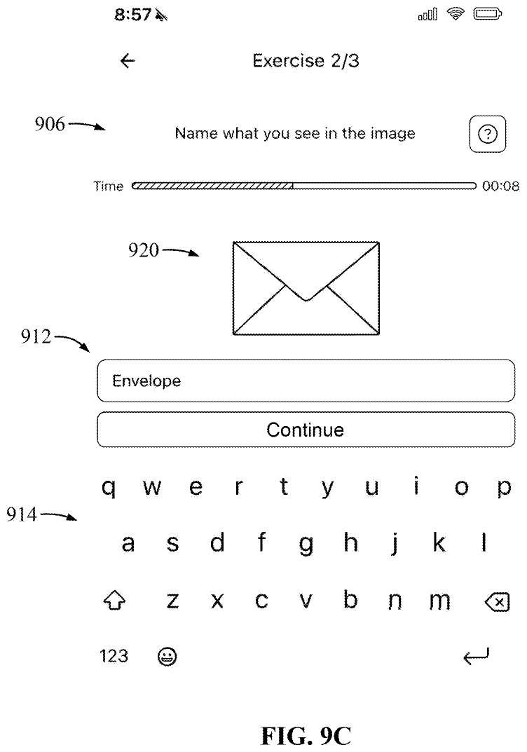

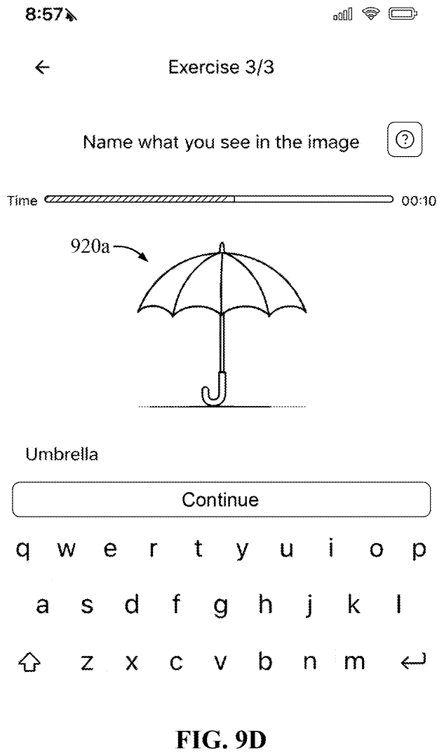

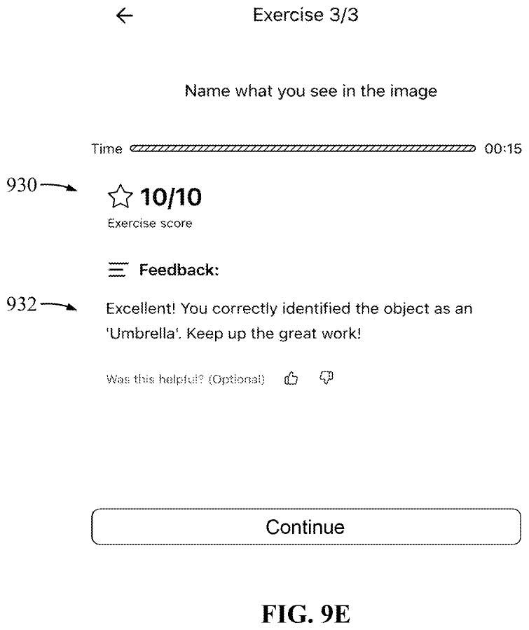

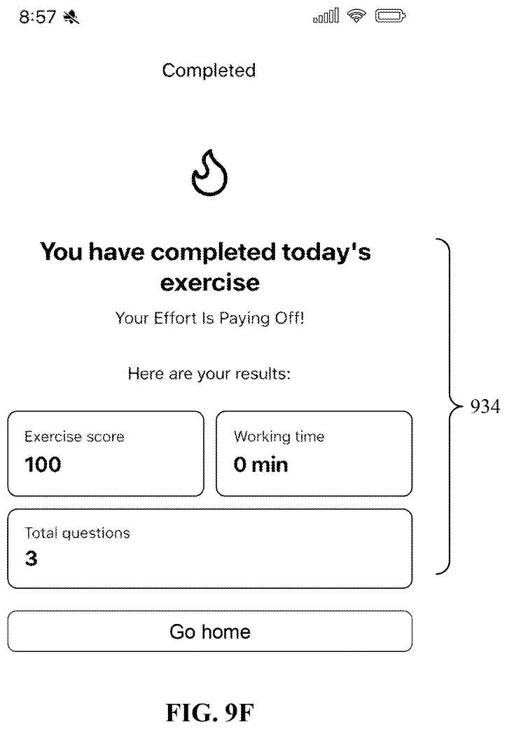

Referring now to FIGS. 9A-9F, the system 100 executes an Object Naming Cognitive Test module 900 on the mobile device 102. The Object Naming Cognitive Test module 900 evaluates visual recognition, semantic memory retrieval, lexical access, and language production by requiring the user 16a, 16b to identify and name objects presented in images displayed on the mobile device. This test represents a digitized implementation of traditional object naming assessments commonly used in neurological and neuropsychological examinations to evaluate language processing, visual perception, and word retrieval ability. The graphical interfaces illustrated in FIGS. 9A-9F depict the stages of the object naming exercise as presented to the user through the mobile device.

As illustrated in FIG. 9A, the graphical user interface 902 presented on the display of the mobile device 102 provides the initial object naming prompt. The interface displays an exercise indicator 904 identifying the current exercise within a sequence of exercises, such as “Exercise 1/3.” An instructional prompt 906 directs the user to “Name what you see in the image.” A timer indicator 908 displays the time remaining for the user to respond. The interface further presents an image 910 of an object—in this example, a wristwatch—displayed prominently within the screen. A response input field 912 is provided below the image to allow the user to enter the name of the object.

As illustrated in FIG. 9B, the user enters a response using an on-screen keyboard 914 displayed on the touchscreen of the mobile device 102. For example, the user may type “watch” into the response field 912. Each keystroke entered by the user generates touchscreen interaction events captured by the mobile device. The foreground sensors required to administer the test include the touchscreen capacitive digitizer of the mobile device 102, which detects keystroke inputs and records the spatial coordinates associated with each touch event on the virtual keyboard 914. The system clock functions as a timer that measures the duration between presentation of the object image and submission of the user's response, thereby enabling the system to determine the total response time required for the user to identify and enter the name of the object.

As illustrated in FIG. 9C, the system presents the next exercise in the sequence. The interface again displays the instructional prompt 906 and a new image 920. In this example, the image depicts an envelope. The user enters the corresponding object name into the response field 912 using the on-screen keyboard 914 while the mobile device records the associated interaction data and timing characteristics.

As illustrated in FIG. 9D, the system presents an additional object image 920a as part of the exercise sequence. In the illustrated example, the image depicts an umbrella. The user again enters the object name using the response field and keyboard interface. During each of these interactions, the mobile device captures foreground interaction data including keystroke sequences, response timing intervals, and overall response duration for each presented object.

While the user is actively interacting with the device to identify objects, the mobile device 102 simultaneously captures background biometric sensor data from sensors that are not required to administer the object naming task but provide additional physiological and behavioral measurements. For example, motion sensors of the mobile device, including a multi-axis accelerometer and gyroscope, may capture micro-movements of the device while the user holds or interacts with the phone. These signals may be analyzed to detect tremor amplitude, tremor frequency characteristics, and device sway patterns during interaction. Additionally, the touchscreen digitizer may capture touch pressure variations and subtle finger position adjustments during typing interactions. Because these signals are not required to administer the visual naming task itself, they are categorized as background sensor data captured concurrently with the foreground interaction data.

As illustrated in FIG. 9E, after the user completes the object identification exercise sequence, the interface displays a results screen presenting the user's exercise score 930 and explanatory feedback text 932 acknowledging the correct identification of the object. The interface may also provide optional feedback controls allowing the user to indicate whether the feedback was helpful.

As illustrated in FIG. 9F, the mobile device displays a completion interface indicating that the user has completed the exercise session. The interface may display summary information 934 including the exercise score, total working time, and the number of questions completed during the session.

At the conclusion of the exercise session, the mobile device 102 aggregates the captured foreground interaction data and the concurrently captured background biometric sensor data and transmits this information through network 104 to the remote server 10. The server 10 executes analytical algorithms, including artificial intelligence and machine learning models, to evaluate the received data. The server may derive cognitive performance metrics including object recognition accuracy, lexical retrieval speed, response latency distributions, and typing hesitation patterns. Concurrently, background sensor data may be analyzed to derive neurological biomarkers including tremor characteristics derived from accelerometer signals and fine motor interaction patterns derived from touchscreen pressure and keystroke timing characteristics.

After the server 10 completes its analysis, the calculated object naming performance score and associated feedback are transmitted back via network 104 to the mobile device 102. The mobile device updates the graphical user interface to display the score to the user. The analyzed results are also stored within the user's profile on the server and made accessible through the clinician portal 12. Within the clinician portal 12, the clinician 14 may review longitudinal performance metrics including object recognition accuracy, lexical retrieval timing, response latency distributions, and associated motor interaction characteristics across repeated testing sessions. This information enables the clinician 14 to monitor cognitive health trends and detect early neurological impairments affecting visual recognition, language production, or semantic memory retrieval.

The sensors utilized in the Object Naming Cognitive Test module include the touchscreen capacitive digitizer used to detect keystroke input and touchscreen interactions, the display screen used to render object images and response interfaces, and the operating system event logger and system clock used to record high-resolution timestamps associated with user input events. Background sensors that may simultaneously capture behavioral and physiological signals include the multi-axis accelerometer for detecting device motion and tremor characteristics, the gyroscope for measuring device orientation and micro-movements during interaction, touchscreen pressure sensing capabilities that capture force and duration characteristics during typing interactions, and operating system interaction logs that record high-resolution timing patterns of user responses.

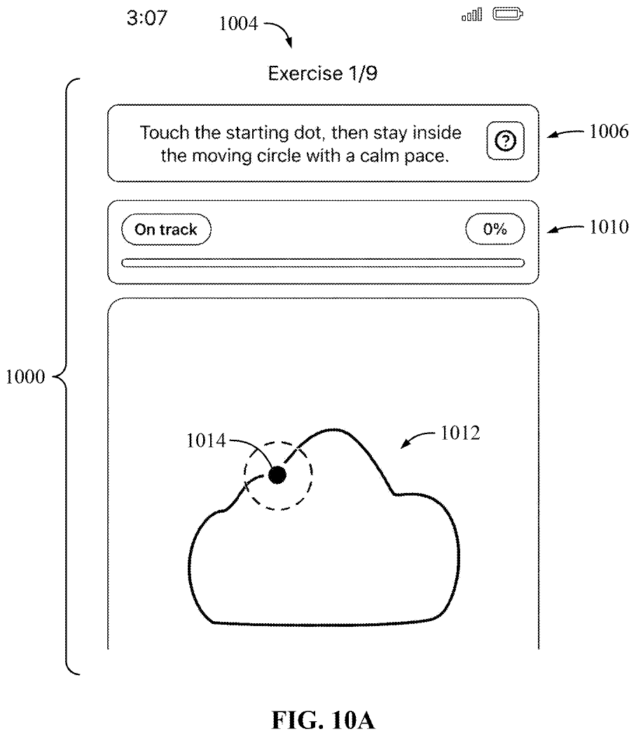

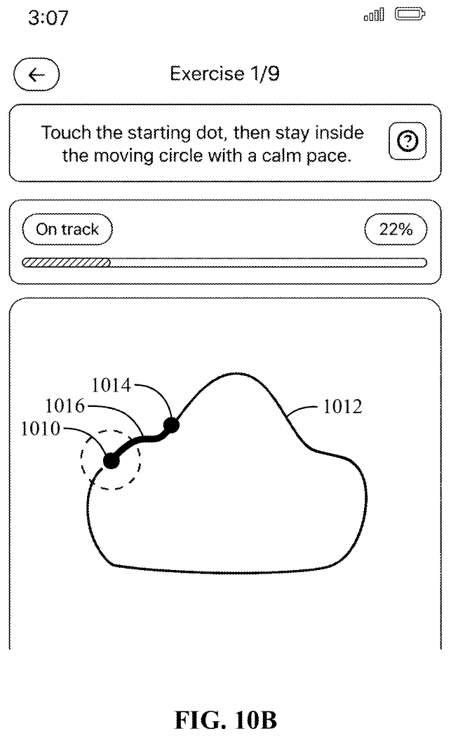

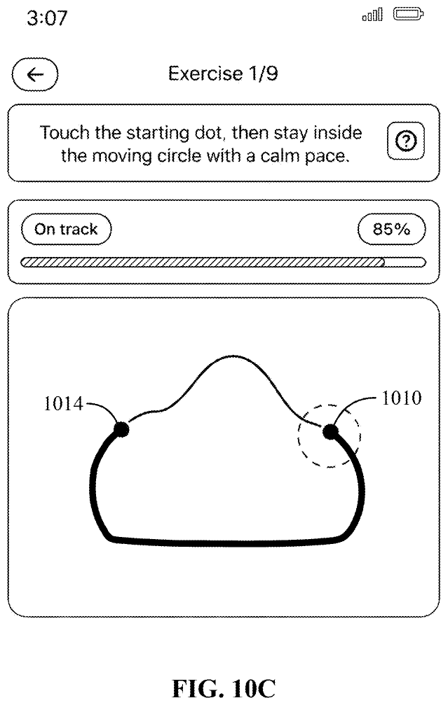

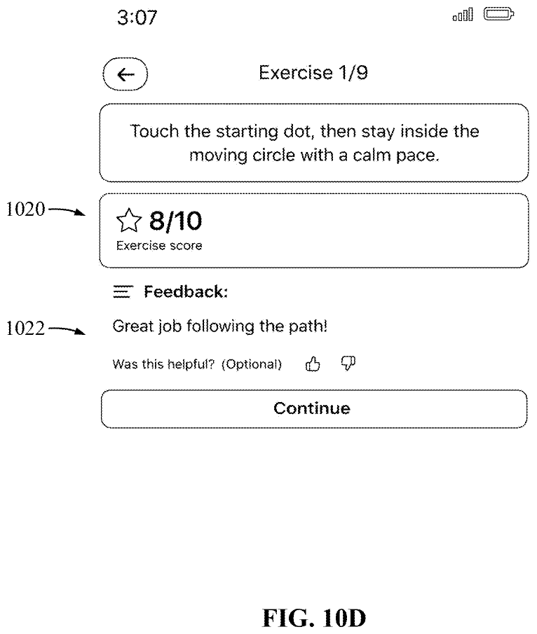

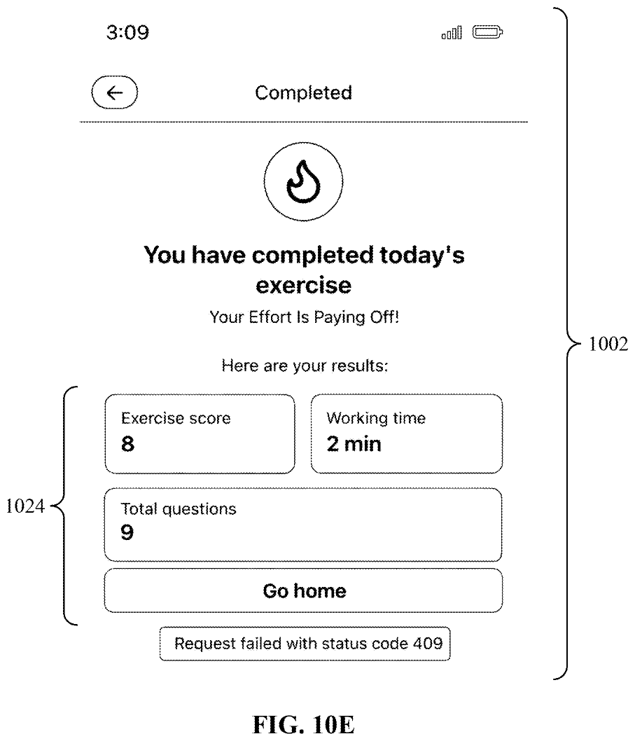

Referring now to FIGS. 10A-10E, the system 100 executes a Path Following Cognitive Test module 1000 on the mobile device 102. The Path Following Cognitive Test module 1000 evaluates visuomotor coordination, fine motor control, attention stability, and executive motor planning by requiring the user 16a, 16b to trace a predefined path displayed on the touchscreen while maintaining alignment with a moving indicator. This exercise represents a digitized implementation of traditional motor tracing and coordination tests used in neurological and neuropsychological evaluations to assess hand-eye coordination, motor precision, and the ability to sustain controlled movement along a defined trajectory. The graphical interfaces illustrated in FIGS. 10A-10E depict the stages of the path-following exercise as presented to the user through the mobile device.

As illustrated in FIG. 10A, the graphical user interface 1002 presented on the display of the mobile device 102 provides the initial instructions for the path-following task. The interface displays an exercise indicator 1004 identifying the current exercise within a sequence of exercises, such as “Exercise 1/9.” An instructional prompt 1006 directs the user to touch a starting dot and then remain inside a moving circular indicator while tracing the path at a calm pace. The interface further displays a status indicator 1008 indicating whether the user is “On track,” along with a progress indicator 1010 initially displaying zero percent completion. A predefined path outline 1012 is displayed on the screen together with a starting point 1014 indicating where the user should begin the tracing movement.

As illustrated in FIG. 10B, the user begins tracing the path by touching the starting point 1014 and moving a finger along the displayed path outline 1012. The graphical interface displays a moving circular indicator surrounding the user's finger position and updates the progress indicator 1010 as the user advances along the path. The traced trajectory 1016 generated by the user's finger movement becomes visible along the path. During this interaction, the foreground sensors required to administer the test include the touchscreen capacitive digitizer of the mobile device 102, which continuously records the X-Y coordinate trajectory of the user's finger as it moves across the screen. The touchscreen digitizer also captures movement velocity, stroke acceleration, and spatial deviation of the traced path relative to the predefined path outline.

As illustrated in FIG. 10C, the user continues tracing the path while attempting to remain within the moving circular indicator. The graphical interface updates the progress indicator 1010 to reflect the percentage of the path completed, such as 85 percent completion shown in the illustrated example. The system clock functions as a timer that measures the duration of the tracing activity from the moment the user begins tracing at the starting point 1014 until the tracing path is completed, thereby enabling the system to determine the total time required to complete the path-following task. The operating system event logger and system clock record high-resolution timestamps associated with each touch movement event, allowing the system to derive metrics including tracing duration, movement smoothness, deviation frequency, and pause intervals during the tracing motion.

While the user performs the tracing activity, the mobile device 102 simultaneously captures background biometric sensor data from sensors that are not required to administer the tracing task but provide additional physiological and behavioral measurements. For example, motion sensors of the mobile device, including a multi-axis accelerometer and gyroscope, capture micro-movements of the device while the user holds or interacts with the phone. These signals may be analyzed to detect tremor amplitude, tremor frequency characteristics, and device sway patterns during the tracing activity. Additionally, the touchscreen digitizer may capture touch pressure variation and subtle finger stability characteristics during the tracing movement. Because these signals are not required to administer the path-following exercise itself, they are categorized as background sensor data captured concurrently with the foreground interaction data.

As illustrated in FIG. 10D, once the user completes the tracing path, the graphical interface transitions to a results screen displaying a performance score 1020 for the exercise. The interface may also present feedback text 1022 acknowledging successful path tracking and encouraging continued participation in the exercise sequence.

As illustrated in FIG. 10E, the mobile device displays a completion interface indicating that the user has finished the exercise session. The interface may display summary information 1024 including the exercise score, working time, and the total number of questions or exercises completed during the session.

At the conclusion of the exercise session, the mobile device 102 aggregates the captured foreground interaction data and the concurrently captured background biometric sensor data and transmits this information through network 104 to the remote server 10. The server 10 executes analytical algorithms, including artificial intelligence and machine learning models, to evaluate the received data. The server may derive cognitive performance metrics including path adherence accuracy, spatial deviation patterns, tracing smoothness, stroke velocity variability, and total task completion time. Concurrently, background sensor data may be analyzed to derive neurological biomarkers including tremor characteristics derived from accelerometer signals and motor stability indicators derived from touchscreen pressure variation and movement consistency.

After the server 10 completes its analysis, the calculated path-following performance score and associated feedback are transmitted back via network 104 to the mobile device 102. The mobile device updates the graphical user interface 1002 to display the score to the user. The analyzed results are also stored within the user's profile on the server and made accessible through the clinician portal 12. Within the clinician portal 12, the clinician 14 may review longitudinal performance metrics including path adherence accuracy, movement smoothness, tracing deviation patterns, and associated motor interaction characteristics across repeated testing sessions. This information enables the clinician 14 to monitor cognitive health trends and detect early neurological impairments affecting fine motor control, visuomotor coordination, or motor planning ability.

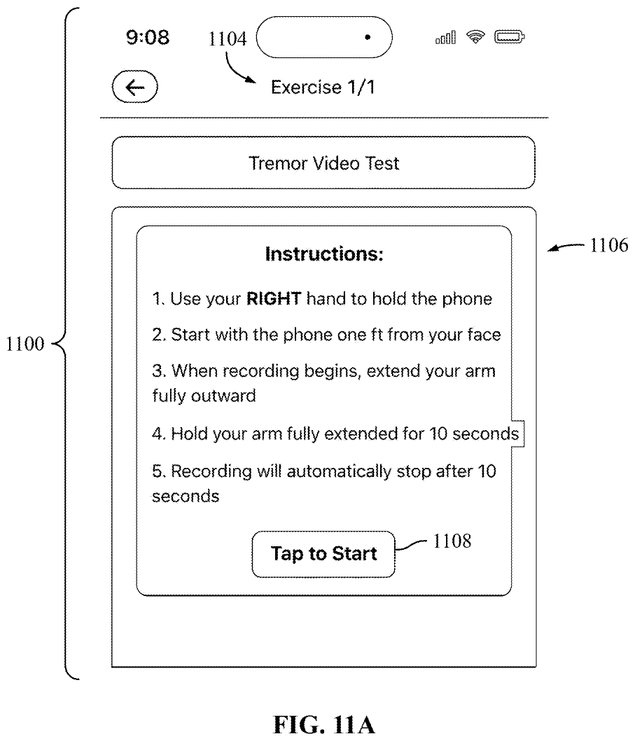

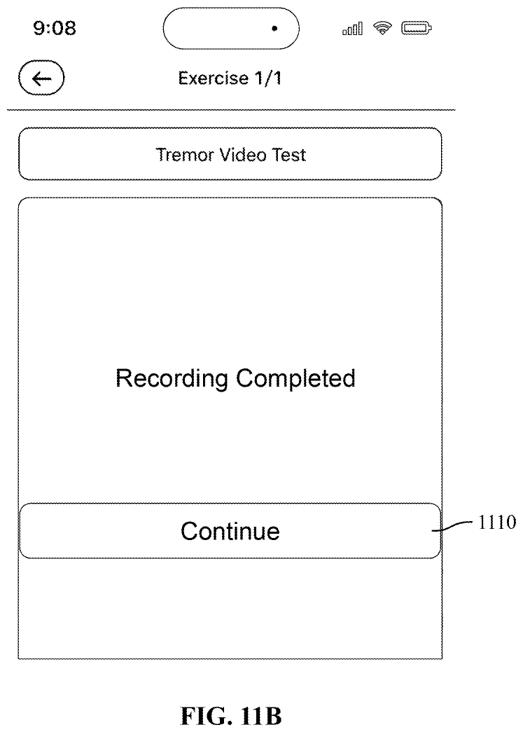

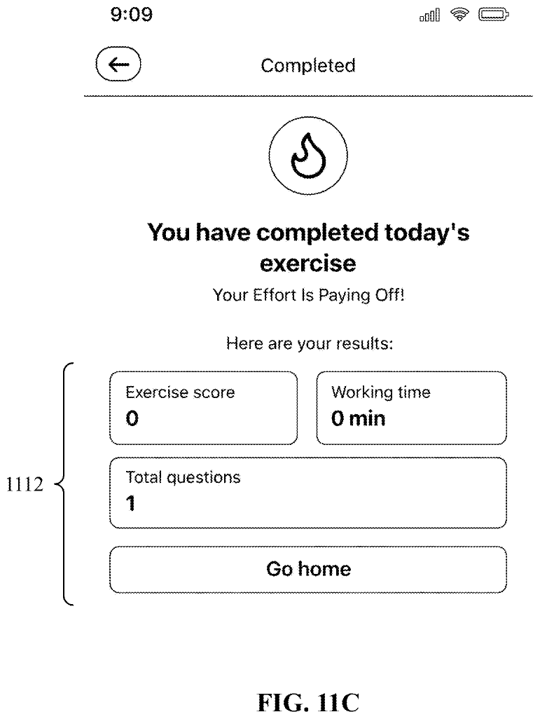

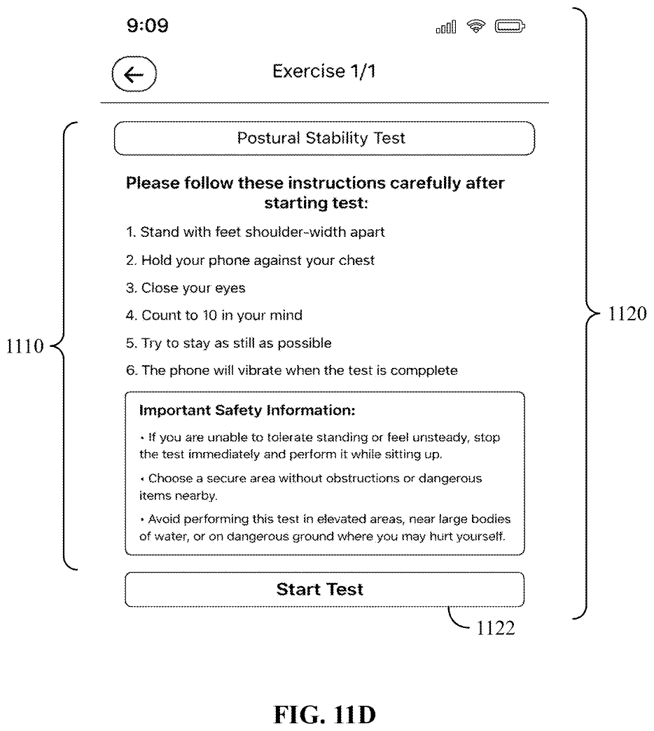

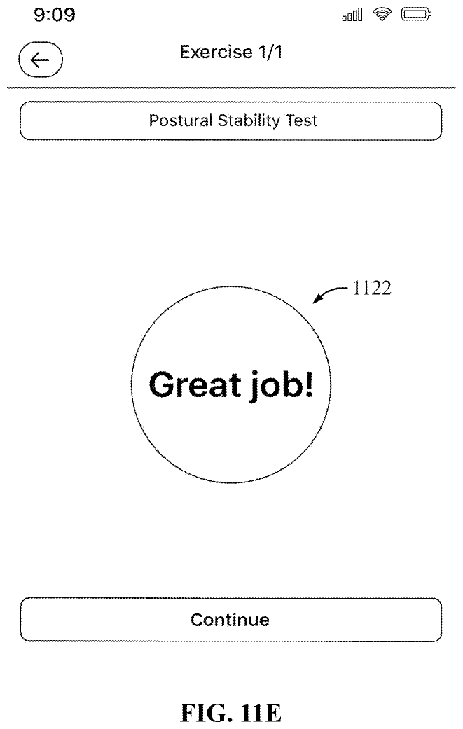

Referring now to FIGS. 11A-11E, the system 100 executes a Postural Stability and Tremor Assessment module 1100 on the mobile device 102. The Postural Stability and Tremor Assessment module 1100 evaluates upper-limb tremor characteristics, postural stability, and involuntary motor oscillations by utilizing the motion sensors and camera of the mobile device while the user performs a controlled posture task. This test represents a digitized implementation of neurological tremor and postural stability evaluations commonly performed during clinical examinations for neurological conditions such as Parkinson's disease, essential tremor, or other motor control disorders. The graphical interfaces illustrated in FIGS. 11A-11E depict the stages of the posture and tremor testing module as presented to the user through the mobile device.

As illustrated in FIG. 11A, the graphical user interface 1102 presented on the display of the mobile device 102 provides an introductory screen for the tremor video test. The interface displays an exercise indicator 1104 identifying the current exercise within the session, such as “Exercise 1/1.” An instructional panel 1106 presents step-by-step instructions directing the user to hold the phone with their right hand, begin with the device approximately one foot from the face, extend the arm fully outward when recording begins, and hold the arm in the extended position for a predetermined duration, such as ten seconds. A start control 1108 labeled “Tap to Start” allows the user to initiate the recording process.

When the user activates the start control 1108, the mobile device initiates a recording session as illustrated in FIG. 11B. Upon completion, a continue control 1110 is presented. During this recording phase, the mobile device may activate one or more onboard sensors to capture motion data and visual data associated with the user's arm position and movement stability. The foreground sensors used to administer this test include the device camera, which records video of the arm and device orientation during the test interval, the multi-axis accelerometer and gyroscope that capture motion signals representing tremor amplitude and frequency while the arm remains extended, and the system clock functioning as a timer to measure the duration of the recording interval. In certain embodiments, the motion sensors of the mobile device, including a multi-axis accelerometer and gyroscope, function as foreground sensors because they are directly required to capture motion signals representing tremor amplitude and frequency while the arm remains extended.

As illustrated in FIG. 11C, after the recording interval concludes, the mobile device displays a completion interface indicating that the exercise has finished. The interface may display summary information 1112 indicating that the exercise session has been completed and may present basic metrics such as working time or exercise status.

In certain embodiments, the system may also provide a second related test illustrated in FIGS. 11D and 11E, referred to as a Postural Stability Test module 1110. As illustrated in FIG. 11D, the graphical user interface 1120 presents instructions directing the user to stand with feet shoulder-width apart, hold the mobile device against the chest, close their eyes, and remain as still as possible for a short measurement interval. The interface may also present safety information advising the user to stop the test if they feel unstable or to perform the test in a safe environment. A start control 1122 allows the user to initiate the postural stability measurement.

During execution of the postural stability test, the foreground sensors used to administer the test include the multi-axis accelerometer and gyroscope of the mobile device 102, which capture device movement signals while the device is held against the user's chest. These sensors measure subtle body sway patterns, balance adjustments, and posture oscillations while the user attempts to remain stationary. The system clock functions as a timer that measures the duration of the measurement interval while motion data from the accelerometer and gyroscope is collected during the stability assessment.

While the tremor and postural stability exercises are being performed, the mobile device 102 simultaneously captures background biometric sensor data from sensors that are not required to administer the core task but provide additional physiological and behavioral measurements. For example, touchscreen interaction sensors may capture incidental touch events or pressure signals when the user holds the device. In addition, the device microphone may capture ambient audio data that can be analyzed to determine environmental context during testing. Because these sensor streams are not required to administer the tremor or posture test itself, they are categorized as background sensor data captured concurrently with the foreground sensor data.

After completion of the tremor or posture measurement interval, a completion message 1122 is shown (see FIG. 11E). Also, the mobile device 102 aggregates the captured foreground motion and video data and the concurrently captured background biometric sensor data and transmits this information through network 104 to the remote server 10. The server 10 executes analytical algorithms, including artificial intelligence and machine learning models, to evaluate the received data. The server may derive neurological performance metrics including tremor amplitude, dominant tremor frequency bands, tremor variability, arm stability characteristics, and body sway patterns during the measurement interval. In certain embodiments, video analysis algorithms may also evaluate the captured video stream to identify visual indicators of tremor, arm drift, or posture instability.

After the server 10 completes its analysis, the calculated tremor and postural stability metrics may be transmitted back via network 104 to the mobile device 102. The mobile device updates the graphical user interface to provide feedback to the user indicating completion of the exercise. The analyzed results are also stored within the user's profile on the server and made accessible through the clinician portal 12. Within the clinician portal 12, the clinician 14 may review longitudinal tremor measurements, body sway metrics, and stability indicators collected across repeated testing sessions. These measurements enable the clinician 14 to monitor neurological health trends and detect early motor impairments associated with neurodegenerative disorders, movement disorders, or balance impairments.

The foreground sensors utilized in the Postural Stability and Tremor Assessment module include the multi-axis accelerometer used to capture motion acceleration signals representing tremor amplitude and body sway characteristics, the gyroscope used to measure rotational movement and device orientation changes during the test interval, the device camera used to record video of the arm extension during the tremor video test, and the system clock functioning as a timer to measure the duration of the measurement interval. Background sensors that may simultaneously capture behavioral and environmental signals include the touchscreen pressure sensor used to detect incidental touch interaction patterns and the device microphone used to capture environmental audio signals during the testing session.

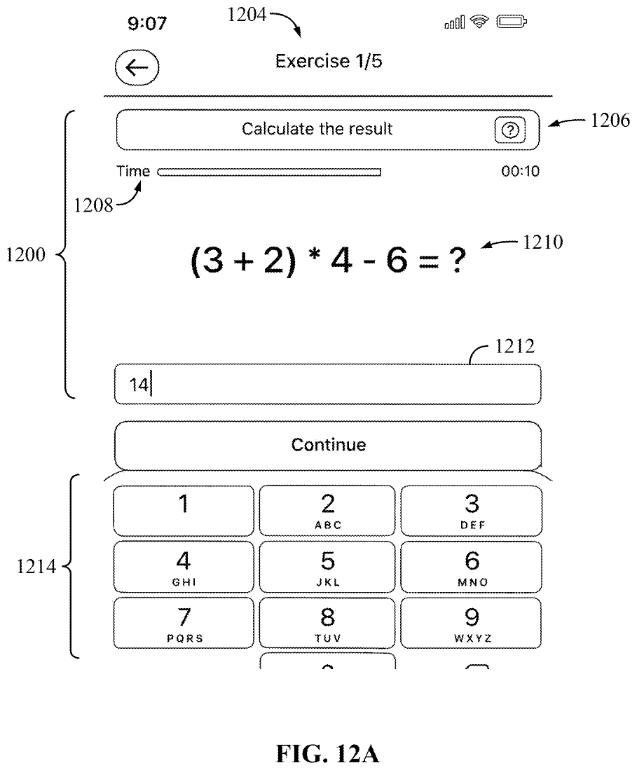

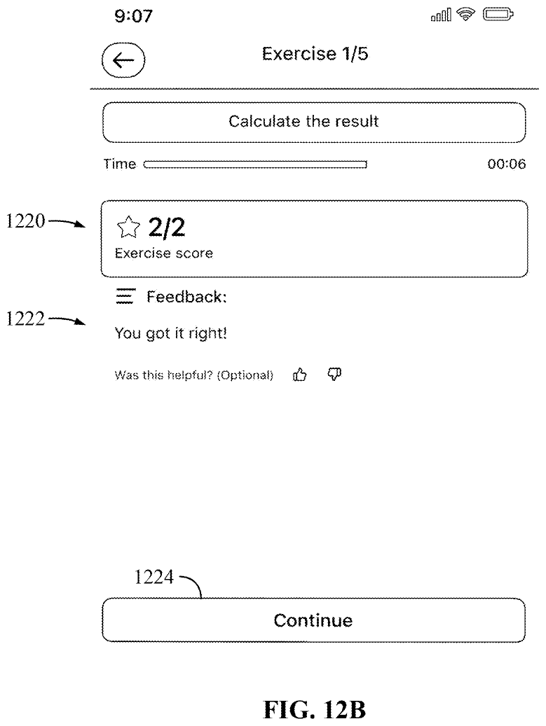

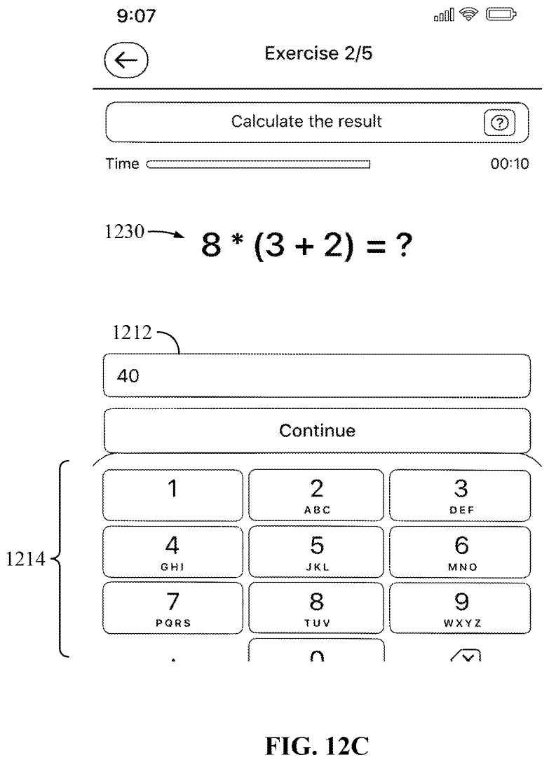

Referring now to FIGS. 12A-12C, the system 100 executes a Quick Math Cognitive Test module 1200 on the mobile device 102. The Quick Math Cognitive Test module 1200 evaluates numerical reasoning, working memory, attention, and cognitive processing speed by requiring the user 16a, 16b to mentally calculate the result of a mathematical expression and enter the correct answer within a limited time period. This exercise represents a digitized implementation of traditional mental arithmetic tests used in neurological and neuropsychological examinations to evaluate executive function, concentration, and cognitive processing efficiency. The graphical interfaces illustrated in FIGS. 12A-12C depict the stages of the quick math exercise as presented to the user through the mobile device.

As illustrated in FIG. 12A, the graphical user interface 1202 presented on the display of the mobile device 102 provides the initial interface for the quick math exercise. The interface displays an exercise indicator 1204 identifying the current exercise within a sequence of exercises, such as “Exercise 1/5.” An instructional prompt 1206 instructs the user to calculate the result of the displayed mathematical expression. A timer indicator 1208 visually represents the time remaining for the user to complete the calculation. The interface further displays a mathematical expression 1210, such as “(3+2)*4−6 =?”. A numeric input field 1212 is provided to allow the user to enter the calculated result. A numeric keypad 1214 rendered on the touchscreen allows the user to input the answer using the touchscreen interface of the mobile device.

During this interaction, the foreground sensors required to administer the test include the touchscreen capacitive digitizer of the mobile device 102, which detects numeric keypad selections and records the spatial coordinates associated with each touch event. The system clock functions as a timer that measures the duration between presentation of the mathematical expression 1210 and submission of the user's answer, thereby enabling the system to determine the total response time required for the user to complete the calculation task.

As illustrated in FIG. 12B, once the user submits the response, the graphical interface transitions to a results screen displaying the user's exercise score 1220 and feedback text 1222 indicating whether the answer was correct. The interface may also display optional feedback controls allowing the user to indicate whether the feedback was helpful. A continuation control 1224 allows the user to proceed to the next calculation exercise within the sequence.

As illustrated in FIG. 12C, the system presents a subsequent arithmetic exercise in which the graphical interface again displays a mathematical expression 1230 requiring the user to compute a result, such as “8 * (3+2)=?”. The user enters the calculated answer into the input field 1212 using the numeric keypad 1214 while the mobile device records the associated interaction data and timing characteristics.

While the user is actively interacting with the device to enter the mathematical result, the mobile device 102 simultaneously captures background biometric sensor data from sensors that are not required to administer the arithmetic task but provide additional physiological and behavioral measurements. For example, motion sensors of the mobile device, including a multi-axis accelerometer and gyroscope, may capture micro-movements of the device while the user holds or interacts with the phone. These signals may be analyzed to detect tremor amplitude, tremor frequency characteristics, and device sway patterns during interaction. Additionally, touchscreen pressure sensing capabilities of the device may capture touch pressure variations and subtle finger stability characteristics during numeric keypad interactions. Because these signals are not required to administer the arithmetic exercise itself, they are categorized as background sensor data captured concurrently with the foreground interaction data.

After the user completes the quick math exercise sequence, the mobile device 102 aggregates the captured foreground interaction data and the concurrently captured background biometric sensor data and transmits this information through network 104 to the remote server 10. The server 10 executes analytical algorithms, including artificial intelligence and machine learning models, to evaluate the received data. The server may derive cognitive performance metrics including calculation accuracy, response latency distributions, cognitive processing speed, and numerical reasoning patterns across the presented exercises. Concurrently, background sensor data may be analyzed to derive neurological biomarkers including tremor characteristics derived from accelerometer signals and fine motor interaction patterns derived from touchscreen pressure and numeric entry timing characteristics.

After the server 10 completes its analysis, the calculated arithmetic performance score and associated feedback are transmitted back via network 104 to the mobile device 102. The mobile device updates the graphical user interface 1202 to display the score to the user. The analyzed results are also stored within the user's profile on the server and made accessible through the clinician portal 12. Within the clinician portal 12, the clinician 14 may review longitudinal performance metrics including arithmetic accuracy, response timing distributions, and associated motor interaction characteristics across repeated testing sessions. This information enables the clinician 14 to monitor cognitive health trends and detect early neurological impairments affecting numerical reasoning, executive function, or cognitive processing speed.

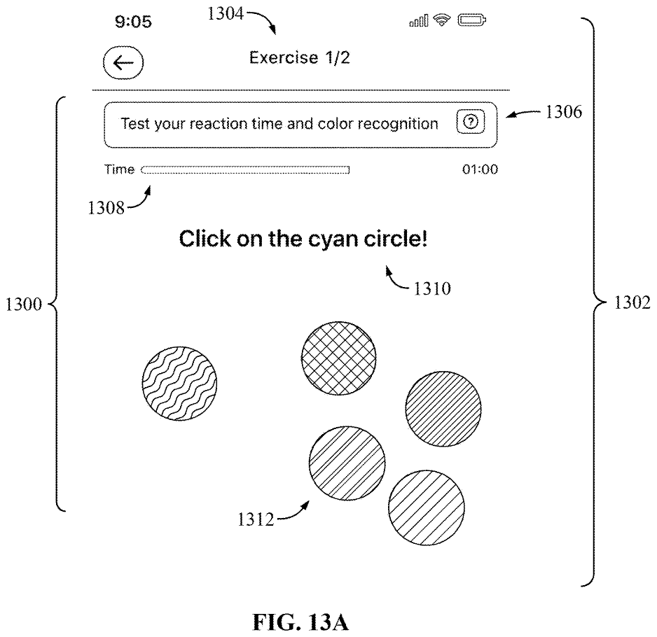

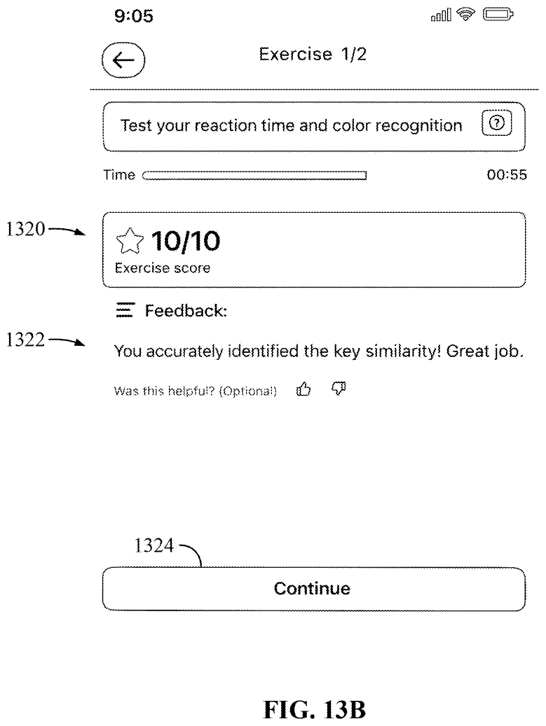

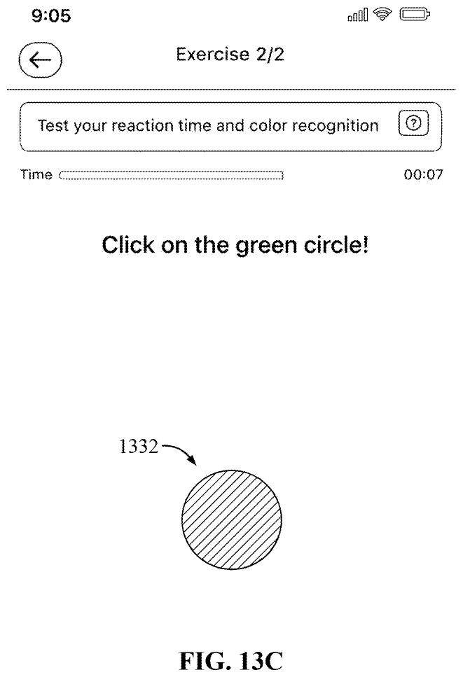

Referring now to FIGS. 13A-13C, the system 100 executes a Reaction Time and Color Recognition Cognitive Test module 1300 on the mobile device 102. The Reaction Time and Color Recognition Cognitive Test module 1300 evaluates reaction speed, visual processing, attention, and cognitive decision-making by requiring the user 16a, 16b to rapidly identify and select a visual stimulus that satisfies a specified condition. This exercise represents a digitized implementation of traditional reaction-time and stimulus discrimination tests commonly used in neurological and neuropsychological examinations to assess processing speed, visual attention, and stimulus-response coordination. The graphical interfaces illustrated in FIGS. 13A-13C depict the stages of the reaction-time testing module as presented to the user through the mobile device.

As illustrated in FIG. 13A, the graphical user interface 1302 presented on the display of the mobile device 102 provides the initial interface for the reaction-time exercise. The interface displays an exercise indicator 1304 identifying the current exercise within a sequence, such as “Exercise 1/2.” An instructional prompt 1306 informs the user that the exercise tests reaction time and color recognition. A timer indicator 1308 displays the remaining time for the exercise session. The interface further presents a stimulus instruction 1310 directing the user to select a specific colored object, for example “Click on the cyan circle.” A plurality of selectable circular objects 1312 are displayed on the screen, each representing potential visual targets. The user must visually identify the circle matching the instructed color and select it as quickly as possible.

During this interaction, the foreground sensors required to administer the test include the touchscreen capacitive digitizer of the mobile device 102, which detects touch selections and records the spatial coordinates associated with the selected object. The system clock functions as a timer that measures the duration between presentation of the visual stimulus and the user's selection of a target object, thereby enabling the system to determine stimulus-to-response reaction time for the exercise. These timestamps enable the system to derive reaction-time metrics including stimulus-to-response latency, selection accuracy, and decision hesitation intervals.

As illustrated in FIG. 13B, once the user successfully selects the correct visual target, the graphical interface transitions to a results screen displaying the user's exercise score 1320 and feedback text 1322 acknowledging the correct selection. The interface may also provide optional feedback controls allowing the user to indicate whether the feedback was helpful. A continuation control 1324 allows the user to proceed to the next reaction-time exercise within the sequence.

As illustrated in FIG. 13C, the system presents an additional reaction-time exercise in which the graphical interface again displays a stimulus instruction 1330 directing the user to select a circle of a specified color, for example “Click on the green circle.” A new set of circular visual stimuli 1332 is displayed on the screen, and the user must again identify and select the correct target as quickly as possible while the mobile device records the associated interaction data and timing characteristics.

While the user is actively interacting with the device to select the visual targets, the mobile device 102 simultaneously captures background biometric sensor data from sensors that are not required to administer the reaction-time task but provide additional physiological and behavioral measurements. For example, motion sensors of the mobile device, including a multi-axis accelerometer and gyroscope, may capture micro-movements of the device while the user holds or interacts with the phone. These signals may be analyzed to detect tremor amplitude, tremor frequency characteristics, and device sway patterns during interaction. Additionally, the touchscreen digitizer may capture touch pressure variations and subtle finger positioning characteristics during target selection. Because these signals are not required to administer the reaction-time exercise itself, they are categorized as background sensor data captured concurrently with the foreground interaction data.

After the user completes the reaction-time exercise sequence, the mobile device 102 aggregates the captured foreground interaction data and the concurrently captured background biometric sensor data and transmits this information through network 104 to the remote server 10. The server 10 executes analytical algorithms, including artificial intelligence and machine learning models, to evaluate the received data. The server may derive cognitive performance metrics including reaction time latency distributions, stimulus recognition accuracy, target selection precision, and cognitive processing speed. Concurrently, background sensor data may be analyzed to derive neurological biomarkers including tremor characteristics derived from accelerometer signals and fine motor interaction patterns derived from touchscreen pressure and touch trajectory characteristics.

After the server 10 completes its analysis, the calculated reaction-time performance score and associated feedback are transmitted back via network 104 to the mobile device 102. The mobile device updates the graphical user interface 1302 to display the score to the user. The analyzed results are also stored within the user's profile on the server and made accessible through the clinician portal 12. Within the clinician portal 12, the clinician 14 may review longitudinal performance metrics including reaction-time distributions, visual discrimination accuracy, response timing characteristics, and associated motor interaction characteristics across repeated testing sessions. This information enables the clinician 14 to monitor cognitive health trends and detect early neurological impairments affecting visual processing speed, attention, or sensorimotor coordination.

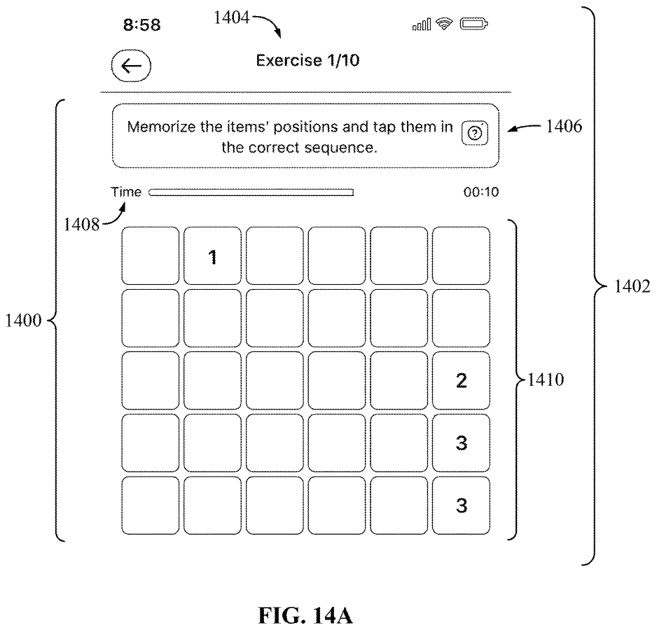

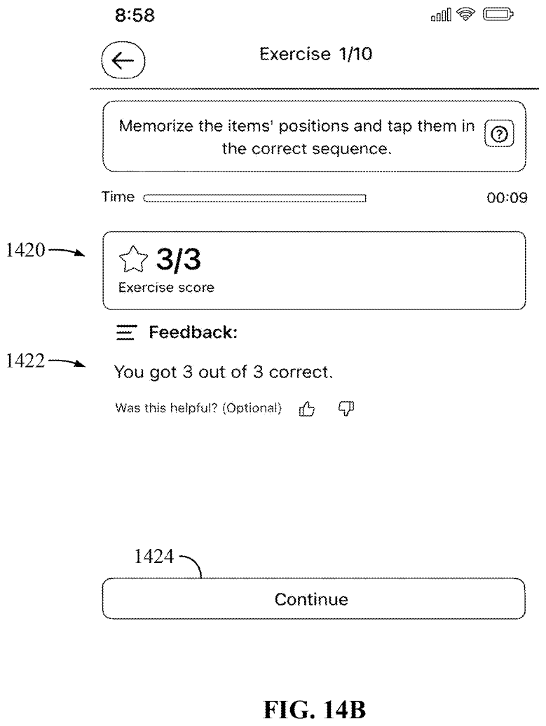

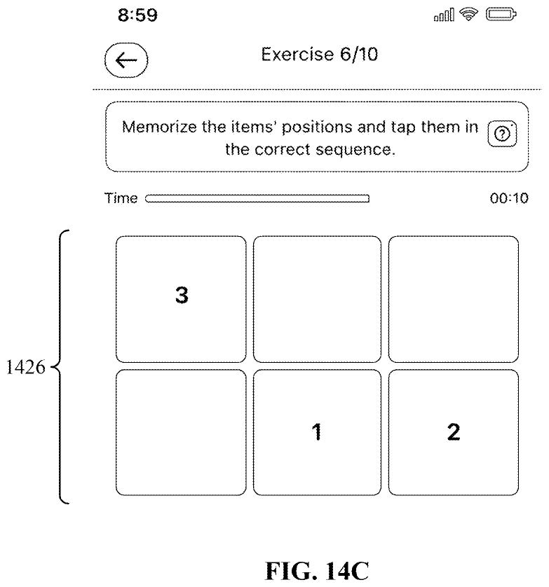

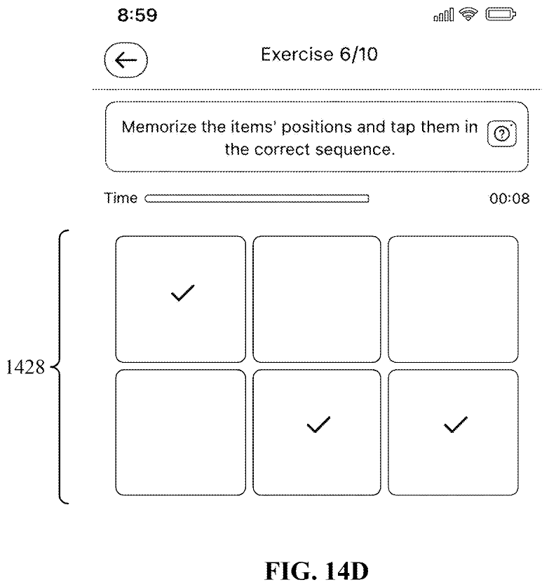

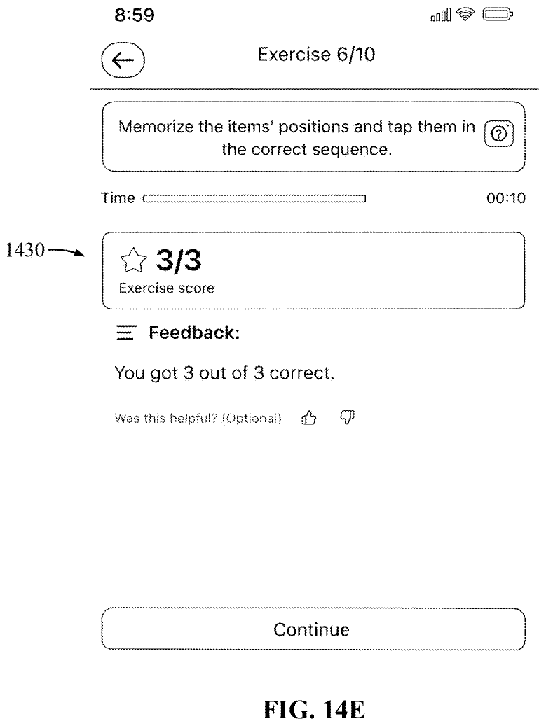

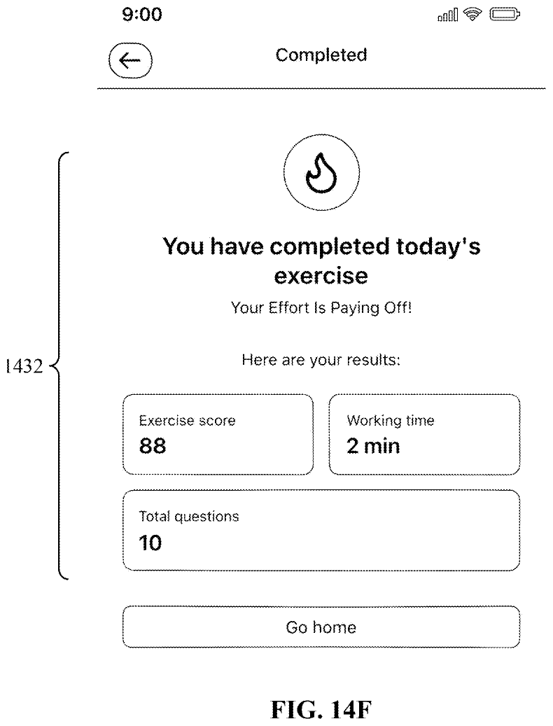

Referring now to FIGS. 14A-14F, the system 100 executes a Sequence Memory Cognitive Test module 1400 on the mobile device 102. The Sequence Memory Cognitive Test module 1400 evaluates short-term memory, spatial memory, attention, and sequential recall by requiring the user 16a, 16b to observe the positions of items displayed on a grid and then reproduce the order in which those items appear. This exercise represents a digitized implementation of traditional sequence memory and spatial recall tests commonly used in neurological and neuropsychological examinations to evaluate working memory capacity, attention, and executive sequencing ability. The graphical interfaces illustrated in FIGS. 14A-14F depict the stages of the sequence memory exercise as presented to the user through the mobile device.

As illustrated in FIG. 14A, the graphical user interface 1402 presented on the display of the mobile device 102 provides the initial interface for the sequence memory exercise. The interface displays an exercise indicator 1404 identifying the current exercise within a sequence of exercises, such as “Exercise 1/10.” An instructional prompt 1406 instructs the user to memorize the positions of items appearing on a grid and to subsequently tap the items in the correct sequence. A timer indicator 1408 displays the time remaining for the memorization and response interval. The interface further presents a grid 1410 comprising a plurality of selectable cells arranged in rows and columns. Certain cells temporarily display numbered indicators, such as “1,” “2,” and “3,” which represent the order in which the user must later reproduce the sequence.

During the memorization phase, the numbered indicators appear within selected grid cells for a brief period of time before being removed. After the indicators disappear, the user must recall the positions and tap the corresponding grid cells in the correct sequential order. The foreground sensors required to administer the test include the touchscreen capacitive digitizer of the mobile device 102, which detects the user's touch selections on the grid cells and records the spatial coordinates associated with each tap. The system clock functions as a timer that measures the duration between presentation of the memorization sequence and the user's completion of the recalled sequence, thereby enabling the system to determine total recall time for the exercise.

As illustrated in FIG. 14B, after the user completes the sequence selection for the first exercise, the graphical interface displays an exercise score 1420 and feedback text 1422 indicating the number of correctly recalled positions. The interface may also provide optional feedback controls allowing the user to indicate whether the feedback was helpful. A continuation button 1424 allows the user to proceed to the next sequence memory exercise.

As illustrated in FIGS. 14C and 14D, subsequent exercises present new grid configurations 1426 in which numbered indicators appear in different positions across the grid. The user again observes the displayed sequence and then taps the grid cells in the order corresponding to the memorized sequence. The interface may visually confirm correct selections 1428 by displaying check marks or similar indicators within the tapped cells. During these interactions, the mobile device captures foreground interaction data including tap coordinates and sequence order selections corresponding to the user's recalled pattern.

While the user performs the sequence recall task, the mobile device 102 simultaneously captures background biometric sensor data from sensors that are not required to administer the sequence memory task but provide additional physiological and behavioral measurements. For example, motion sensors of the mobile device, including a multi-axis accelerometer and gyroscope, may capture micro-movements of the device while the user holds or interacts with the phone. These signals may be analyzed to detect tremor amplitude, tremor frequency characteristics, and device sway patterns during interaction. Additionally, touchscreen pressure sensing capabilities of the device may capture touch pressure variations and subtle finger stability characteristics during grid cell selections. Because these signals are not required to administer the sequence recall exercise itself, they are categorized as background sensor data captured concurrently with the foreground interaction data.

As illustrated in FIG. 14E, after the user completes a later exercise within the sequence, the interface again displays an exercise score 1430 and feedback text acknowledging the number of correctly recalled positions. A continuation control allows the user to proceed to additional exercises within the sequence.

As illustrated in FIG. 14F, the mobile device displays a completion interface 1432 indicating that the user has completed the sequence memory exercise session. The interface may display summary information including the exercise score, total working time, and the total number of questions completed during the session.

At the conclusion of the exercise session, the mobile device 102 aggregates the captured foreground interaction data and the concurrently captured background biometric sensor data and transmits this information through network 104 to the remote server 10. The server 10 executes analytical algorithms, including artificial intelligence and machine learning models, to evaluate the received data. The server may derive cognitive performance metrics including sequence recall accuracy, spatial memory performance, recall latency distributions, and sequential decision patterns across the exercises. Concurrently, background sensor data may be analyzed to derive neurological biomarkers including tremor characteristics derived from accelerometer signals and fine motor interaction patterns derived from touchscreen pressure and touch trajectory characteristics.

After the server 10 completes its analysis, the calculated sequence memory performance score and associated feedback are transmitted back via network 104 to the mobile device 102. The mobile device updates the graphical user interface 1402 to display the score to the user. The analyzed results are also stored within the user's profile on the server and made accessible through the clinician portal 12. Within the clinician portal 12, the clinician 14 may review longitudinal performance metrics including spatial recall accuracy, sequence memory capacity, response timing distributions, and associated motor interaction characteristics across repeated testing sessions. This information enables the clinician 14 to monitor cognitive health trends and detect early neurological impairments affecting working memory, spatial recall, or executive sequencing ability.

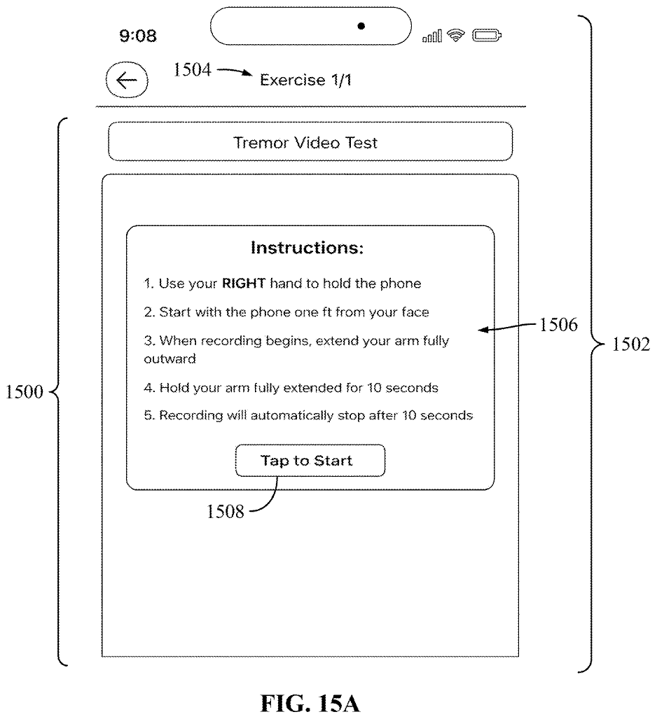

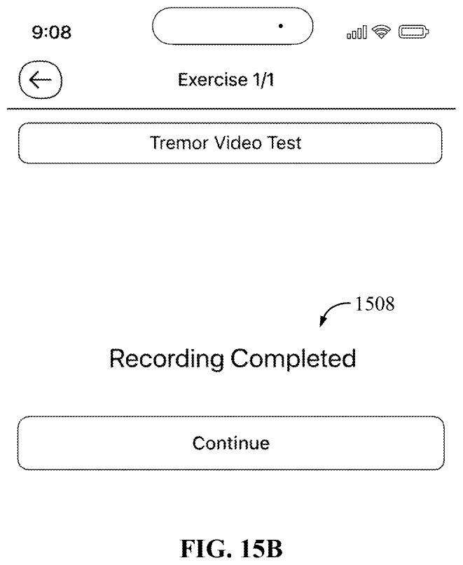

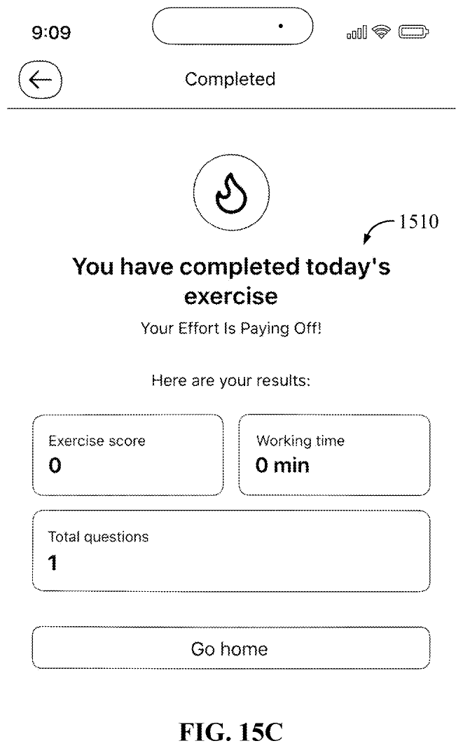

Referring now to FIGS. 15A-15C, the system 100 executes a Tremor Assessment module 1500 on the mobile device 102. The Tremor Assessment module 1500 evaluates upper-limb tremor characteristics, motor stability, and involuntary oscillatory movement by capturing motion and video data while the user performs a controlled arm-extension task. This exercise represents a digitized implementation of clinical tremor examinations commonly performed during neurological evaluations for conditions such as Parkinson's disease, essential tremor, and other motor disorders. The graphical interfaces illustrated in FIGS. 15A-15C depict the stages of the tremor assessment module as presented to the user through the mobile device.

As illustrated in FIG. 15A, the graphical user interface 1502 presented on the display of the mobile device 102 provides the introductory screen for the tremor video test. The interface displays an exercise indicator 1504 identifying the current exercise, such as “Exercise 1/1.” An instructional panel 1506 presents step-by-step instructions directing the user to hold the mobile device with the right hand, begin with the device approximately one foot from the face, extend the arm fully outward when recording begins, and maintain the arm in the extended position for a predefined duration, such as ten seconds. The interface further includes a start control 1508 labeled “Tap to Start,” which allows the user to initiate the recording session.

When the user activates the start button 1508, the mobile device 102 begins a recording interval during which the device captures motion and video data associated with the user's arm position and movement stability. During this phase, the foreground sensors required to administer the test include the device camera, which records video of the user's arm and device orientation, and the multi-axis accelerometer and gyroscope, which capture motion signals representing tremor amplitude, tremor frequency, and stability of the arm while extended. The system clock functions as a timer that measures the duration of the recording interval from initiation of the arm-extension task until completion of the recording session, thereby enabling the system to determine the measurement period during which tremor characteristics are captured.

As illustrated in FIG. 15B, once the recording interval concludes, the graphical interface displays a status message 1508 indicating that the recording has been completed. A continuation control allows the user to proceed to the next stage of the exercise workflow.

While the tremor assessment is being performed, the mobile device 102 may simultaneously capture background biometric sensor data from sensors that are not required to administer the tremor measurement itself but provide additional behavioral or environmental context. For example, touchscreen pressure sensing capabilities of the device may capture incidental touch pressure signals while the user holds the device, and the device microphone may capture ambient environmental audio during the recording interval. Because these signals are not required to administer the tremor measurement itself, they are categorized as background sensor data captured concurrently with the foreground motion and video data.

At the conclusion of the recording interval, the mobile device 102 aggregates the captured foreground motion and video data and the concurrently captured background biometric sensor data and transmits this information through network 104 to the remote server 10. The server 10 executes analytical algorithms, including artificial intelligence and machine learning models, to evaluate the received data. The server may derive neurological performance metrics including tremor amplitude, dominant tremor frequency bands, tremor variability, and arm stability characteristics associated with the user's extended-arm posture. In certain embodiments, computer vision algorithms may analyze the captured video stream to identify visual indicators of tremor movement or arm drift during the recording interval.

After the server 10 completes its analysis, the derived tremor assessment metrics may be transmitted back via network 104 to the mobile device 102. The mobile device may update the graphical user interface to indicate a message 1510 that the exercise has been completed, as illustrated in FIG. 15C, which displays a completion interface summarizing the exercise session. The analyzed results are also stored within the user's profile on the server and made accessible through the clinician portal 12. Within the clinician portal 12, the clinician 14 may review longitudinal tremor measurements and stability indicators collected across repeated testing sessions. These measurements enable the clinician 14 to monitor neurological health trends and detect early motor impairments associated with neurodegenerative or movement disorders.

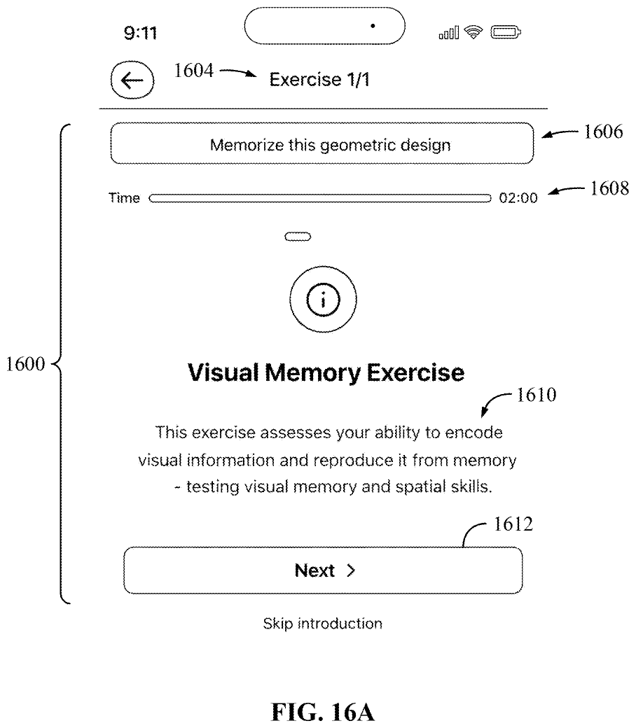

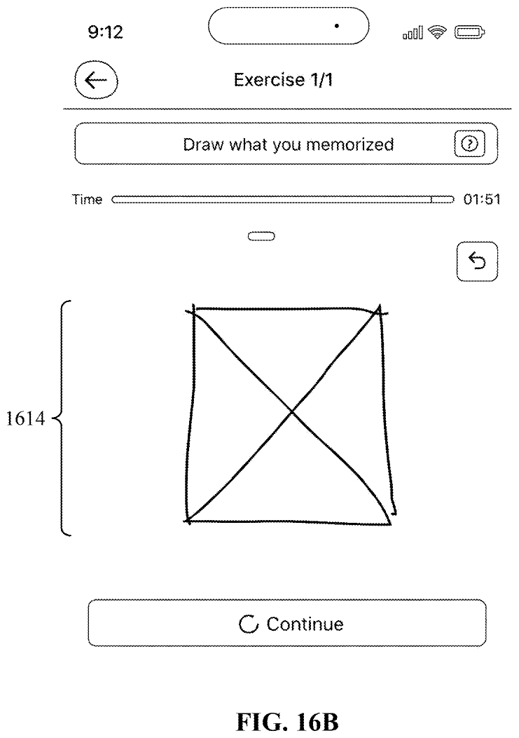

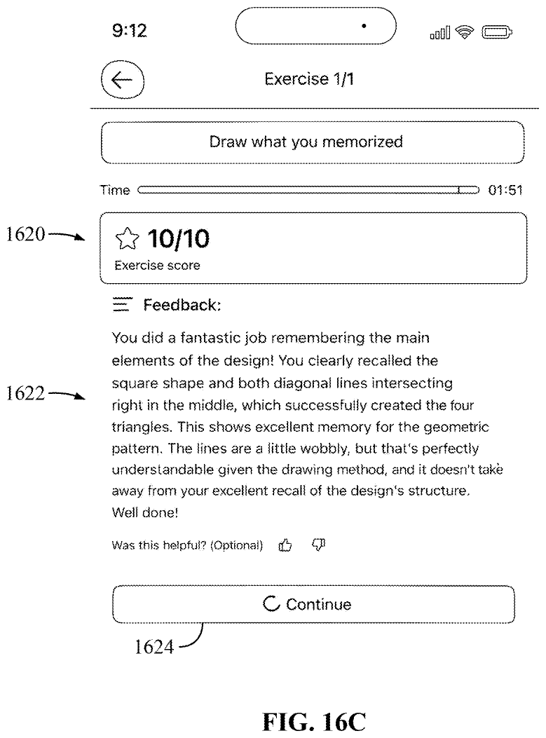

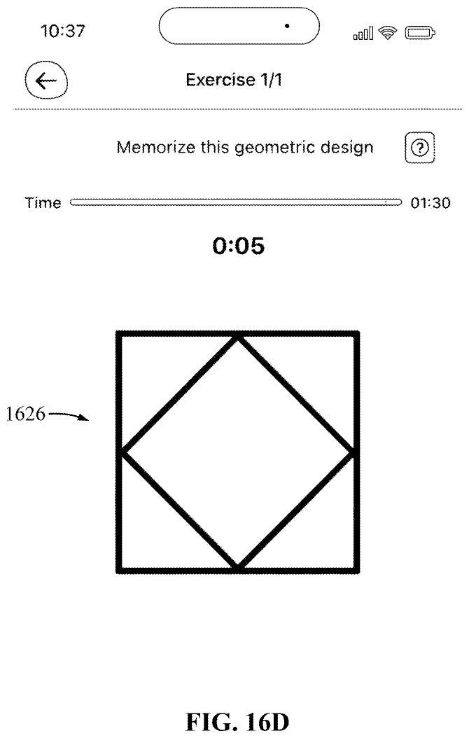

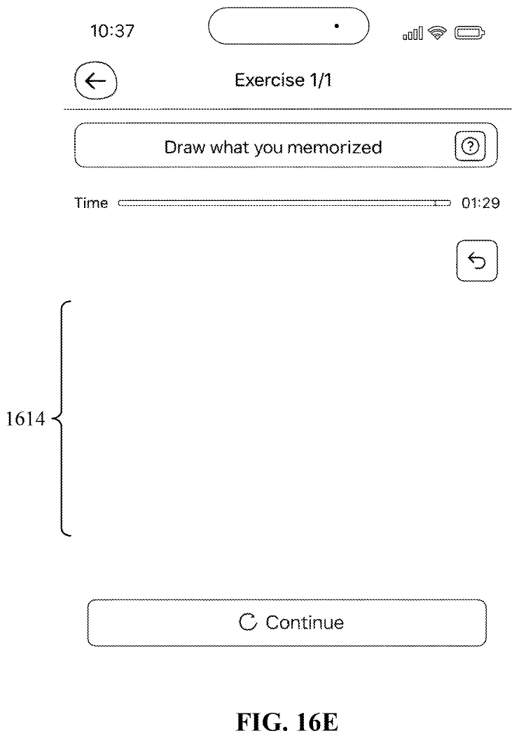

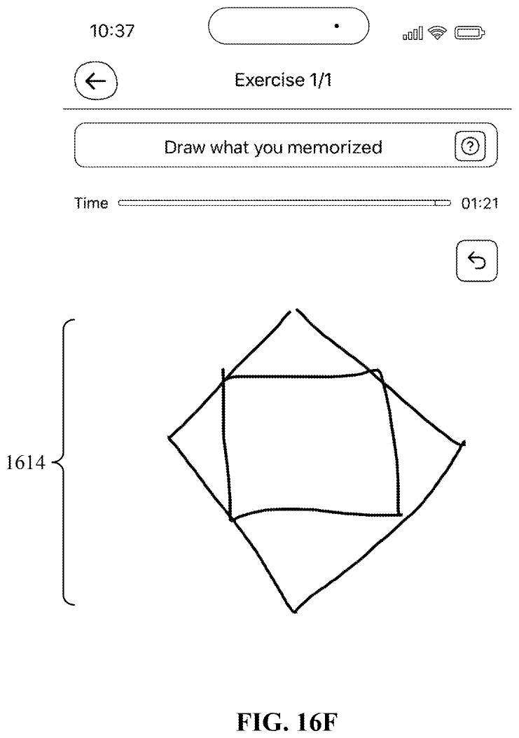

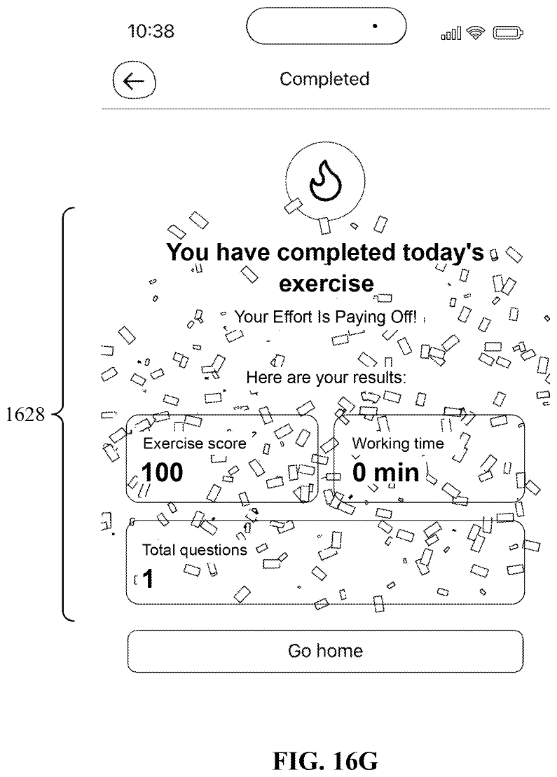

Referring now to FIGS. 16A-16G, the system 100 executes a Visual Reproduction Cognitive Test module 1600 on the mobile device 102. The Visual Reproduction Cognitive Test module 1600 evaluates visual memory, spatial reasoning, and visuomotor coordination by requiring the user 16a, 16b to observe a geometric design, memorize the configuration of the design, and subsequently reproduce the design from memory using the touchscreen interface of the mobile device. This exercise represents a digitized implementation of traditional visual memory reproduction tests commonly used in neurological and neuropsychological examinations to evaluate visual encoding ability, spatial memory retention, and visuospatial processing. The graphical interfaces illustrated in FIGS. 16A-16G depict the stages of the visual reproduction exercise as presented to the user through the mobile device.

As illustrated in FIG. 16A, the graphical user interface 1602 presented on the display of the mobile device 102 provides an introductory interface for the visual memory exercise. The interface displays an exercise indicator 1604 identifying the current exercise within the session, such as “Exercise 1/1.” An instructional prompt 1606 instructs the user to memorize a geometric design presented on the screen. A timer indicator 1608 displays the time allocated for completing the exercise, such as approximately two minutes. An informational description 1610 explains that the visual memory exercise evaluates the user's ability to encode visual information and reproduce it from memory, thereby testing visual memory and spatial skills. A continuation control 1612 allows the user to proceed to the memorization phase of the exercise.

As illustrated in FIG. 16B, once the memorization phase begins, the graphical interface presents a drawing workspace 1614 in which the user is instructed to “Draw what you memorized.” During this stage, the mobile device 102 activates the foreground sensors required to administer the test. These sensors include the touchscreen capacitive digitizer, which captures the X-Y coordinate trajectories of the user's finger as the user draws lines representing the memorized geometric design. The touchscreen digitizer records the spatial trajectory of the user's drawing strokes representing the reproduced geometric design. The system clock functions as a timer that measures the duration of the drawing task from presentation of the reproduction workspace until completion of the user's reproduced design, thereby enabling the system to determine the total working time required for the visual reproduction task.

As illustrated in FIG. 16C, after the user completes the reproduction of the design, the graphical interface presents an exercise score 1620 and feedback text 1622 evaluating the accuracy of the reproduced design. The feedback may describe whether the user successfully recalled key structural elements of the geometric pattern, such as the square outline and intersecting diagonal lines forming triangular segments. A continuation control 1624 allows the user to proceed through the exercise workflow.

As illustrated in FIG. 16D, the system may present an additional geometric design 1626 during the memorization phase of the exercise. In this example, the interface displays a rectangular frame containing a diamond-shaped interior figure formed by intersecting diagonal segments. The user is provided a brief viewing period during which the design must be memorized before it disappears.

As illustrated in FIG. 16E, once the memorization interval expires, the graphical interface again presents the drawing workspace 1614 with the instruction to reproduce the memorized geometric design from memory. The user draws the design on the touchscreen interface using finger gestures while the mobile device captures the associated drawing data.

As illustrated in FIG. 16F, the user's reproduction of the memorized geometric design appears within the drawing workspace 1614 as the user sketches the lines corresponding to the recalled shapes. During this drawing interaction, the mobile device continuously captures foreground interaction data including stroke trajectories corresponding to the user's reproduced drawing, and the spatial relationships between drawn line segments.

While the user is actively engaged in reproducing the geometric design, the mobile device 102 simultaneously captures background biometric sensor data from sensors that are not required to administer the drawing task but provide additional physiological and behavioral information. For example, the multi-axis accelerometer and gyroscope of the mobile device may capture micro-movements of the device while the user holds or interacts with the phone. These motion signals may be analyzed to detect tremor amplitude, tremor frequency characteristics, and device sway patterns during drawing interactions. Additionally, touchscreen pressure sensing capabilities of the device may capture variations in touch pressure and stroke stability, which may reveal subtle motor control characteristics. Because these signals are not required to administer the visual memory exercise itself, they are categorized as background sensor data captured concurrently with the foreground interaction data.

As illustrated in FIG. 16G, after completion of the exercise session, the graphical interface presents a completion screen 1628 indicating that the user has finished the day's exercise. The interface may display summary metrics including an exercise score, working time, and the number of questions completed during the session.

At the conclusion of the visual reproduction exercise, the mobile device 102 aggregates the captured foreground drawing interaction data and the concurrently captured background biometric sensor data and transmits this information through network 104 to the remote server 10. The server 10 executes analytical algorithms, including artificial intelligence and machine learning models, to evaluate the received data. The server may derive cognitive performance metrics including structural accuracy of the reproduced design, spatial alignment of drawn elements, stroke sequencing patterns, and drawing completion time. Concurrently, background sensor data may be analyzed to derive neurological biomarkers including tremor characteristics derived from accelerometer signals and fine motor control metrics derived from drawing stroke smoothness and pressure variation.

After the server 10 completes its analysis, the calculated visual memory performance score and associated feedback are transmitted back via network 104 to the mobile device 102. The mobile device updates the graphical user interface 1602 to display the score and feedback to the user. The analyzed results are also stored within the user's profile on the server and made accessible through the clinician portal 12. Within the clinician portal 12, the clinician 14 may review longitudinal performance metrics including visual memory accuracy, spatial reproduction fidelity, drawing stroke characteristics, and associated motor interaction patterns across repeated testing sessions. This information enables the clinician 14 to monitor cognitive health trends and detect early neurological impairments affecting visual memory, visuospatial processing, or fine motor coordination.

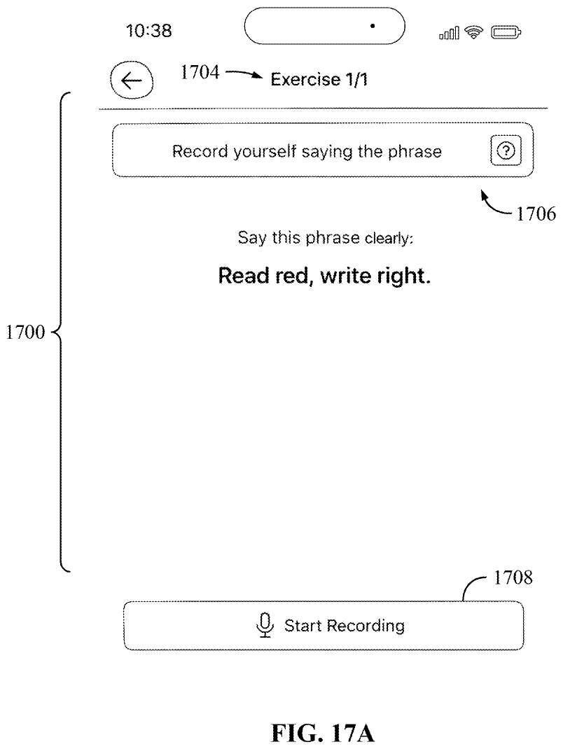

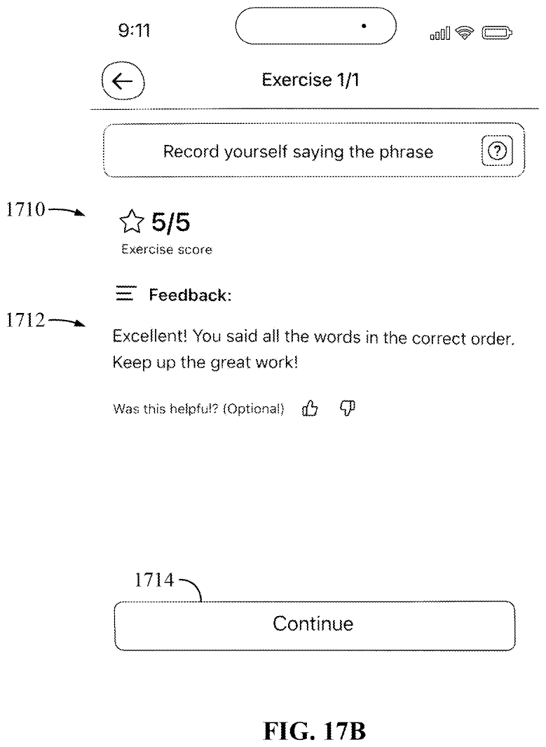

Referring now to FIGS. 17A-17B, the system 100 executes a Voice Recording and Speech Assessment module 1700 on the mobile device 102. The Voice Recording and Speech Assessment module 1700 evaluates speech articulation, verbal fluency, phonetic accuracy, and language processing by requiring the user 16a, 16b to record themselves speaking a specified phrase presented on the screen. This exercise represents a digitized implementation of traditional speech and language tests commonly used in neurological and neuropsychological examinations to evaluate speech clarity, cognitive language processing, and articulation control. The graphical interfaces illustrated in FIGS. 17A-17B depict the stages of the voice recording exercise as presented to the user through the mobile device.

As illustrated in FIG. 17A, the graphical user interface 1702 presented on the display of the mobile device 102 provides the interface for the voice recording exercise. The interface displays an exercise indicator 1704 identifying the current exercise within the session, such as “Exercise 1/1.” An instructional prompt 1706 directs the user to record themselves speaking a displayed phrase. In the illustrated example, the user is instructed to clearly say the phrase “Read red, write right.” The interface further includes a recording control 1708 labeled “Start Recording,” which the user selects to begin the audio capture session.

When the user activates (i.e., push the button) the start recording control 1708, the mobile device 102 begins capturing the user's spoken audio. During this phase, the foreground sensors required to administer the test include the microphone of the mobile device 102, which records the user's voice as an audio signal. The system clock functions as a timer that measures the duration of the audio recording interval from initiation of the recording control 1708 until completion of the spoken response, thereby enabling the system to determine speech duration and response latency. The recorded audio stream constitutes the primary foreground interaction data used to evaluate the user's verbal response.

While the user performs the speech recording task, the mobile device 102 may simultaneously capture background biometric sensor data from sensors that are not required to administer the speech recording exercise but provide additional behavioral and physiological context. For example, motion sensors of the mobile device, including a multi-axis accelerometer and gyroscope, may capture subtle device movements while the user holds the phone during speech. These motion signals may be analyzed to detect tremor amplitude or device sway patterns associated with motor control characteristics. Additionally, touchscreen pressure sensing capabilities may capture incidental touch pressure or grip interaction patterns while the device is held. Because these sensor streams are not required to administer the speech recording exercise itself, they are categorized as background sensor data captured concurrently with the foreground audio data.

After the user completes the speech recording, the mobile device 102 aggregates the captured foreground audio recording data and the concurrently captured background biometric sensor data and transmits this information through network 104 to the remote server 10. The server 10 executes analytical algorithms, including artificial intelligence and machine learning models, to evaluate the received audio data. Speech processing algorithms may analyze the audio recording to evaluate pronunciation accuracy, articulation clarity, phoneme timing, and correct ordering of the spoken words within the phrase. The system may also derive speech-related biomarkers including speech rate, vocal tremor characteristics, articulation stability, and pauses between spoken words.

After the server 10 completes its analysis, the derived speech performance score and associated feedback are transmitted back via network 104 to the mobile device 102. As illustrated in FIG. 17B, the graphical interface displays the calculated exercise score 1710 and feedback text 1712 indicating that the user correctly pronounced the phrase in the proper order. A continuation control 1714 allows the user to proceed within the application workflow.

The analyzed results are also stored within the user's profile on the server and made accessible through the clinician portal 12. Within the clinician portal 12, the clinician 14 may review longitudinal speech performance metrics including pronunciation accuracy, speech rate, articulation clarity, and vocal stability across repeated testing sessions. This information enables the clinician 14 to monitor neurological health trends and detect early impairments affecting speech production, language processing, or motor control associated with neurological disorders or cognitive decline.

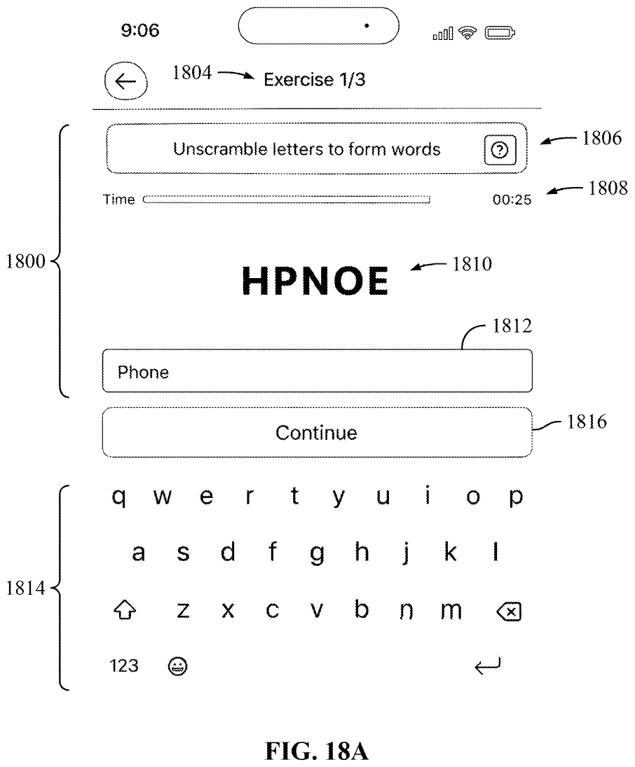

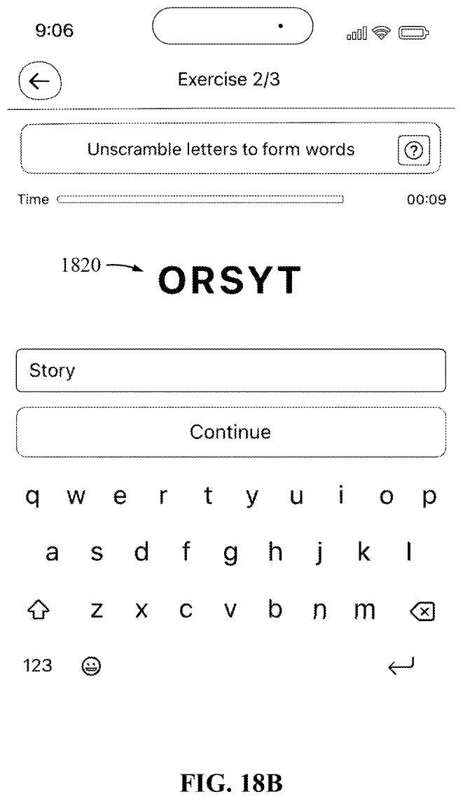

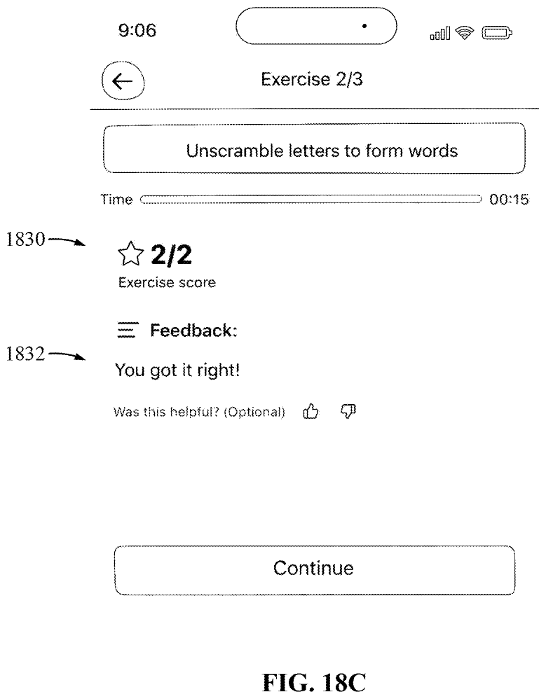

Referring now to FIGS. 18A-18C, the system 100 executes a Word Scramble Cognitive Test module 1800 on the mobile device 102. The Word Scramble Cognitive Test module 1800 evaluates language processing, lexical retrieval, spelling ability, and cognitive flexibility by requiring the user 16a, 16b to mentally reorganize a set of scrambled letters into a correctly spelled word. This exercise represents a digitized implementation of traditional word reconstruction and lexical processing tests used in neurological and neuropsychological examinations to evaluate language comprehension, vocabulary access, and executive cognitive processing. The graphical interfaces illustrated in FIGS. 18A-18C depict the stages of the word scramble exercise as presented to the user through the mobile device.

As illustrated in FIG. 18A, the graphical user interface 1802 presented on the display of the mobile device 102 provides the interactive interface for the word scramble exercise. The interface displays an exercise indicator 1804 identifying the current exercise within the session, such as “Exercise 1/3.” An instructional prompt 1806 directs the user to unscramble letters to form a valid word. A timer indicator 1808 displays the remaining time allocated to solve the puzzle. In the illustrated example, a scrambled letter sequence 1810 such as “HPNOE” is displayed to the user. The user must mentally rearrange the letters to form the correct word. An answer input field 1812 allows the user to enter the reconstructed word, and a touchscreen keyboard 1814 is presented to allow the user to type the response. A continuation control 1816 allows the user to submit the entered word and proceed to the next puzzle.

During this interaction, the foreground sensors required to administer the test include the touchscreen capacitive digitizer of the mobile device 102, which detects keystroke inputs on the touchscreen keyboard and records the spatial coordinates associated with each typed character. The system clock functions as a timer that measures the duration between presentation of the scrambled letter sequence 1810 and submission of the user's reconstructed word, thereby enabling the system to determine the total problem-solving time for the exercise. These timestamps enable the system to derive response latency, typing speed, inter-keystroke timing intervals, and total problem-solving time for the puzzle.

As illustrated in FIG. 18B, the system presents a subsequent word scramble puzzle during the exercise session. In this example, the scrambled letter sequence 1820 “ORSYT” is displayed, and the user reconstructs the correct word by entering the response “Story” into the answer input field using the touchscreen keyboard. As with the previous puzzle, the mobile device records the interaction data associated with the user's typed response and submission of the reconstructed word.

While the user performs the word reconstruction task, the mobile device 102 simultaneously captures background biometric sensor data from sensors that are not required to administer the word scramble exercise but provide additional behavioral and physiological context. For example, motion sensors of the mobile device, including a multi-axis accelerometer and gyroscope, may capture micro-movements of the device while the user holds or interacts with the phone. These signals may be analyzed to detect tremor amplitude, tremor frequency characteristics, and device sway patterns during typing interactions. Additionally, touchscreen pressure sensing capabilities of the device may capture touch pressure characteristics and subtle finger positioning adjustments during keyboard entry. Because these sensor streams are not required to administer the word scramble exercise itself, they are categorized as background sensor data captured concurrently with the foreground interaction data.

As illustrated in FIG. 18C, after the user completes the word scramble exercise, the graphical interface displays an exercise score 1830 and feedback text 1832 indicating that the user correctly solved the puzzle. A continuation control 1834 allows the user to proceed to the next stage of the application workflow.

At the conclusion of the exercise session, the mobile device 102 aggregates the captured foreground interaction data and the concurrently captured background biometric sensor data and transmits this information through network 104 to the remote server 10. The server 10 executes analytical algorithms, including artificial intelligence and machine learning models, to evaluate the received data. The server may derive cognitive performance metrics including word reconstruction accuracy, lexical processing speed, response latency distributions, and error correction patterns during word entry. Concurrently, background sensor data may be analyzed to derive neurological biomarkers including tremor characteristics derived from accelerometer signals and fine motor typing patterns derived from touch pressure and keystroke timing characteristics.

After the server 10 completes its analysis, the calculated word scramble performance score and associated feedback are transmitted back via network 104 to the mobile device 102. The mobile device updates the graphical user interface 1802 to display the score to the user. The analyzed results are also stored within the user's profile on the server and made accessible through the clinician portal 12. Within the clinician portal 12, the clinician 14 may review longitudinal performance metrics including lexical reconstruction accuracy, response timing distributions, and associated motor interaction characteristics across repeated testing sessions. This information enables the clinician 14 to monitor cognitive health trends and detect early neurological impairments affecting language processing, lexical retrieval, or executive cognitive function.

For each testing module, the mobile device utilizes a defined set of sensors required to administer the test, which are categorized as foreground sensors and produce foreground sensor data used to execute the test. In addition, other sensors of the mobile device that are not required for administration of the test may be activated to collect additional user data and are categorized as background sensors producing background sensor data. The following describes, for each testing module, representative foreground sensors, background sensors, the measurable signals produced by those sensors, the neurological features or biomarkers derived from the signals, and the neurological conditions or diseases that may be evaluated based on those biomarkers.

For each administered cognitive test, including both the Alternating Sequence Test described previously and the Drawing Challenge Test of FIGS. 5A-5F, the data flow architecture remains consistent. The user interacts with the mobile device 102, which captures both foreground performance data and background biometric sensor data. This data is encrypted and transmitted via network 104 to the remote server 10. The server processes the data using AI/ML models to generate macroscopic performance scores and microscopic neurological indicators. The processed results are returned to the mobile device 102 for immediate user feedback and are simultaneously stored within the clinician portal 12 for professional review and longitudinal tracking. Clinician-entered diagnostic flags, including confirmation of neurological disease or confirmation of stable health, are incorporated into the AI training pipeline to continuously refine predictive accuracy.

In certain embodiments, the artificial intelligence and machine learning architecture of the server 10 may analyze foreground sensor data alone, background sensor data alone, or a combined dataset comprising both foreground and background data. The combined analysis enables the system to detect subtle neurological patterns that may not be detectable when evaluating either data stream independently.

Viewed as a whole, the system 100 operates as an integrated predictive neurological monitoring platform comprising the sensor-rich mobile device 102, secure bidirectional network 104 communication, cloud-based server 10 hosting AI-driven analytics, and clinician portal 12 providing oversight and treatment management. The dual-layer architecture—Layer 1 capturing conscious foreground task performance and Layer 2 capturing subconscious background biometric signatures—enables detection of hidden neurological patterns prior to traditional macroscopic score decline. By continuously aggregating data from a plurality of users 16a, 16b and incorporating clinician-confirmed outcome flags, the AI system iteratively improves its ability to identify early disease signatures and early indicators of treatment efficacy. Consequently, the system 100 transforms conventional reactive cognitive testing into a proactive, continuously learning ecosystem to increase predictive diagnosis, dynamic treatment monitoring, and earlier clinical intervention than traditional methods allow.

Utilizing these basic minimum sensors which are included within a category of sensors classified as Foreground Sensors, traditional systems capture foreground interaction data to generate specific macroscopic derived metrics. For the Alternating Sequence Selection test, the foreground task completion score (Layer 1) is based on macroscopic metrics including the total completion time, the number of incorrect selections, the longest correctly maintained alternating sequence, and a binary evaluation of sequence completion versus timeout. For the Drawing Challenge test, the raw foreground image data is traditionally evaluated—often via a Cloud-Vision AI or Large Language Model (LLM)—to extract macroscopic metrics such as drawing completion, overall drawing complexity, drawing sequence organization, and spatial coherence confirming the structural accuracy of the requested elements.

Traditionally, clinicians 14 utilize these macroscopic metrics to diagnose broad cognitive impairments, spatial neglect, or the progression of neurodegenerative diseases. Furthermore, in traditional longitudinal tracking, the traditional marker indicating that a patient 16a, 16b is in neurological decline is a macroscopic “inflection point” or a downward trend in these foreground task scores over time. For example, a clinician 14 reviewing the patient's file may note that the patient 16a, 16b historically completed the alternating sequence with high selection efficiency and drew spatially accurate figures, but over recent months, their completion time has steadily degraded and their drawings have become severely distorted. This macroscopic drop in overall foreground performance is the traditional marker used to confirm that the patient's cognitive state is actively deteriorating.

In addition to the foregoing foreground sensor architecture, the present system 100 also continuously tracks high-fidelity data captured by the background sensors of the mobile device 102. By collecting and analyzing this background sensor data, the system 100 is able to measure and track additional physiological and neurobehavioral conditions that may not be detectable through the macroscopic foreground task score alone. For example, background sensor streams from the multi-axis accelerometer and gyroscope may be processed to extract micro-oscillations, device sway, and tremor signatures, while the operating system event logger and high-fidelity touch digitizer may be processed to quantify sub-perceptual reaction time delays, interaction hesitation, pressure instability, fine motor jitter, and hemispatial interaction asymmetry.

When a disease, impairment, or clinically relevant condition is formally diagnosed by the clinician 14, the clinician 14 records the diagnosis in the patient's record within the clinician portal 12 by entering a supervised clinical flag and associating the flag with a corresponding diagnosis date. Upon entry of this diagnosis flag, the server 10 initiates a retrospective machine learning analysis on that patient's historical data. In particular, the AI/ML architecture of the server 10 performs a lookback analysis to determine whether a recognizable predictive pattern existed in the background sensor data, the foreground sensor data, or a combination thereof, during the days, weeks, or months prior to the diagnosis date, and in certain embodiments prior to any macroscopic decline in the patient's conventional foreground test scores. The retrospective analysis may further include other sensor datasets captured by the mobile device 102, including contextual wellness sensors and derived health metrics, to reduce confounding variables and improve pattern recognition.

Accordingly, the retrospective pattern recognition process described above is not merely a post hoc clinical review tool, but forms part of the continuous operational workflow of the system 100. Specifically, whenever a test is being performed on the mobile device 102, the system 100 is collecting both foreground sensor data and background sensor data, transmitting such data to the server 10, and processing the incoming streams through the AI/ML architecture. During this processing, the server 10 compares the newly captured data to previously learned biometric signatures and predictive patterns derived from prior supervised clinical flags across the patient population. Thus, while the user 16a, 16b experiences the testing modules as simple exercises and receives a macroscopic score, the underlying system 100 is simultaneously executing predictive analytics on the background sensor signals and high-resolution interaction metadata to identify early indicators of neurological decline or treatment response.

Furthermore, the present system 100 is configured to correlate and analyze patterns across a plurality of distinct tests rather than evaluating each test in isolation. In one embodiment, the AI/ML architecture on the server 10 aggregates derived features from multiple modules—including, by way of example, alternating sequence selection performance, drawing-based visuospatial coherence, touch pressure stability, tremor amplitude, hemispatial asymmetry, and other cross-domain features—to identify multi-test signatures that indicate a leading disease, precursor condition, or a disease that commonly occurs before another disease. By analyzing cross-test combinations, temporal relationships, and multi-domain impairment trajectories, the system 100 can determine that certain detectable conditions tend to precede, predict, or co-occur with other conditions, thereby enabling the clinician portal 12 to surface earlier warnings, differentiated risk scoring, and more targeted treatment recommendations than would be possible using any single test score alone.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method for predictive neurological assessment and clinical decision support via mobile device sensor fusion, the method comprising:
rendering a digitized cognitive testing module on a display screen of a mobile computing device;
capturing foreground interaction data via the mobile computing device as a user interacts with the digitized cognitive testing module;
simultaneously capturing background biometric sensor data while the user interacts with the digitized cognitive testing module, wherein capturing the background biometric sensor data comprises:
polling continuous multi-axis motion data utilizing an accelerometer and a gyroscope of the mobile computing device to extract micro-oscillation and device sway metrics;
logging spatial coordinate data and haptic pressure data utilizing a capacitive touch digitizer of the mobile computing device; and
timestamping individual screen interactions utilizing a native operating system event logger of the mobile computing device to extract millisecond-level inter-tap latency and motor planning metrics;
transmitting the foreground interaction data and the background biometric sensor data to a remote server;
calculating a foreground task completion score based on the captured foreground interaction data on the remote server;
processing the background biometric sensor data via a trained machine learning model executing on the remote server to detect a sub-perceptual biometric anomaly, wherein the machine learning model is trained on a historical dataset comprising background biometric sensor data from a plurality of patients correlated with supervised clinical diagnosis flags;
generating a predictive trajectory vector indicating a future onset of a neurological condition based on the detected sub-perceptual biometric anomaly prior to a macroscopic decline in the user's foreground task completion score; and
outputting an alert and a corresponding specific treatment plan recommendation to a secure clinician portal based on the generated predictive trajectory vector.

2. The method of claim 1, wherein training the machine learning model comprises:
receiving a supervised clinical diagnosis flag associated with a specific patient of the plurality of patients, the clinical diagnosis flag establishing a date of formal neurological diagnosis; and
executing a retrospective analysis on the historical dataset of the specific patient to identify an early-warning biometric signature occurring in the background biometric sensor data prior to the date of formal neurological diagnosis.

3. The method of claim 1, wherein the background biometric sensor data is captured continuously by the mobile computing device during non-clinical, gamified application usage independently of the digitized cognitive testing module.

4. The method of claim 1, further comprising a method for predictive treatment efficacy tracking, comprising:
receiving a treatment initiation flag via the clinician portal indicating the recommended specific treatment plan has been prescribed to the user;
continuously receiving subsequent foreground interaction data and subsequent background biometric sensor data from the user's mobile computing device following the treatment initiation flag;
receiving a positive outcome flag via the clinician portal indicating a clinical improvement in the user;
executing a second retrospective analysis via the machine learning model on the subsequent background biometric sensor data captured between the treatment initiation flag and the positive outcome flag;
identifying a physiological stabilization pattern in the subsequent background biometric sensor data indicating treatment efficacy; and
applying the identified physiological stabilization pattern to actively treated patients to dynamically predict the efficacy of a prescribed treatment prior to an improvement in the foreground task completion score.

5. The method of claim 1, wherein generating the predictive trajectory vector further comprises:
calculating a specific temporal prognosis indicating a projected timeframe before the onset of severe neurological degradation based on the historical dataset.

6. The method of claim 1, wherein the machine learning model is further trained by:
receiving a normal health flag via the clinician portal indicating a baseline evaluation period of a predefined duration wherein a patient demonstrates stable health; and
modeling a baseline biometric signature of a normal brain based on the background biometric sensor data associated with the normal health flag to increase the machine learning model's accuracy in contrasting healthy users against neurologically compromised users.

7. The method of claim 1, wherein the sub-perceptual biometric anomaly comprises an increase in the millisecond-level inter-tap latency occurring simultaneously with a targeted subset of the spatial coordinate data indicating hemispatial interaction neglect.

8. The method of claim 1, wherein processing the background biometric sensor data further comprises:
combining the captured foreground interaction data with the background biometric sensor data to form a synergistic multidimensional dataset; and
wherein the trained machine learning model processes the synergistic multidimensional dataset to detect the sub-perceptual biometric anomaly and generate the predictive trajectory vector.

9. The method of claim 4, wherein executing the second retrospective analysis comprises:
initiating an algorithmic review of the subsequent background biometric sensor data starting chronologically at a date associated with the treatment initiation flag and advancing forward in time until the machine learning model identifies the physiological stabilization pattern, thereby determining a minimum time threshold required to identify treatment efficacy prior to the receipt of the positive outcome flag.

10. The method of claim 2, wherein executing the retrospective analysis on the historical dataset of the specific patient comprises:
initiating an algorithmic review of the historical dataset starting chronologically from an earliest recorded baseline session of the specific patient and advancing forward in time until the machine learning model recognizes a pattern constituting the early-warning biometric signature.

11. A system for predictive neurological monitoring and clinical decision support via mobile device sensor fusion, the system comprising:
a mobile computing device comprising a display screen, a capacitive touch digitizer, a multi-axis accelerometer, a gyroscope, a native operating system event logger, and a wireless transceiver;
a remote cloud-based server in communication with the mobile computing device; and
a secure clinician portal in communication with the remote cloud-based server;
wherein the mobile computing device is configured to render a digitized cognitive testing module on the display screen, capture foreground interaction data as a user interacts with the digitized cognitive testing module, simultaneously capture background biometric sensor data, and transmit the foreground interaction data and the background biometric sensor data to the remote cloud-based server;
wherein capturing the background biometric sensor data comprises polling continuous multi-axis motion data utilizing the accelerometer and the gyroscope to extract micro-oscillation and device sway metrics, logging spatial coordinate data and haptic pressure data utilizing the capacitive touch digitizer, and timestamping individual screen interactions utilizing the native operating system event logger to extract millisecond-level inter-tap latency and motor planning metrics;
wherein the remote cloud-based server comprises a machine learning architecture configured to calculate a foreground task completion score based on the foreground interaction data, and process the background biometric sensor data via a trained machine learning model to detect a sub-perceptual biometric anomaly;
wherein the machine learning model is trained on a historical dataset comprising background biometric sensor data from a plurality of patients correlated with supervised clinical diagnosis flags;
wherein the remote cloud-based server is further configured to generate a predictive trajectory vector indicating a future onset of a neurological condition based on the detected sub-perceptual biometric anomaly prior to a macroscopic decline in the user's foreground task completion score; and
wherein the secure clinician portal is configured to output an alert and a corresponding specific treatment plan recommendation based on the generated predictive trajectory vector.

12. The system of claim 11,
wherein the secure clinician portal is configured to receive a supervised clinical diagnosis flag establishing a date of formal neurological diagnosis for a specific patient; and
wherein the remote cloud-based server is configured to train the machine learning model by executing a retrospective analysis on the historical dataset of the specific patient, the retrospective analysis comprising initiating an algorithmic review of the historical dataset starting chronologically from an earliest recorded baseline session of the specific patient and advancing forward in time until the machine learning model recognizes a pattern constituting an early-warning biometric signature occurring prior to the date of formal neurological diagnosis.

13. The system of claim 11,
wherein the secure clinician portal is further configured to receive a treatment initiation flag indicating the specific treatment plan has been prescribed, and subsequently receive a positive outcome flag indicating a clinical improvement in the user; and
wherein the remote cloud-based server is configured to execute a second retrospective analysis on subsequent background biometric sensor data continuously received following the treatment initiation flag, the second retrospective analysis comprising initiating an algorithmic review starting chronologically at a date associated with the treatment initiation flag and advancing forward in time until the machine learning model identifies a physiological stabilization pattern indicating treatment efficacy prior to an improvement in the foreground task completion score.

14. The system of claim 11,
wherein the remote cloud-based server is configured to combine the captured foreground interaction data with the background biometric sensor data to form a synergistic multidimensional dataset; and
wherein the trained machine learning model processes the synergistic multidimensional dataset to detect the sub-perceptual biometric anomaly and generate the predictive trajectory vector.

15. The system of claim 11,
wherein the secure clinician portal is configured to receive a normal health flag indicating a baseline evaluation period of a predefined duration wherein a patient demonstrates stable health; and
wherein the machine learning architecture of the remote cloud-based server is configured to model a baseline biometric signature of a normal brain based on the background biometric sensor data associated with the normal health flag to increase the machine learning model's accuracy in contrasting healthy users against neurologically compromised users.

16. The system of claim 11, wherein the remote cloud-based server is configured to generate the predictive trajectory vector by calculating a specific temporal prognosis indicating a projected timeframe before the onset of severe neurological degradation based on the historical dataset.

17. The system of claim 11, wherein the mobile computing device is configured to continuously capture the background biometric sensor data during non-clinical, gamified application usage independently of the active rendering of the digitized cognitive testing module.

18. The system of claim 11, wherein the sub-perceptual biometric anomaly detected by the remote cloud-based server comprises an increase in the millisecond-level inter-tap latency occurring simultaneously with a targeted subset of the spatial coordinate data indicating hemispatial interaction neglect.
